SEQUENCE LISTING (2 sequences)

SEQ ID NO: 1 - DNA, 33 bp, Artificial Sequence
  Synthetic Construct
atataggcgcgccgaattcgccaccatggaatt

SEQ ID NO: 2 - DNA, 33 bp, Artificial Sequence
  Synthetic Construct
atatagtttaaacattacacggcgatctttccg

(19) United States
(12) Patent Application Publication
(10) Pub. No.: US 2023/0390277 A1
(43) Pub. Date: Dec. 7, 2023

(54) METHODS TO SENSITIZE TUMORS TO TREATMENT BY IMMUNOTHERAPY

(71) Applicant: Ohio State Innovation Foundation, Columbus, OH (US)

(72) Inventors: Nicholas DENKO, Dublin, OH (US); Ioanna PAPANDREOU, Columbus, OH (US); Zihai LI, Powell, OH (US); Martin BENEJ, Upper Arlington, OH (US); No Joon SONG, Upper Arlington, OH (US)

(21) Appl. No.: 18/033,178

(22) PCT Filed: Aug. 23, 2021
(86) PCT No.: PCT/US2021/047100

§ 371 Date: Apr. 21, 2023

Related U.S. Application Data
(60) Provisional application No. 63/094,621, filed on Oct. 21, 2020.

(51) Int. Cl. A61K 31/472; A61K 31/4725; A61K 31/517; A61P 35/00; A61K 45/06; A61K 39/395

(52) U.S. Cl. CPC A61K 31/472; A61K 31/4725; A61K 31/517; A61P 35/00; A61K 45/06; A61K 39/3955

(57) ABSTRACT

Disclosed herein are methods for treating cancer in a subject that comprise administering to the subject a therapeutically effective amount of one or more compounds disclosed herein, or pharmaceutical composition comprising one or more compounds disclosed herein to inhibit mitochondrial oxygen consumption in a cancer cell; and administering an immunotherapy to treat the cancer.

### CROSS-REFERENCE TO RELATED APPLICATIONS

The application claims the benefit of U.S. Provisional Application No. 63/094,621, filed Oct. 21, 2020, which is hereby incorporated herein by reference in its entirety.

### STATEMENT REGARDING FEDERALLY SPONSORED RESEARCH OR DEVELOPMENT

This disclosure was made with government support under Grants P01 CA067166, 1R01CA163581, and R01CA255334 awarded by National Institutes of Health. The government has certain rights in the invention.

### BACKGROUND

Hypoxia is a common microenvironmental feature of solid tumors (Brown, J. M. et al. Cancer Res 58, 1408-1416 (1998)) that exists because the supply of oxygen is insufficient to meet the metabolic demand of the tumor (Epstein, T. et al. Cancer Metab 2, 7 (2014) and Semenza, G. L. et al. The Journal of clinical investigation 123, 3664-3671 (2013)). The poorly formed tumor blood vessels make it difficult to therapeutically increase oxygen delivery to reduce hypoxia (Harrison, D. K. et al. Adv Exp Med Biol 812, 25-31 (2014)).

Even though tumor hypoxia has been studied preclinically, clinical approaches designed to overcome hypoxia have had disappointing results. The most widely studied strategy has been to increase delivery of oxygen to tumors. Strategies designed to deliver more oxygen to the tumor, deliver oxygen-mimetics, or deliver drugs with preferential toxicity towards hypoxic cells have all failed in human trials. These attempts are limited by the inherent nature of the poorly formed and poorly functioning tumor vascular tree. Vessels in transplanted and spontaneous tumors have blind ends, breaks, and tortuous paths, all of which reduce laminar flow and decrease oxygen delivery.

Because hypoxic tumors in humans have been shown to be difficult to treat, there is therefore a need for compounds and methods that can treat hypoxic tumors and increase patient survival. The compounds, compositions, and methods disclosed herein address these and other needs.

The compositions and methods disclosed herein address these and other needs.

### SUMMARY

Described herein are methods for treating cancer in a subject comprising: administering to the subject a therapeutically effective amount of one or more compounds disclosed herein; or pharmaceutical composition comprising one or more compounds disclosed herein to inhibit mitochondrial oxygen consumption in a cancer cell; and administering an immunotherapy to treat the cancer. The compounds described herein can be represented by a structure having the Formula I:

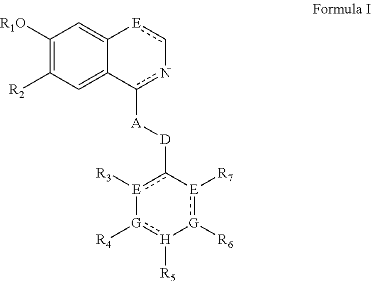

wherein A and D are independently present or absent and are independently selected from CR′R″, NR′, and O, wherein R′ and R″ and are independently for each occurrence selected from hydrogen, hydroxyl, halogen, amine, alkylamine, C₁-C₆ alkyl, C₁-C₆ alkyl halide, C₁-C₆ alkoxy, or R′ and R″ combine together with the atom to which they are attached form a carbonyl group; E, G, and H are independently selected from C, N, O, and S; R¹ and R² are independently selected from hydrogen, C₁-C₆ alkyl, C₁-C₆ alkyl halide, C₁-C₆ alkoxy, and C₁-C₆ alkylamine; R³ to R⁷ are independently selected from hydrogen, hydroxyl, C₁-C₆ alkyl, C₁-C₆ alkyl halide, C₁-C₆ alkoxy, and C₁-C₆ alkylamine or R³ and R⁴ or R⁴ and R⁵ or R⁵ and R⁶ or R⁶ and R⁷ combine together with the atoms to which they are attached form a C₁-C₆ aryl or heteroaryl, or C₅-C₈ cycloalkenyl or heterocycloalkenyl; wherein R³ to R⁷ are independently unsubstituted or substituted with hydroxyl, halogen, C₁-C₃ alkyl, C₁-C₃ alkenyl, or C₁-C₃ alkyl halide; and ———— represents a bond and is independently for each occurrence absent or present, wherein when A is CH₂ and D is absent, then R¹, R², R⁴, and R⁵ are not simultaneously OMe or when A is CH₂, D is absent, R¹ and R² are OMe, then R⁴ and R⁵ do not combine to form and unsubstituted aryl.

In some embodiments of Formula I, the compounds can be represented by a structu having the Formula as described herein, wherein F is selected from C and N.

In some embodiments of Formula I and I′, A is present and D is absent. In some embodiments, of Formula I and I′, both A and D are present, In some embodiments, of Formula I and both A and D are absent.

In some embodiments of Formula I and I′, can be selected from CR′R″ and O. In some embodiments of Formula I and I′, D can be selected from CR′R″ and O. In some embodiments of Formula I and I′, A and D can both be CR′R″. In some embodiments of Formula I and I′, A can be CR′R″ and D can be O.

In some embodiments of Formula I and F, R and R can be independently for each occurrence selected from hydrogen, hydroxyl, C₁-C₆ alkyl, or R′ and R″ combine together with the atom to which they are attached form a carbonyl. For example, R′ and R″ can be hydrogen. In some examples, R′ is hydrogen and at least one occurrence of R″ is hydroxyl. In other examples, at least one occurrence of R and R combine together with the atom to which they are attached form a carbonyl.

In some embodiments of Formula I and I′, R¹ can be selected from a C₁-C₆ alkyl. For example, R¹ can be selected from a C₁-C₂ alkyl.

In some embodiments of Formula I and I′, R² can be independently selected from hydrogen or a C₁-C₆ alkoxy. For example, R² can be selected from a C₁-C₂ alkoxy. In some examples, R² can be hydrogen.

In some embodiments of Formula I and I′, R³, R⁶, and R⁷ can be independently selected from hydrogen, hydroxyl, C₁-C₆ alkyl, C₁-C₆ alkyl halide, C₁-C₆ alkoxy, or R³ and R⁴ or R⁴ and R⁵ combine together with the atoms to which they are attached form a C₅-C₈ aryl or heteroaryl, or C₅-C₈ cycloalkenyl or heterocycloalkenyl, wherein R³ to R⁷ are independently unsubstituted or substituted with hydroxyl, halogen, C₁-C₃ alkyl, C₁-C₃ alkenyl, or C₁-C₃ alkyl halide. For example, R³ and R⁷ are hydrogen. In some examples, R⁴, R⁵, and R⁶ can be independently selected from hydrogen, hydroxyl, C₁-C₆ alkyl, C₁-C₆ alkyl halide, or C₁-C₆ alkoxy. In other examples, R³ and R⁴ or R⁴ and R⁵ combine together with the atoms to which they are attached form a C₆ aryl, a C₆ heteroaryl, or a C₅ heterocycloalkenyl.

In some embodiments, the cancer can be selected from colorectal cancer, breast cancer, bladder cancer, gastrointestinal cancer, brain cancer, cervical cancer, head and neck cancer, lung cancer, prostate cancer, skin cancer, esophageal cancer, thyroid cancer, adrenal gland cancer, bone cancer, testicular cancer, leukemia, myeloma, sarcoma, and lymphoma or melanoma.

In some embodiments, the immunotherapy can include an anti-cancer immunotherapy. In some embodiments, the immunotherapy can include one or more agents capable of immune checkpoint blockade. In some embodiments, the one or more agents capable of immune checkpoint blockade are immune checkpoint inhibitors, such as a CTLA-4 inhibitor, a PD-1 inhibitor, a PD-L1 inhibitor, new agents designed to block other immune checkpoints, or any combination thereof. In some embodiments, the one or more agents capable of immune checkpoint blockade are immune checkpoint inhibitors, such as a CTLA-4 inhibitor, a PD-1 inhibitor, a PD-L1 inhibitor, or any combination thereof.

In some embodiments, the one or more compounds, or the composition comprising the one or more compounds can be administered prior to, during, or after the immunotherapy is administered. In some embodiments, the one or more compounds, or the composition comprising the one or more compounds can be administered prior to, during, and after the immunotherapy is administered. In some embodiments, the one or more compounds, or the composition comprising the one or more compounds can be administered prior to and during the immunotherapy is administered. In some embodiments, the one or more compounds, or the composition comprising the one or more compounds can be administered prior to and after the immunotherapy is administered. In some embodiments, the one or more compounds, or the composition comprising the one or more compounds can be administered prior to the immunotherapy is administered. In some embodiments, the one or more compounds, or the composition comprising the one or more compounds can be administered during the immunotherapy is administered. In some embodiments, the one or more compounds, or the composition comprising the one or more compounds can be administered after the immunotherapy is administered.

In some embodiments, the one or more compounds, or the composition comprising the one or more compounds can be administered at least 16 hours before the immunotherapy is administered. In some embodiments, the method can further comprise administering a chemotherapeutic drug.

Pharmaceutical composition or formulation comprising a compound disclosed herein are also disclosed. In some examples, the pharmaceutical compositions comprises papaverine.

In some embodiments, the compound or composition can be administered by one or more routes selected from buccal, sublingual, intravenous, subcutaneous, intradermal, transdermal, intraperitoneal, oral, eye drops, parenteral, or topical administration.

The details of one or more embodiments of the disclosure are set forth in the accompanying drawings and the description below. Other features, objects, and advantages of the disclosure will be apparent from the description and drawings, and from the claims.

### DESCRIPTION OF DRAWINGS

FIG. 1 shows PPV derivatives' activity for inhibiting OCR at 10 μM and inhibiting PDE10A at 1 μM. The half molecule DMIQ is inactive for either assay, while PPV OCR IC₅₀ is 200× that of rotenone (rotenone activity shown at 0.1 μM).

FIG. 2A-2B shows the relative activities of PPV, and derivatives SMV23 and SMV32 for OCR (2A) and PDEIWA (2B) inhibition. NB for both activities new compounds have over a 10-fold separation.

FIG. 3 shows that PPV and SMV32 both enhance the effectiveness of suboptimal PD-1 blockade. MC₃₈ tumors grown in C₅₇B16 mice were treated as indicated with irrelevant antibody, anti PD-1, or anti PD-1 after PPV or SMV32 n=5-7, P=<0.05* for PPV and <0.01** for SMV32.

FIG. 4 shows that SMV32 is superior to PPV for sensitizing tumors to PD-1 blockade. C₅₇B16 mice bearing CMT167 flank lung tumors were treated as indicated on days 10 and 13 and relative volume followed. N=5 group, P<0.05 for PD1 vs PPV->PD1, P<0.01 for PD1 vs SMV32->PD1 by T test.

FIG. 5 shows that SMV32 is superior to PPV for sensitizing tumors to PD-1 blockade in resistant LLC tumors. LLC tumor bearing mice were treated as indicated on days 5, 8 and and relative growth followed. P=NS for PD-1 vs PPV->PD1, 0.04 for PD-i vs SMV32->PD-1.

FIG. 6 shows the impact of SMV32 on T-cell exhaustion. Panel A Tumor infiltrating immune cells isolated from Day 15 MC₃₈ tumors (n=4/g) were stained for lineage (CD45, CD3, CD8, CD5, and CD11b) and T cell exhaustion (PD1, Tim3, Slamf6, TOX, Tcf1, CD44, CD62L, CTLA4, Lag-3, Klrg1, T-bet, and Ki-67) (93-96). Cells were analyzed by Cytek Aurora multi-spectral FACS, and Umap generated based on concatenated file from each 4 FSC files without variation in cell number. Limp figure was generated by OMIQ with CD8 T cells (CD45+, CD3+, CD8+, CD11b−, CD4−). CD8 population was divided into 15 clusters based on T cell markers. Expression signatures marked (ovals) show progenitor exhausted T cells and terminally exhausted T cells. Panel B Statistical significance of clusters between control and SMV32 treated group were assessed by edgeR and SAM (n=4 mice per group).

FIG. 7A-7H shows pODD-Luc signal increases under hypoxia in vitro and in vivo. (7A) Representative in vitro luciferase assay showing reporter signal increase in response to 24 h hypoxia and loss of signal after 10 min reoxygenation in MP2 pODD cells; values were normalized against hypoxic treatment. P values were calculated against hypoxia by t test; (n=3 replicates). (7B) Western blot of luciferase reporter protein levels in HeLa pODD cells; (n=3). (7C) DMOG dose response analysis in MP2 pODD cell lysates after the indicated treatments; (n=3). (7D) Luciferase assay showing signal increase in response to 1mM DMOG in MP2 pODD and no increase in MP2 CMV cells. P values were calculated against control by t test; (n=3). (7E) Proteasome inhibition increases MP2 pODD signal after one hour of 10 tiM its MG132; (n=3). (7F) In vivo luciferase activity of individual HeLa pODD heterotopic flank tumors grown in nu/nu mice with values of photon flux:is normalized to tumor volume; (n=replicates). (7G) Representative in vivo imaging assay showing pODD-Luc signal in HeLa. pODD flank tumors; (n=4). (7H) Photon flux in MP2 pODD flank tumors 30 min after animals breathed either medical air or carbogen as indicated (n=4 tumors). Error bars are ±SEM. *P<0.05; **P<0.01; ***P<0.001.

FIG. 8A-8E shows pharmacological manipulation of ODD-luc activity in vivo. In vivo bioluminescent image quantification of pODD-Luc signal in heterotopic flank tumors grown from NIP2 ODD cells in immunocompromised mice, after treatment with: (8A) 4 mg of DMOG I.P., (n=4 tumors); (8B) 1 mg/kg of bortezomib I.P., (n=5); (8C) 2 g/kg of glucose intraperitoneally (I.P.) following overnight fasting, (n=5); (8D) 20 mg/kg of 2,4-DNP I.P., heterotopic tumors (n=6); (8E) 75 mg/kg of DCA I.P., (n=4). Error bars are ÷SEM. P values were calculated against baseline by paired t test. *P<0.05; **P<0.01.

FIG. 9A-9F shows mitochondrial inhibitors PPV and SMV-32 shows reduced ODDluc activity (hypoxia) in vivo. (9A) Acute in vivo imaging pODD-Luc quantification in nu./nu mice bearing orthotopic MP2 pODD or CMV tumors in response to a intraperitoneal treatment with D-luciferin (n=3); (9B) in vivo Luciferase activity of MP2 CMV in orthotopic tumors treated with 2 mg/kg PPV or vehicle by tail vein injection (n=4); (9C) In vivo luciferase activity of MP2 pODD in orthotopic tumors treated with PPV or vehicle by tail vein injection (n=4), P values were calculated against vehicle; (9D) In vivo luciferase activity of MP2 pODD in heterotopic flank tumors treated with PPV by tail vein injection (n=5); (9E) In vivo luciferase activity of Hela pODD-Luc in heterotopic flank tumors treated with PPV by tail vein injection (n=3); (9F) In vivo luciferase activity of iMP2 pODD in orthotopic tumors treated with SMV-32 or vehicle by tail vein injection (n=4), P values were calculated against vehicle. Error bars are ±SEM. Unless otherwise indicated, P values were calculated against baseline by paired t test. *P<0.05; **P<0.01; ***P<0.001.

FIG. 10A-10D shows mitochondrial inhibitor PPV increases oxygenation in spontaneous canine soft tissue sarcoma. (10A) Scheme for imaging patients to determine effect of PPV on tumor oxygenation. (10B) Representative overlay image from hip sarcoma in patient 7 with pseudocolor indicating changes of 2 ms (yellow), 5 ins (orange) or 10 ms (red). (10C) Histogram showing baseline (blue) and post-PPV (red) T2* values for patient 7. (10D) Waterfall summary of first 9 patients imaged showing fractional tumor volumes displaying an increase of either 2, 5 or 10 ms in T2*. Dose of PPV indicated for each animal.

FIG. 11A-11F shows representative western blot of luciferase reporter protein level (11A) and qualitative bioluminescence image following addition of 150 μg/ml D-luciferin (11B) in MP2 pODD cells exposed to 24 hours of 1% O2 with and without 10-minute reoxygenation. (11C-11F) show in vivo IVIS imaging quantification of pODD-Luc signal in immunocompromised mice bearing MP2 pODD (11C and 11D) or HeLa pODD (11E and 11F) flank tumors, treated with: (11C) vehicle by tail vein, MP2 pODD tumors, n=4; (11D) 2 mg/kg PPV intraperitoneally, MP2 pODD tumors, n=4; (11E) 4 mg/kg PPV by tail vein, HeLa pODD tumors, n=3; and (11F) 2 mg/kg SMV-32 by tail vein, HeLa pODD, n=3. Error bars are ±SEM. *p<0.05.

FIG. 12 shows images of uncropped western blot.

Like reference symbols in the various drawings indicate like elements.

### DETAILED DESCRIPTION

A number of embodiments of the disclosure have been described. Nevertheless, it will be understood that various modifications may be made without departing from the spirit and scope of the invention. Accordingly, other embodiments are within the scope of the following claims.

Definitions

To facilitate understanding of the disclosure set forth herein, a number of terms are defined below. Unless defined otherwise, all technical and scientific terms used herein generally have the same meaning as commonly understood by one of ordinary skill in the art to which this disclosure belongs. Publications cited herein and the materials for which they are cited are specifically incorporated by reference.

Throughout the description and claims of this specification the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a compound” includes mixtures of two or more such compounds, reference to “an additional chemotherapy agent” includes mixtures of two or more such agents, reference to “the composition” includes mixtures of two or more of such compositions, and the like.

Ranges can be expressed herein as from “about” one particular value, and/or to “about” another particular value. When such a range is expressed, another aspect includes from the one tri particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed.

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human.

By “reduce” or other forms of the word, such as “reducing” or “reduction,” is meant lowering of an event or characteristic. It is understood that this is typically in relation to some standard or expected value, in other words it is relative, but that it is not always necessary for the standard or relative value to be referred ta For example, “reduce oxygen consumption in the tumor cells” can refer to a decrease in the amount of oxygen consumed relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The terms “treatment,” “treat,” “treating,” and grammatical variations thereof, are used interchangeably herein to refer to the medical management of a patient with the intent to cure, ameliorate; stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

An “effective amount” of a compound or composition disclosed herein is that amount which is necessary to carry out the compound's or composition's function of ameliorating, diminishing, reversing, treating or preventing a condition, disease or disorder. Such amelioration only requires a reduction or alteration, not necessarily elimination. As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient. “Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

As used herein, the term “pharmaceutically acceptable” component can refer to a component that is not biologically or otherwise undesirable, i.e., the component may be incorporated into a pharmaceutical formulation of the invention and administered to a subject as described herein without causing any significant undesirable biological effects or interacting in a deleterious manner with any of the other components of the formulation in which it is contained. When the term “pharmaceutically acceptable” is used to refer to an excipient, it is generally implied that the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as desciibed further herein.

As used herein, “pharmaceutically acceptable salt” is a derivative of the disclosed compound in which the parent compound is modified by making inorganic and organic, non-toxic, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are typical, where practicable. Salts of the present compounds further include solvates of the compounds and of the compound salts.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesuifonic, ethane disulfonic, oxalic, isethionic, HOOC—(CH2)n—COOH where n is 0-4, and the like, or using a different acid that produces the same countetion. Lists of additional suitable salts may be found, e.g., in Remington's Pharmaceutical Sciences, 17th ed., Mack Publishing Company, Easton, Pa., p. 1418 (1985).

Also, as used herein, the term “pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

A “control” is an alternative subject or sample used in an experiment for comparison purposes. A control can be “positive” or “negative.”

Chemical Definitions

The term “alkyl,” as used herein, refers to saturated straight, branched, cyclic, primary, secondary or tertiary hydrocarbons, including those having 1 to 20 atoms. In some embodiments, alkyl groups will include C₁-C₁₂, C₁-C₁₀, C₁-C₆, C₁-C₃, C₂, or C₁ alkyl groups. Examples of C₁-C₁₀ alkyl groups include, but are not limited to, methyl, ethyl, propyl, 1-methylethyl, butyl, 1-methylpropyl, 2-methylpropyl, 1,1-dimethylethyl, pentyl, -methylbutyl, 2-methylbutyl, 3-methylbutyl, 2,2-dimethylpropyl, -ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, -ethyl-2-methylpropyl, heptyl, octyl, 2-ethylhexyl, nonyl and decyl groups, as well as their isomers. Examples of C₁-C₄-alkyl groups include, for example, methyl, ethyl, propyl, 1-methylethyl, butyl, 1-methylpropyl, 2-methylpropyl and 1,1-dimethylethyl groups.

Cyclic alkyl groups or “cycloalkyl” groups, which are encompassed alkyl, include cycloalkyl groups having from 3 to 10 carbon atoms. Cycloalkyl groups can include a single ring, or multiple condensed rings. In some embodiments, cycloalkyl groups include C₃-C₄, C₄-C₇, C₅-C₇, C₄-C₆, or C₅-C₆ cyclic alkyl groups. Non-limiting examples of cycloalkyl groups include adamantyl, cyclopropyl, cyclobutyl, cyclopentyl, cyclohexyl, cycloheptyl, cyclooctyl and the like,

Alkyl groups can be unsubstituted or substituted with one or more moieties selected from the group consisting of alkyl, halo, haloalkyl, hydroxyl, carboxyl, acyl, a.cyloxy, amino, alkyl- or dialkylamino, amido, arylamino, alkoxy, aryloxy, nitro, cyano, azido, thiol, imino, sulfonic acid, sulfate, sulfonyl, sulfanyl, sulfinyl, sulfamonyl, ester, phosphonyl, phosphinyl, phosphoryl, phosphine, thioester, thioether, acid halide, anhydride, oxime, hydrazine, carbamate, phosphoric acid, phosphate, phosphonate, or any other viable functional group that does not inhibit the biological activity of the compounds of the invention, either unprotected, or protected as necessary, as known to those skilled in the art, for example, as described in Greene, et al., Protective Groups in Organic Synthesis, John Wiley and Sons, Third Edition, 1999, hereby incorporated by reference.

Terms including the term “alkyl,” such as “alkylcycloalkyl,” “cycloalkylalkyl,” “alkylamino,” or “dialkylamino,” will be understood to comprise an alkyl group as defined is above linked to another functional group, where the group is linked to the compound through the last group listed, as understood by those of skill in the art.

The term “alkenyl,” as used herein, refers to both straight and branched carbon chains which have at least one carbon-carbon double bond. In some embodiments, alkenyl groups can include C₂-C₂₀ alkenyl groups, In other embodiments, alkenyl can include C₂-C₁₂, C₂-C₈, C₂-C₆ or C₂-C₄ alkenyl groups. In one embodiment of alkenyl, the number of double bonds is 1-3, in another embodiment of alkenyl, the number of double bonds is one or two. Other ranges of carbon-carbon double bonds and carbon numbers are also contemplated depending on the location of the alkenyl moiety on the molecule. “C₂-C₁₀-alkenyl” groups may include more than one double bond in the chain. The one or more unsaturations within the alkenyl group may be located at any position(s) within the carbon chain as valence permits. In some embodiments, when the alkenyl group is covalently bound to one or more additional moieties, the carbon atom(s) in the alkenyl group that are covalently bound to the one or more additional moieties are not part of a carbon-carbon double bond within the alkenyl group. Examples of alkenyl groups include, but are not limited to, ethenyl, 1-propenyl, 2-propenyl, 1-methyl-ethenyl, 1-butenyl, 2-butenyl, 3-butenyl, 1-methyl-1-propenyl, 2-methyl-1-propenyl, 1-methyl-2-propenyl, 2-methyl-2-propenyl; 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1.-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl.-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyll, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyll, 1,1,2-trimethyl-2-propenyl, 1-ethyl-1-methyl-2-propenyl, 1-ethyl-2-methyl-1-propenyl and 1-ethyl-2-methyl-2-propenyl groups.

The term “alkynyl,” as used herein, refers to both straight and branched carbon chains which have at least one carbon-carbon triple bond. In one embodiment of alkynyl, the number of triple bonds is 1-3; in another embodiment of alkynyl, the number of triple bonds is one or two. In some embodiments, alkynyl groups include from C₂-C₂₀ alkynyl groups. In other embodiments, alkynyl groups may include C₂-C₁₂, C₂-C₁₀, C₂-C₈, C₂-C₆ or C₂-C₄ alkynyl groups. Other ranges of carbon-carbon triple bonds and carbon numbers are also contemplated depending on the location of the alkenyl moiety on the molecule. For example, the term “C₂-C₁₀-alkenyl” as used herein refers to a straight-chain or branched unsaturated hydrocarbon group having 2 to 10 carbon atoms and containing at least one triple bond, such as ethynyl, prop-1-yn-1-yl, prop-2-yn-1-yl, n-but-1-yn-1-yl, n-but-1-yn-3-yl, n-but-1-yn-4-yl, n-but-2-yn-1-yl, n-pent-i-yn-1-yl, n-pent-1-yn-4-yl, n-pent-1-yn-5-yl, n-pent-2-yn-1-yl, n-pent-2-yn-4-yl, n-pent-2-yn-5-yl, 3-methyllbut-1-yn-3-yl, 3-methylbut-1-yn-4-yl, n-hex-1-yn-1-yl, n-hex-1-yn-3-yl, n-hex-2-yn-4-yl, n-hex-2-yn-5-yl, n-hex-2-yn-6-yl, n-hex-3-yn-1-yl, n-hex-3-yn-2-yl, 3-methylpent-1-yn-1-yl, 3-methylpent-1-yn-3-yl, 3-methylpent-1-yn-4-yl, 3-methylpent-1-yn-5-yl, 4-methylpent-1-yn-1-yl, 4-methylpent-2-yn-4-yl, and 4-methylpent-2-yn-5-yl groups.

The term “haloalkyl” or “alkylhalide,” as used herein refers to an alkyl group, as defined above, which is substituted by one or more halogen atoms. In some instances, the haloalkyl group can be an alkyl group substituted by one or more fluorine atoms. In certain instances, the haloalkyl group can be a perfluorinated alkyl group. For example C₁-C₄-haloalkyl includes, but is not limited to, chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, and pentafluoroethyl. The term “alkoxy,” as used herein, refers to alkyl-O—, wherein alkyl refers to an alkyl group, as defined above. Similarly, the terms “alkenyloxy,” “alkynyloxy,” “haloalkoxy,” “haloalkenyloxy,” “haloalkynyloxy,” “cycloalkoxy,” “cycloalkenyloxy,” “halocycloalkoxy,” and “halocycloalkenyloxy” refer to the groups alkenyl-O—, alkynyl-O—, haloalkyl-O—, haloalkenyl-O—, haloalkynyl-O—, cycloalkyl-O—, cycloalkenyl-O—, halocycloalkyl-O—, and halocycloalkenyl-O—, respectively, wherein alkenyl, alkynyl, haloalkyl, haloalkenyl, haloalkynyl, cycloalkyl, cycloalkenyl, halocycloalkyl, and halocycloalkenyl are as defined above. Examples of C₁-C₆-alkoxy include, but are not limited to, methoxy, ethoxy, C₂H₅-CH₂O—, (CH₃)2CHO—, n-butoxy, C₂H₅—CH(CH₃)O—, (CH₃)₂CH—CH₂O—,(CH₃)₃CO—, n-pentoxy, 1-methylbutoxy, 2-methylbutoxy, 3-methylbutoxy, 1,1-dimethylpropoxy, 1,2-dimethylpropoxy, 2,2-dimethyl-propoxy, 1-ethylpropoxy, n-hexoxy. 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy, and 1-ethyl-2-methylpropoxy. The terms “alkylamino” and “dialkylamino,” as used herein, refer to alkyl-NH— and (alkyl)2N— groups, where alkyl is as defined above. Similarly, the terms “haloalkylamino” and “halodialkylamino” refer to haloalkyl-NH— and (haloalkyl)₂—NH—, where haloalkyl is as defined above.

The term “aryl,” as used herein, refers to a monovalent aromatic carbocyclic group of from 6 to 14 carbon atoms. Aryl groups can include a single ring or multiple condensed rings. In some embodiments, aryl groups include C₆-Cio aryl groups. Aryl groups include, but are not limited to, phenyl, biphenyl, naphthyl, tetrahydronaphtyl, phenylcyclopropyl and indanyl. Aryl groups may be unsubstituted or substituted by one or more moieties selected from halogen, cyano, nitro, hydroxy, mercapto, amino, alkyl, alkenyl, alkynyl, cycloalkyl, cycloalkenyl, haloalkyl, haloalkenyl, haloalkynyl, halocycloalkyl, halocycloalkenyl, alkoxy, alkenyloxy, alkynyloxy, haloalkoxy, haloalkenyloxy, haloalkynyloxy, cycloalkoxy, cycloalkenyloxy, halocycloalkoxy, halocycloalkenyloxy, alkylthio, haloalkylthio, cycloalkylthio, halocycloalkylthio, allylsulfinyl, alkenylsulfinyl, alkynyl-sulfinyl, haloalkylsulfinyl, haloalkenylsulfinyl, haloalkynylsulfinyl, alkylsulfonyl, alkenylsulfonyl, alkynylsulfonyl, haloalkyl-sulfonyl, haloalkenykulfonyl, haloalkynylsulfonyl, alkylamino, alkenylamino, alkynylamino, di(alkyl)amino, di(alkenyl)-amino, di(alkynyl)amino, or trialkylsilyl.

The term “alkylaryl,” as used herein, refers to an aryl group that is bonded to a parent compound through a diradical alkylene bridge, (—CH₂—)ₙ, where n is 1-12 and where “aryl” is as defined above.

The term “alkylcycloalkyl,” as used herein, refers to a cycloalkyl group that is bonded to a parent compound through a diradical alkylene bridge, (—CH₂—)ₙ, where n is 1-12 and where “cycloalkyl” is as defined above. The term “cycloalkylalkyl,” as used herein, refers to a tri cycloalkyl group, as defined above, which is substituted by an alkyl group, as defined above.

The term “heteroaryl,” as used herein, refers to a monovalent aromatic group of from 1 to 15 carbon atoms (e.g., from 1 to 10 carbon atoms, from 2 to 8 carbon atoms, from 3 to 6 carbon atoms, or from 4 to 6 carbon atoms) having one or more heteroatoms within the ring. The heteroaryl group can include from 1 to 4 heteroatoms, from 1 to 3 heteroatoms, or from 1 to 2 heteroatoms. In some cases, the heteroatom(s) incorporated into the ring are oxygen, nitrogen, sulfur, or combinations thereof. When present, the nitrogen and sulfur heteroatoms may optionally be oxidized. Heteroaryl groups can have a single ring (e.g., pyridyl or furyl) or multiple condensed rings provided that the point of attachment is through a heteroaryl ring atom. Preferred heteroatyls include pyridyl, pyridazinyl, pyrimidinyl, pyrazinyl, triazinyl, pyrrolyil, quinazolinyl, quinoxalinyl, furanyl, thiophenyl, furyl, pyrrolyl, imidazolyl, oxazolyl, isoxazolyl, isothiazolyl, pyrazolyl, benzofuranyl, and (enzothiophenyl. Heteroaryl rings may be unsubstituted or substituted by one or more moieties as described for aryl above.

The term “alkylheteroaul,” as used herein, refers to a heteroaryl group that is bonded to a parent compound through a diradical alkylene bridge, (—CH₂—)ₙ, where n is 1-12 and where “heteroaryl” is as defined above.

The terms “cycloheteroalkyl,” “heterocyclyl,” “heterocyclic,” and “heterocycle” are used herein interchangeably, and refer to fully saturated or unsaturated, cyclic groups, for example, 3 to 7 membered monocyclic or 4 to 7 membered monocyclic; 7 to 11 membered bicyclic, or 10 to 15 membered tricyclic ring systems, having one or more heteroatoms within the ring. The heterocyclyl group can include from 1 to 4 heteroatoms, from 1 to 3 heteroatoms, or from 1 to 2 heteroatoms. In some cases, the heteroatom(s) incorporated into the ring are oxygen, nitrogen, sulfur, or combinations thereof. When present, the nitrogen and sulfur heteroatoms may optionally be oxidized, and the nitrogen heteroatoms may optionally be quaternized. The heterocyclyl group may be attached at any heteroatom or carbon atom of the ring or ring system and may be unsubstituted or substituted by one or more moieties as described for aryl groups above.

Exemplary monocyclic heterocyclic groups include, but are not limited to, pyrrolidinyi, pyrrolyl, pyrazolyl, oxetanyl, pyrazolinyl, imidazolyl, imidazolinyl, imidazolidinyl, oxazolyl, oxazolidinyl, isoxazolinyl, isoxazolyt, thiazolyt, thiadiazolyl, thiazolidinyl, isothiazolyl, isothiazolidirtyl, furyl, tetrahydrofuryl, thienyl, oxadiazolyl, piperidinyl, piperazinyl, 2-oxopiperazinyl, 2-oxopiperidinyl, 2-oxopyrrolodinyl, 2-oxoazepinyl, azepinyl, 4-piperidonyl, ire pyridinyl, pyrazinyl, pyridazinyl, tetrahydropyranyl, morpholinyl, thiamorpholinyl, thiamorpholinyl sulfoxide, thiamorpholinyl sulfone, 1,3-dioxotane and tetrahydro-1,1-dioxothienyl, triazolyl, triazinyl, and the like.

The term “alkylheterocyclyl” and “alkylcycloheteroalkyl” are used herein interchangeably, and refer to a heterocyclyl group that is bonded to a parent compound through a diradical alkylene bridge, (—CH₂—)ₙ, where n is 1-12 and where “heterocyclyl” is as defined above. The term “heterocyclylalkyl,” as used herein, refers to a heterocyclyl group, as defined above, which is substituted by an alkyl group, as defined above.

The term “halogen,” as used herein, refers to the atoms fluorine, chlorine, bromine and iodine. The prefix halo- (e.g., as illustrated by the term haloalkyl) refers to all degrees of halogen substitution, from a single substitution to a perhalo substitution (e.g., as illustrated with methyl as chloromethyl (—CH₂Cl), dichloromethyl (—CHCl₂), trichloromethyl (—CCl₃)).

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination etc.

Reference will now be made in detail to specific aspects of the disclosed materials, compounds, compositions, articles, and methods, examples of which are illustrated in the accompanying Examples and Figures.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the tri compositions and methods and in the steps or in the sequence of steps of the methods described herein without departing from the concept, spirit and scope of the disclosure. More specifically, it will be apparent that certain agents which are both chemically related may be substituted for the agents described herein while the same or similar results would be achieved. All such similar substitutes and modifications apparent to those skilled in the art are deemed to be within the spirit, scope and concept of the disclosure as defined by the appended claims,

Compounds

Disclosed herein are compounds and compositions for inhibiting mitochondrial oxygen consumption in a cancerous tissue. In certain embodiments, the compounds and compositions can treat hypoxic tumors in a subject. The compounds preferably comprise a planar portion as well as a flexible linkage.

In some aspects of the present disclosure, compounds represented by a structure having the Formula I are disclosed:

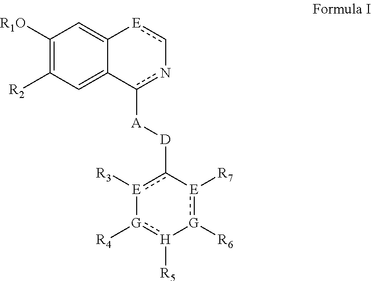

wherein
A and D can be independently present or absent and are independently selected from CR′R″, NR′, and O, wherein R′ and R″ are independently for each occurrence selected from hydrogen, hydroxyl, halogen, amine, alkylamine, C₁-C₆ alkyl; C₁-C₆ alkyl halide; C₁-C₆ alkoxy, or R′ and R″ combine together with the atom to which they are attached form a carbonyl group;
E, G, and H can be independently selected from C, N′, O, and S;
R¹ and R² can be independently selected from hydrogen, C₁-C₆ alkyl, C₁-C₆ alkyl halide, C₁-C₆ alkoxy, and C l-C₆ alkylamine;
R³ to R⁷ can be independently selected from hydrogen, hydroxyl, C₁-C₆ alkyl, C₁-C₆ alkyl halide, C₁-C₆alkoxy, and C₁-C₆ alkylamine or R³ and R⁴ or R⁴ and R⁵ or R⁵ and R⁶ or R⁶ and R⁷ combine together with the atoms to which they are attached form a C₅-C₈ aryl or heteroaryl, or C₅-C₈ cycloalkenyl or heterocycloalkenyl; wherein R³ to R⁷ are independently unsubstituted or substituted with hydroxyl, halogen, C₁-C₃ alkyl, C₁-C₃ alkenyl, or C₁-C₃ alkyl halide; and [symbol]
   represents a bond and is independently for each occurrence absent or present.

In certain embodiments of Formula I, when A is CH₂ and D is absent, then R¹, R², R⁴, and R⁵ are not simultaneously OMe. In certain other embodiments of Formula I, when A is CH₂, is absent, R¹ and R² are OMe, then R⁴ and R⁵ do not combine to form and unsubstituted aryl. In further embodiments of Formula I, R⁴ and R⁵ or R⁵ and R⁶ are not simultaneously OMe. In still further embodiments of Formula I, when A is CH₂ and D is absent, then R¹ and R² are not simultaneously OMe. For example, in some embodiments of Formula the compound is not papaverine or a

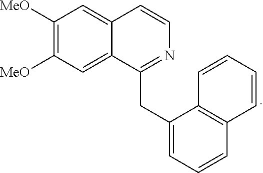 [SMILES: COc1cc2ccnc(Cc3cccc4ccccc34)c2cc1OC]

In some embodiments of Formula I, the compound can be represented by a structure having the Formula I′:

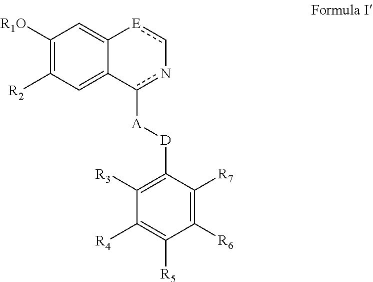

wherein A, D, R, R, and R¹ to R⁷ are as defined in Formula I.

For example, in some embodiments of Formula I′:
A and D are independently present or absent and are independently selected from CR′R″, NR″, and O, wherein R′ and R″ are independently for each occurrence selected from hydrogen, hydroxyl, halogen, amine, alkylamine, C₁-C₆ alkyl, C₁-C₆ alkyl halide, C₁-C₆ alkoxy, or Rand R combine together with the atom to which they are attached form a carbonyl group;
R¹ and R² are independently selected from hydrogen, C₁-C₆ alkyl, C₁-C₆ alkyl halide, C₁-C₆ alkoxy, and C t-C₆ alkylamine;
R³ to R⁷ are independently selected from hydrogen, hydroxyl, C₁-C₆ alkyl, C₁-C₆ alkyl halide, C₁-C₆ alkoxy, and C₁-C₆ alkylamine or R³ and R⁴ or R⁴ and R⁵ or R⁵ and R⁶ or R⁶ and R⁷ combine together with the atoms to which they are attached form a C₅-C₈ aryl or heteroaryl, or C₅-C₈ cycloalkenyl or heterocycloalkenyl; wherein R³ to R⁷ are independently unsubstituted or substituted with hydroxyl, halogen, C₁-C₃ alkyl, C₁-C₃ alkenyl, or C₁-C₃ alkyl halide; and [symbol]
   represents a bond and is independently for each occurrence absent or present,
wherein R⁴ and R⁵ or R⁵ and R6 are not simultaneously OMe, or when A is CH₂ and D is absent, then R¹ and R² are not simultaneously OMe.

In certain embodiments of Formula I, the compound can be represented by a structure having the Formula I-A to I-C:

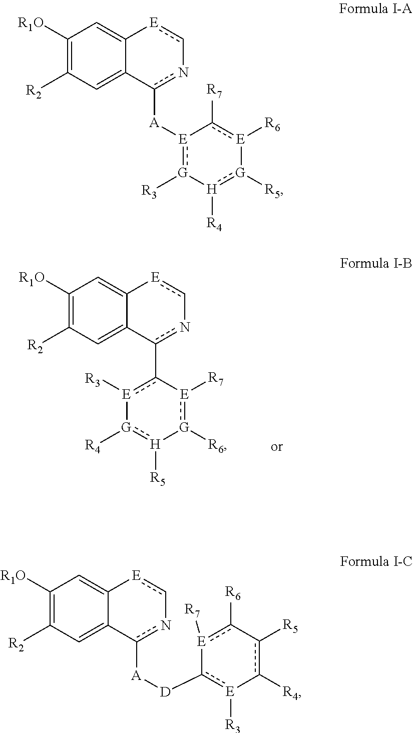

wherein
A is present in Formula I-A,
A and D are present in Formula I-C. and
A, D, E, G, H, R, R, and R i to R′ are as defined in Formula I.

For example, in some embodiments of Formula I-A to I-C:
A and D can be independently present or absent and are independently selected from CR′R″, NR′, and O, wherein R and R are independently for each occurrence selected from hydrogen, hydroxyl, halogen, amine, alkylamine, C₁-C₆ alkyl, C₁-C₆ alkyl halide, C₁-C₆ alkoxy, or R and R″ combine together with the atom to which they are attached form a carbonyl group;
E, G, and H can be independently selected from C, O, and S;
R¹ and R² can be independently selected from hydrogen, C₁-C₆ alkyl, C₁-C₆ alkyl halide, C₁-C₆ alkoxy, and C₁-C₆ alkylamine;
R³ to R⁷ can be independently selected from hydrogen, hydroxyl, C₁-C₆ alkyl, C₁-C₆ alkyl halide, C₁-C₆ alkoxy, and C₁-C₆ alkylamine or R³ and R⁴ or R⁴ and R⁵ or R⁵ and R⁶ or R⁶ and R⁷ combine together with the atoms to which they are attached form a C₅-C₈ aryl or heteroaryl, or C₅-C₈ cycloalkenyl or heterocycloalkenyl; wherein R³ to R⁷ are independently unsubstituted or substituted with hydroxyl, halogen, C₁-C₃ alkyl, C₁-C₃ alkenyl, or C₁-C₃ alkyl halide; and [symbol] represents a bond and is independently for each occurrence absent or present.

In certain embodiments of Formula I, the compound can be represented by a structure having the Formula I′-A to I′-C:

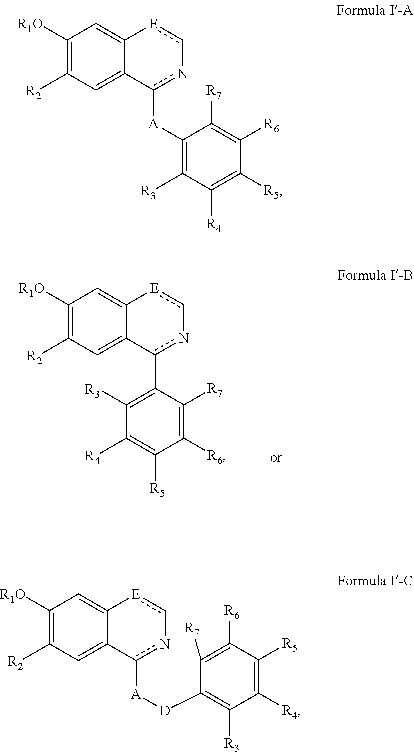

wherein
A is present in Formula I′-A,
A and D are present in Formula I′-C, and
A, D, R′, R″, and R¹ to R⁷ are as defined in Formula I.

For example, in some embodiments of Formula I′-A to I′-C:
A and D are independently present or absent and are independently selected from CR′R″, NR′, and O, wherein R′ and R″ are independently for each occurrence selected from hydrogen, hydroxyl, halogen, amine, alkylamine, C₁-C₆ alkyl, C₁-C₆ alkyl halide, C₁-C₆ alkoxy, or R′ and R″ combine together with the atom to which they are attached form a carbonyl group;
R¹ and R² are independently selected from hydrogen, C₁-C₆ alkyl, C₁-C₆ alkyl halide, C₁-C₆ alkoxy, and C₁-C₆ alkyl amine;
R³ to R⁷ are independently selected from hydrogen, hydroxyl, C₁-C₆ alkyl, C₁-C₆ alkyl halide, C₁-C₆ alkoxy, and C₁-C₆ alkylamine or R³ and R⁴ or R⁴ and R⁵ or R⁵ and R⁶ or R⁶ and R⁷ combine together with the atoms to which they are attached form a C₅-C₈ aryl or heteroaryl, or C₅-C₈ cycloalkenyl or heterocycloalkenyl; wherein R³ to R′ are independently unsubstituted or substituted with hydroxyl, halogen, C₁-C₃ alkyl, C₁-C₃ alkenyl, or C₁-C₃ alkyl halide; and [symbol]
   represents a bond and is independently for each occurrence absent or present,
wherein R⁴ and R⁵ or R⁵ and R⁶ are not simultaneously OMe, or when A is CH₂ and D is absent, then R¹ and R² are not simultaneously OMe.

In certain embodiments of Formula the compound can be represented by a structure having the Formula I-A-1:

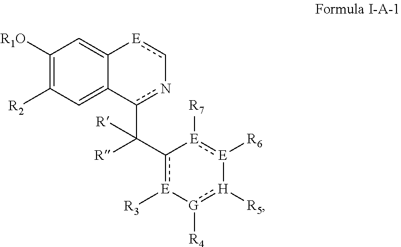

wherein
R′ and R″ are independently selected from hydrogen, hydroxyl, halogen, amine, alkylamine, C₁-C₆ alkyl, C₁-C₆ alkyl halide, C₁-C₆ alkoxy, or R′ and R″ combine together with the atom to which they are attached form a carbonyl;
R¹ is selected from hydrogen and C₁-C₆ alkyl;
R² is selected from hydrogen, hydroxyl, C₁-C₆ alkyl, and C₁-C₆ alkoxy; and
R³ to R⁷ are independently selected from hydrogen, hydroxyl, C₁-C₆ alkyl, Cr-Co alkyl halide, C₁-C₆ alkoxy, and C₁-C₆ alkylamine or R³ and R⁴ or R⁴ and R⁵ or R⁵ and R ° or R⁶ and R⁷ combine together with the atoms to which they are attached form a C₅-C₈ aryl or heteroaryl, or C₅-C₈ cycloalkenyl or heterocycloalkenyl,
wherein R³ to R⁷ are independently unsubstituted or substituted with hydroxyl, halogen, C₁-C₃ alkyl,Cr-C₃ alkenyl, or C 3 alkyl halide.

In certain embodiments of Formula 1, the compound can be represented by a structure having the Formula I′A-1:

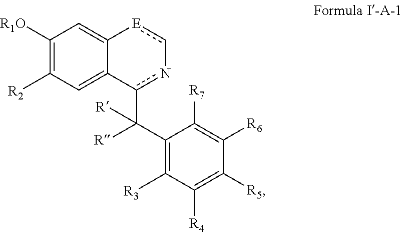

wherein
R′ and R″ are independently selected from hydrogen, hydroxyl, halogen, amine, alkylamine, C₆ alkyl, C₁-C₆ alkyl halide, C₁-C₆ alkoxy, or R′ and R″ combine together with the atom to which they are attached form a carbonyl;
R¹ is selected from hydrogen and C₁-C₆ alkyl;
R² is selected from hydrogen, hydroxyl, C₁-C₆ alkyl, and C₁-C₆ alkoxy; and
R³ to R⁷ are independently selected from hydrogen, hydroxyl, C₁-C₆ alkyl, C₁-C₆ alkyl halide, C₁-C₆ alkoxy, and C₁-C₆ alkylamine or R³ and R⁴ or R⁴ and R⁵ or R⁵ and R ° or R⁶ and R⁷ combine together with the atoms to which they are attached form a C₅-C₈ aryl or heteroaryl, or C₅-C₈ cycloalkenyl or heterocycloalkenyl,
wherein R³ to R⁷ are independently unsubstituted or substituted with hydroxyl, halogen, C₁-C₃ alkyl, C₁-C₃ alkenyl, or C₁-C₃ alkyl halide.

In certain embodiments of Formula I, the compound can be represented by a structure having the Formula I-B-1:

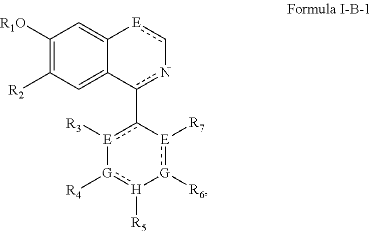

wherein
R¹ is selected from hydrogen and C₁-C₆ alkyl;
R² is selected from hydrogen, hydroxyl, C₁-C₆ alkyl, and C₁-C₆ alkoxy; and
R³ to R⁷ are independently selected from hydrogen, hydroxyl, C₁-C₆ alkyl, C₁-C₆ alkyl halide, C₁-C₆ alkoxy, and C₁-C₆alkylamine or R³ and R⁴ or R⁴ and R⁵ or R⁵ and R⁶ or R⁶ and R⁷ combine together with the atoms to which they are attached form a C₅-C₈ aryl or heteroaryl, or C₅-C₈ cycloalkenyl or heterocycloalkenyl,
wherein R³ to R⁷ are independently unsubstituted or substituted with hydroxyl, halogen, C₁-C₃ alkyl, C₃-C₃ alkenyl, or C₁-C₃ alkyl halide.

In certain embodiments of Formula I, the compound can be represented by a structure having the Formula I′-B-1:

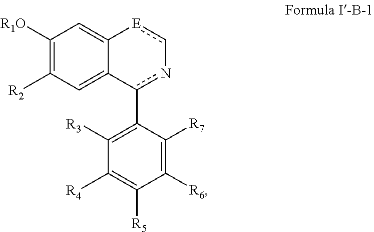

wherein
R⁴ is selected from hydrogen and C₁-C₆ alkyl;
R² is selected from hydrogen, hydroxyl, C₁-C₆ alkyl, and C₁-C₆ alkoxy; and
R³ to R⁷ are independently selected from hydrogen, hydroxyl, C₁-C₆ alkyl, C₁-C₆ alkyl halide, C₁-C₆alkoxy, and C₁-C₅ alkylamine or R³ and R⁴ or R⁴ and R⁵ or R⁵ and R⁶ or R⁶ and R⁷ combine together with the atoms to which they are attached form a C₅-C₈ aryl or heteroaryl, or C₅-C₈ cycloalkenyl or heterocycloalkenyl,
wherein R³ to R⁷ are independently unsubstituted or substituted with hydroxyl, halogen, C₁-C₃ alkyl, C₁-C₃ alkenyl, or C₁-C₃ alkyl halide.

In certain embodiments of Formula I, the compound can be represented by a structure icy having the Formula I-C-1:

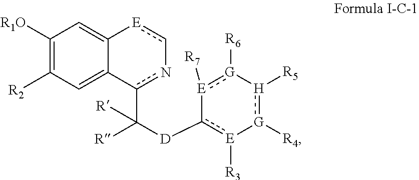

wherein
D is selected from CR′R″, NR′, and O,
R′ and R″ are independently selected from hydrogen, hydroxyl, halogen, amine, alkylamine, C₁-C₆ alkyl, C₁-C₆ alkyl halide, C₁-C₆ alkoxy, or R′and R″ combine together with the atom to which they are attached form a carbonyl;
R′ is selected from hydrogen and C₁-C₆ alkyl;
R² is selected from hydrogen, hydroxyl, C₁-C₆ alkyl, and C₁-C₆alkoxy; and
R³ to R⁷ are independently selected from hydrogen, hydroxyl, C₁-C₆ alkyl, C₁-C₆ alkyl halide,

C₁-Ccalkoxy, and C₁-C₆ alkylamine or R³ and R⁴ or R⁴ and R⁵ or R⁶ and R⁶ or R⁶ and R⁷ combine together with the atoms to which they are attached form a C₅-C₈ aryl or heteroaryl, or C₅-C₈ cycloalkenyl or heterocycloalkenyl,
wherein R³ to R⁷ are independently unsubstituted or substituted with hydroxyl, halogen, C₁-C₃ alkyl, alkenyl, or C₁-C₃ alkyl halide.

In certain embodiments of Formula I, the compound can be represented by a structure having the Formula I′-C-1:

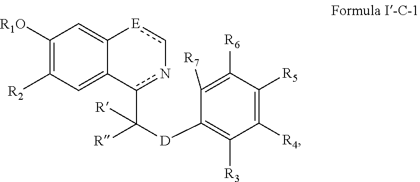

wherein
D is selected from CR′R″, NR′, and O,
R′ and R″ are independently selected from hydrogen, hydroxyl, halogen, amine, alkylamine, C₁-C₆ alkyl, C₁-C₆ alkyl halide, C₁-C₆ alkoxy, or R′ and R″ combine together with the atom to which they are attached form a carbonyl;
R¹ is selected from hydrogen and C₁-C₆ alkyl;
R² is selected from hydrogen, hydroxyl, C₁-C₆ alkyl, and C₁-C₆ alkoxy; and
R³ to R⁷ are independently selected from hydrogen, hydroxyl, C₁-C₆ alkyl, C₁-C₆ alkyl halide, alkoxy, and C₁-C₆ alkylamine or R³ and R⁴ or R⁴ and R⁵ or R⁵ and R⁶ or R⁶ and
R⁷ combine together with the atoms to which they are attached form a C₅-C₈ aryl or heteroaryl, or C₁-C₈ cycloalkenyl or heterocycloalkenyl,
wherein R³ to R⁷ are independently unsubstituted or substituted with hydroxyl, halogen, C₁-C₃ alkyl, C₁-C₃ alkenyl, or C₁-C₃ alkyl halide.

In certain embodiments, the compound can be represented by a structure having the Formula II:

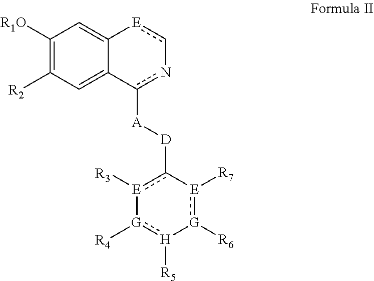

wherein
A and D are independently present or absent and are independently selected from CRR, and O, wherein R′ and R″ are independently for each occurrence selected from hydrogen, hydroxyl, halogen, amine, alkylamine, C₁-C₆ alkyl, C₁-C₆ alkyl halide, C₁-C₆ alkoxy, or R and R combine together with the atom to which they are attached form a carbonyl group;
E, G, and H can be independently selected from C, N′, O, and S;
R¹ and R² are independently selected from hydrogen, C₁-C₆ alkyl, C₁-C₆ alkyl halide, C₁-C₆ alkoxy, and C l-C₆ alkylamine;
R³ to R⁷ are independently selected from hydrogen, hydroxyl, C₁-C₆ alkyl, C₁-C₆ alkyl halide, C₁-C₆ alkoxy, and C₁-C₆ alkylamine or R.' and R⁴ or R⁴ and R⁵ or R⁵ and R⁶ or R⁶ and R⁷ combine together with the atoms to which they are attached form a C₅-C₈ aryl or heteroaryl, or C₅-C₈ cycloalkenyl or heterocycloalkenyl; wherein R³ to R⁷ are independently unsubstituted or substituted with hydroxyl, halogen, C₁-C₃ alkyl, C₁-C₃ alkenyl, or C₁-C₃ alkyl halide; and [symbol] represents a bond and is independently for each occurrence absent or present.

In some embodiments of the formulas described herein (including Formulas I, I′, I-A to I-C, I′-A to I′-C, I-A-1 to I-C-1, I′-A-1 to I′-C-1, and II), A can be selected from CR′R″ and O, wherein R′ and R″ are as defined herein. In some embodiments of the formulas described herein, D is selected from CRR and O, wherein R′ and R″ are as defined herein. In some embodiments of the formulas described herein, A and D can both be CR′R″. In some embodiments of the formulas described herein, A can be CR′R″ and D is O. In some embodiments of the formulas described herein, A is present and D is absent.

In some embodiments of the formulas described herein, R′ and R″ can be independently for each occurrence selected from hydrogen, hydroxyl, C₁-C₆ alkyl, or R′ and R″ combine together with the atom to which they are attached can form a carbonyl. For example, R′ and R″ can be hydrogen. In some examples, R′ can be hydrogen and at least one occurrence of R″ can be hydroxyl. In other examples, at least one occurrence of R′ and R″ combine together with the atom to which they are attached form a carbonyl.

In some embodiments of the formulas described herein, R¹ can be selected from a C₁-C₆ alkyl. For example, R¹ can be selected from a C₁-C₂ alkyl such as methyl or ethyl. In some examples, R¹ can be methyl.

In some embodiments of the formulas described herein, R² can be independently selected from a C₁-C₆ alkoxy. For example, R² is selected from a C₁-C₂ alkoxy such as methoxy or ethoxy. In some examples, R² can be methoxy. In other examples, R² can be hydrogen,

In some embodiments of the formulas described herein, R³, R⁴, R⁵, R⁶, and R⁷ can be independently selected from hydrogen, hydroxyl, C₁-C₆ alkyl, C₁-C₆ alkyl halide, C₁-C₆ alkoxy, or R³ and R⁴ or R⁴ and combine together with the atoms to which they are attached form a C₅-C₈ aryl or heteroaryl, or C₅-C₈ cycloalkenyl or heterocycloalkenyl. For example, R³ to R⁷ can be independently unsubstituted or substituted with hydroxyl, halogen, C₁-C₃ alkyl, C₁-C₃ alkenyl, or C₁-C₃ alkyl halide. In some examples, R³ and. R⁷ are hydrogen. In some examples, R⁴, R⁵, and R⁶ are independently selected from hydrogen, hydroxyl, C₁-C₆ alkyl, C₁-C₆ alkyl halide, or C₁-C₆ alkoxy. In further examples, R³ and R⁴ or R⁴ and R⁵ combine together with the atoms to which they are attached form a C₆ aryl, a C₆ heteroaryl, or a C₅ heterocycloalkenyl.

In certain embodiments of the formulas described herein, the compound can be represented by a structure below:

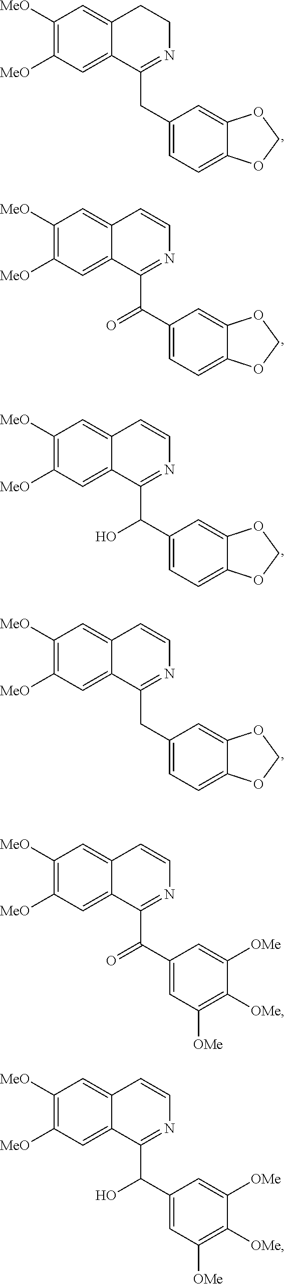 [SMILES: COc1cc2c(cc1OC)C(Cc1ccc3c(c1)OCO3)=NCC2 ; COc1cc2ccnc(C(=O)c3ccc4c(c3)OCO4)c2cc1OC ; COc1cc2ccnc(C(O)c3ccc4c(c3)OCO4)c2cc1OC ; COc1cc2ccnc(Cc3ccc4c(c3)OCO4)c2cc1OC ; COc1cc2ccnc(C(=O)c3cc(OC)c(OC)c(OC)c3)c2cc1OC ; COc1cc2ccnc(C(O)c3cc(OC)c(OC)c(OC)c3)c2cc1OC]

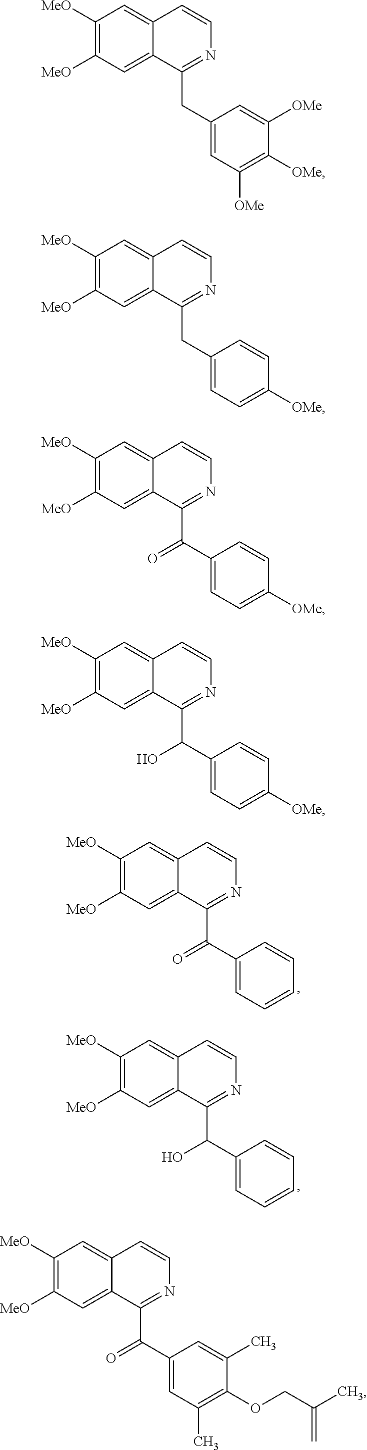

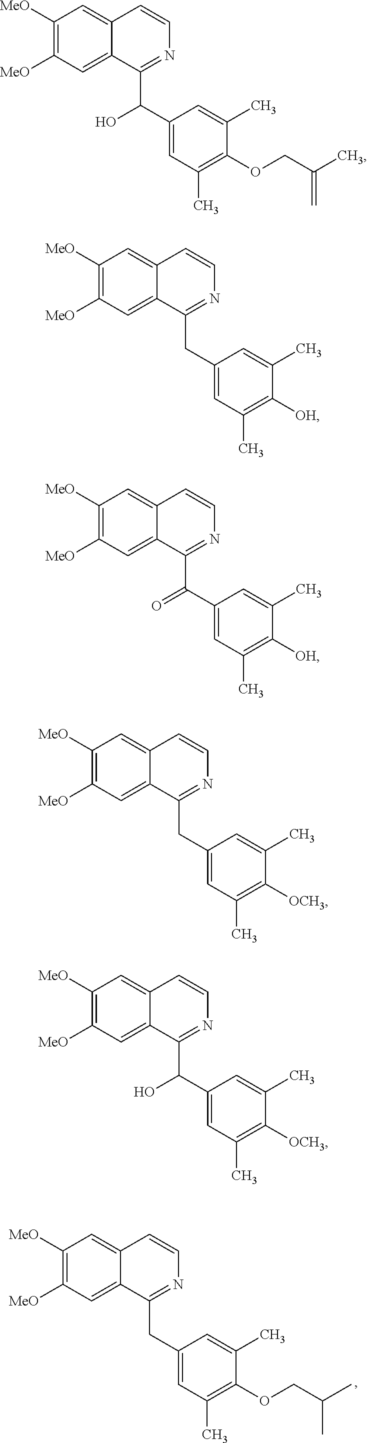

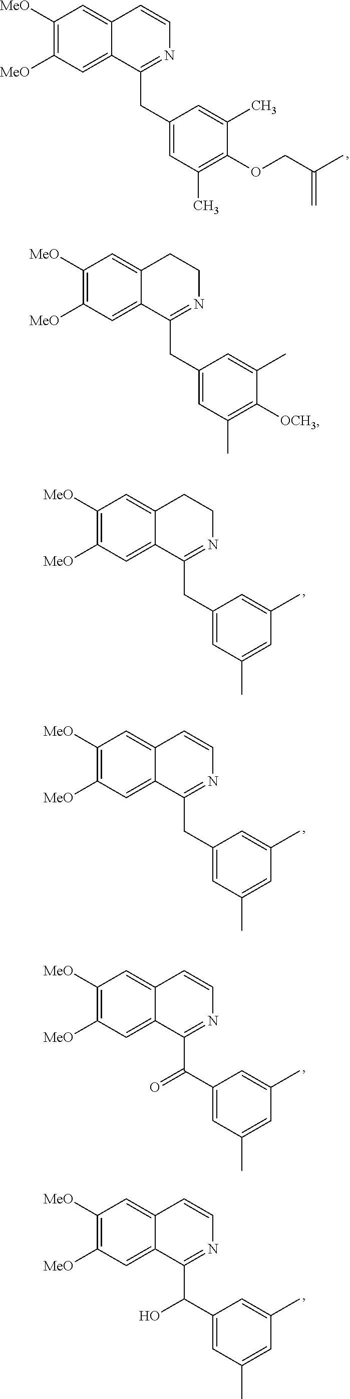 [SMILES: C=C(C)COc1c(C)cc(Cc2nccc3cc(OC)c(OC)cc23)cc1C ; COc1cc2c(cc1OC)C(Cc1cc(C)c(OC)c(C)c1)=NCC2 ; COc1cc2c(cc1OC)C(Cc1cc(C)cc(C)c1)=NCC2 ; COc1cc2ccnc(Cc3cc(C)cc(C)c3)c2cc1OC ; COc1cc2ccnc(C(=O)c3cc(C)cc(C)c3)c2cc1OC ; COc1cc2ccnc(C(O)c3cc(C)cc(C)c3)c2cc1OC]

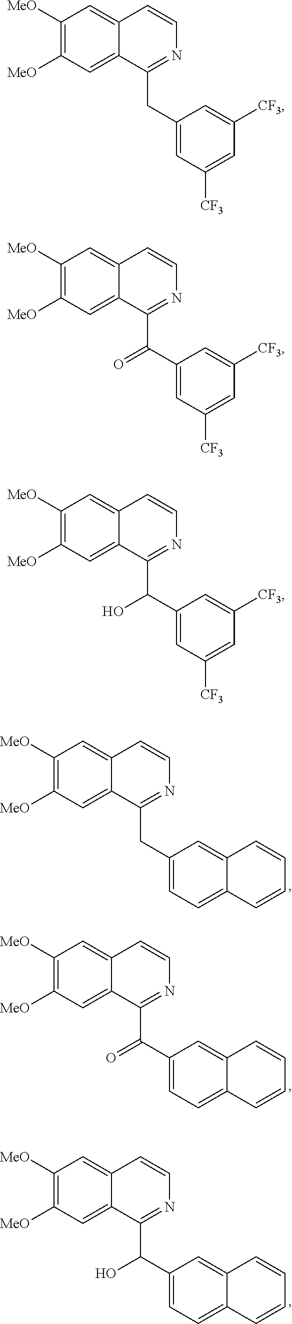 [SMILES: COc1cc2ccnc(Cc3cc(C(F)(F)F)cc(C(F)(F)F)c3)c2cc1OC ; COc1cc2ccnc(C(=O)c3cc(C(F)(F)F)cc(C(F)(F)F)c3)c2cc1OC ; COc1cc2ccnc(C(O)c3cc(C(F)(F)F)cc(C(F)(F)F)c3)c2cc1OC ; COc1cc2ccnc(Cc3ccc4ccccc4c3)c2cc1OC ; COc1cc2ccnc(C(=O)c3ccc4ccccc4c3)c2cc1OC ; COc1cc2ccnc(C(O)c3ccc4ccccc4c3)c2cc1OC]

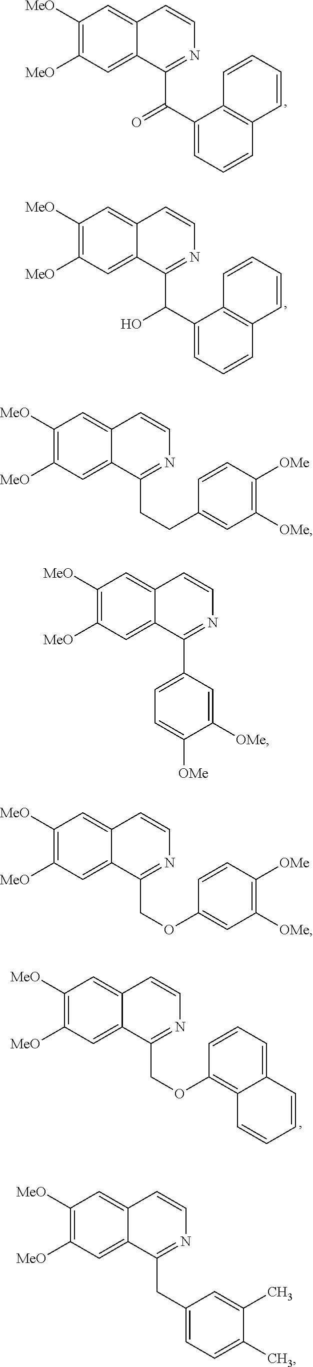

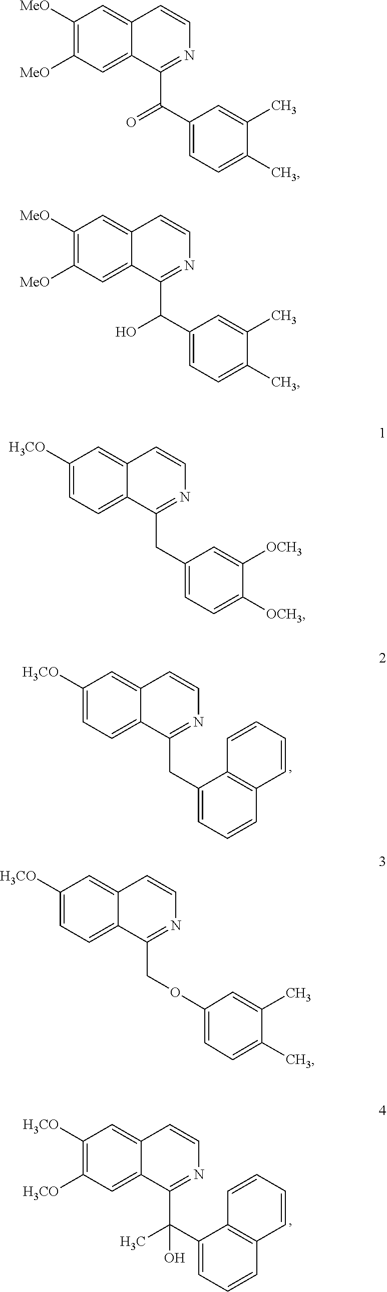 [SMILES: COc1cc2ccnc(C(=O)c3ccc(C)c(C)c3)c2cc1OC ; COc1cc2ccnc(C(O)c3ccc(C)c(C)c3)c2cc1OC ; COc1ccc2c(Cc3ccc(OC)c(OC)c3)nccc2c1 ; COc1ccc2c(Cc3cccc4ccccc34)nccc2c1 ; COc1ccc2c(COc3ccc(C)c(C)c3)nccc2c1 ; COc1cc2ccnc(C(C)(O)c3cccc4ccccc34)c2cc1OC]

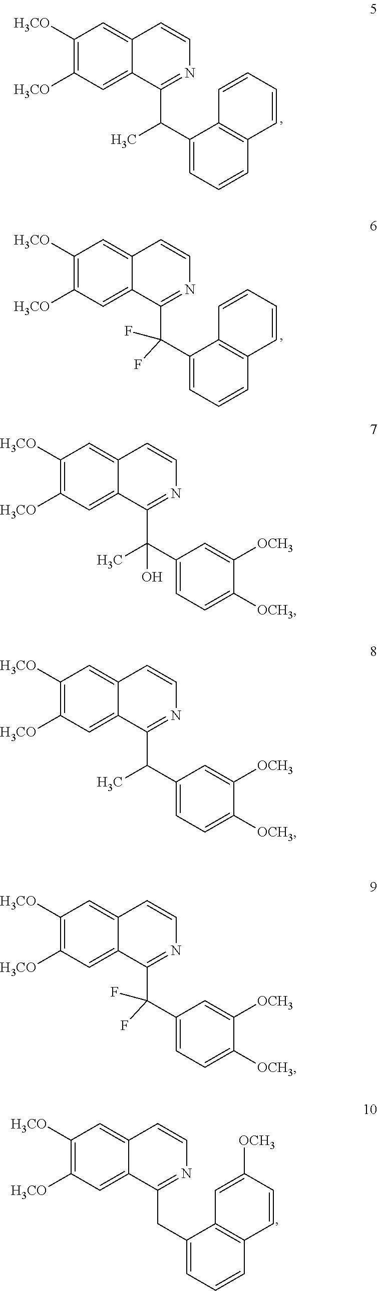 [SMILES: COc1cc2ccnc(C(C)c3cccc4ccccc34)c2cc1OC ; COc1cc2ccnc(C(F)(F)c3cccc4ccccc34)c2cc1OC ; COc1ccc(C(C)(O)c2nccc3cc(OC)c(OC)cc23)cc1OC ; COc1ccc(C(C)c2nccc3cc(OC)c(OC)cc23)cc1OC ; COc1ccc(C(F)(F)c2nccc3cc(OC)c(OC)cc23)cc1OC ; COc1ccc2cccc(Cc3nccc4cc(OC)c(OC)cc34)c2c1]

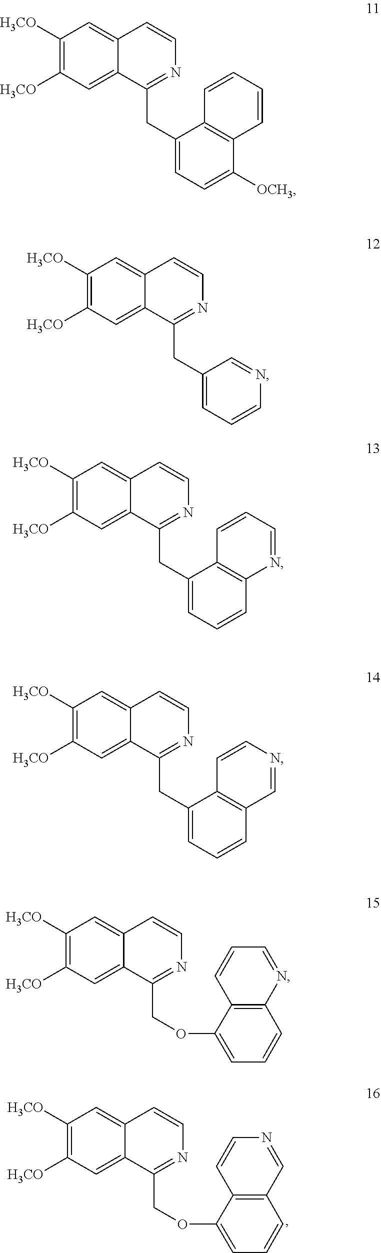 [SMILES: COc1cc2ccnc(Cc3ccc(OC)c4ccccc34)c2cc1OC ; COc1cc2ccnc(Cc3cccnc3)c2cc1OC ; COc1cc2ccnc(Cc3cccc4ncccc34)c2cc1OC ; COc1cc2ccnc(Cc3cccc4cnccc34)c2cc1OC ; COc1cc2ccnc(COc3cccc4ncccc34)c2cc1OC ; COc1cc2ccnc(COc3cccc4cnccc34)c2cc1OC]

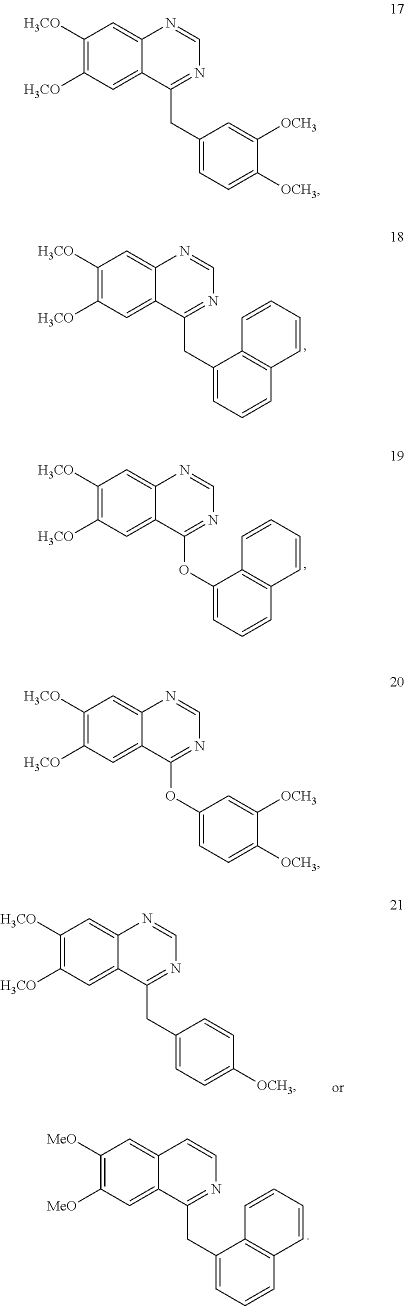 [SMILES: COc1ccc(Cc2ncnc3cc(OC)c(OC)cc23)cc1OC ; COc1cc2ncnc(Cc3cccc4ccccc34)c2cc1OC ; COc1cc2ncnc(Oc3cccc4ccccc34)c2cc1OC ; COc1ccc(Oc2ncnc3cc(OC)c(OC)cc23)cc1OC ; COc1ccc(Cc2ncnc3cc(OC)c(OC)cc23)cc1 ; COc1cc2ccnc(Cc3cccc4ccccc34)c2cc1OC]

Pharmaceutical Compositions

The disclosed compounds can be used therapeutically in combination with a pharmaceutically acceptable carrier. The carrier would naturally be selected to minimize any , degradation of the active ingredient and to minimize any adverse side effects in the subject, as would be well known to one of skill in the art.

The disclosed compounds may be in solution, suspension, incorporated into microparticles, liposomes, or cells, or formed into tablets, gels, or suppositories. Suitable carriers and their formulations are described in Remington: The Science and Practice of Pharmacy (22^{nd} ed.) eds. Loyd V. Alen, Jr., et al., Pharmaceutical Press, 2012. Typically, an approptiate amount of a pharmaceutically-acceptable salt is used in the formulation to render the formulation isotonic. Examples of the pharmaceutically-acceptable carrier include, but are not limited to, saline. Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered. Pharmaceutical carriers are known to those skilled in the art. These most typically would be standard carriers for administration of vaccines to humans, including solutions such as sterile water, saline, and buffered solutions at physiological pH. Pharmaceutical compositions may include carriers, thickeners, diluents, buffers, preservatives, surface active agents and the like in addition to the vaccine. Pharmaceutical compositions may also include one or more active ingredients such as antimicrobial agents, anti-inflammatory agents, anesthetics, and the like.

The disclosed compounds are preferably formulated for delivery via intranasal, intramuscular, subcutaneous, parenteral, transdermal or sublingual administration.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Parenteral administration of the disclosed compounds, if used, is generally characterized by injection. Injectables can be prepared in conventional forms, either as liquid solutions or suspensions, solid forms suitable for solution of suspension in liquid prior to injection, or as emulsions.

For an oral administration form, the disclosed compounds can be mixed with suitable additives, such as excipients, stabilizers or inert diluents, and brought by means of the customary methods into the suitable administration forms, such as tablets, coated tablets, hard capsules, aqueous, alcoholic, or oily solutions. Examples of suitable inert carriers are gum arabic, magnesia, magnesium carbonate, potassium phosphate, lactose, glucose, or starch, in particular, cornstarch. In this case, the preparation can be carried out both as dry and as moist granules. Suitable oily excipients or solvents are vegetable or animal oils, such as sunflower oil or cod liver oil. Suitable solvents for aqueous or alcoholic solutions are water, ethanol, sugar solutions, or mixtures thereof. Polyethylene glycols and polypropylene glycols are also useful as fiirther auxiliaries for other administration forms. As immediate release tablets, these compositions may contain microcrystalline cellulose, dicalcium phosphate, starch, magnesium stearate and lactose and/or other excipients, binders, extenders, di sintegrants, diluents and lubricants known in the art.

When administered by nasal aerosol or inhalation, the disclosed compounds may be prepared according to techniques well-known in the art of pharmaceutical formulation and may be prepared as solutions in saline, employing benzyl alcohol or other suitable preservatives, absorption promoters to enhance bioavailability, fluorocarbons, and/or other sotubili zing or dispersing agents known in the art. Suitable pharmaceutical formulations for administration in the form of aerosols or sprays are, for example, solutions, suspensions or emulsions of the compounds of the disclosure or their physiologically tolerable salts in a pharmaceutically acceptable solvent, such as ethanol or water, or a mixture of such solvents. If required, the formulation may additionally contain other pharmaceutical auxiliaries such as surfactants, emulsifiers and stabilizers as well as a propellant.

For subcutaneous or intravenous administration, the disclosed compounds, if desired with the substances customary therefore such as solubilizers, emulsifiers or further auxiliaries are brought into solution, suspension, or emulsion. The disclosed compounds may also be lyophilized and the lyophilizates obtained used, for example, for the production of injection or infusion preparations. Suitable solvents are, for example, water, physiological saline solution tri or alcohols, e.g. ethanol, propanol, glycerol, sugar solutions such as glucose or mannitol solutions, or mixtures of the various solvents mentioned. The injectable solutions or suspensions may be formulated according to known art; using suitable non-toxic, parenterally-acceptable diluents or solvents, such as mannitol, 1.3-butanediol, water, Ringer's solution or isotonic sodium chloride solution, or suitable dispersing or wetting and suspending agents, such as sterile, bland, fixed oils, including synthetic mono- or diglycerides, and fatty acids, including oleic acid.

When rectally administered in the form of suppositories, the formulations may be prepared by mixing the compounds with a suitable non-irritating excipient, such as cocoa butter, synthetic glyceride esters or polyethylene glycols, which are solid at ordinary temperatures, but liquefy and/or dissolve in the rectal cavity to release the drug.

In certain embodiments, it is contemplated that compositions comprising the disclosed compounds can be extended release formulations. Typical extended release formations utilize an enteric coating. Typically, a barrier is applied to oral medication that controls the location in the digestive system where it is absorbed. Enteric coatings prevent release of medication before it reaches the small intestine. Enteric coatings may contain polymers of polysaccharides, such as maltodextrin, xa.nthan, scleroglucan dextran, starch, alginates, pullulan, hyaloronic acid, chitin, chitosan and the like; other natural polymers, such as proteins (albumin, gelatin etc.), poly-L,-lysine; sodium poly(acrylic acid); poly(hydroxyalkylmethacrylates) (for example poly(hydroxyethylmethacrylate)); carboxypolymethylene (for example Carboporn; carbomer; polyvinylpyrrolidone; gums, such as guar gum, gum arabic, gum karaya, gum Bhatti, locust bean gum, tamarind gum, gellan gum, gum tragacanth, agar, pectin, gluten and the like; polyvinyl alcohol); ethylene vinyl alcohol; polyethylene glycol (PEG); and cellulose ethers, such as hydroxymethylcellulose (HMC), hydroxyethylcellulose (HEC), hydroxypropylcellulose (HPC), methylcellulose (MC), ethylcellulose (EC), carboxyethylcellulose (CEC), ethylhydroxyethylcellulose (EHEC), carboxymethylhydroxyethylcellulose (CMHEC), hydroxypropylinethyl-cellulose (HPMC), hydroxypropylethylcellulose (HPEC) and sodium carboxymethylcellulose (Na-CMC); as well as copolymers and/or (simple) mixtures of any of the above polymers. Certain of the above-mentioned polymers may further be crosslinked by way of standard techniques.

The choice of polymer will be determined by the nature of the active ingredient/drug, that is employed in the composition of the disclosure as well as the desired rate of release. In particular, it will be appreciated by the skilled person, for example in the case of HPMC, that a higher molecular weight will, in general, provide a slower rate of release of drug from the composition. Furthermore, in the case of EIPMC, different degrees of substitution of methoxyl groups and hydroxypropoxyl groups will give rise to changes in the rate of release of drug from the composition. In this respect, and as stated above, it may be desirable to provide compositions of the disclosure in the form of coatings in which the polymer carrier is provided by way of a blend of two or more polymers of, for example, different molecular weights in order to produce a particular required or desired release profile.

Microspheres of polylactide, polyglycolide, and their copolymers poly(lactide-co-glycolide) may be used to form sustained-release delivery systems. The disclosed compounds can be entrapped in the poly(lactide-co-glycolide) microsphere depot by a number of methods, including formation of a water-in-oil emulsion with water-borne compound and organic solvent-borne polymer (emulsion method), formation of a solid-in-oil suspension with solid compound dispersed in a solvent-based polymer solution (suspension method), or by dissolving the compound in a solvent-based polymer solution (dissolution method). One can attach polyethylene glycol) to compounds (PEGylation) to increase the in vivo half-life of circulating therapeutic proteins and decrease the chance of an immune response.

Liposomal suspensions (including liposomes targeted to viral antigens) may also be prepared by conventional methods to produce pharmaceutically acceptable carriers. This may be appropriate for the delivery of free nucleosides, acyl nucleosides or phosphate ester prodrug forms of the nucleoside compounds according to the present disclosure.

The exact amount of the compounds or compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein. For example, effective dosages and schedules for administering the compositions may be determined empirically, and making such determinations is within the skill in the art. The dosage ranges for the administration of the compositions are those large enough to produce the desired effect in which the symptoms disorder are affected. The dosage should not be so large as to cause adverse side effects; such as unwanted cross-reactions, anaphylactic reactions; and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counter indications. Dosage can vary, and can be administered in one or more dose administrations daily, for one or several days. Guidance can be found in the literature for appropriate dosages for given classes of pharmaceutical produas. A typical dosage of the disclosed vaccine used alone might range from about 1 μg/kg to up to 100 mg/kg of body weight or more per vaccination, such as 10 μg/kg to 50 mg/kg, or 50 μg/kg to 10 mg/kg, depending on the factors mentioned above.

Formulations for topical administration may include ointments, lotions, creams, gels, drops, suppositories, sprays, nanoparticles, liquids and powders. Conventional pharmaceutical carriers, aqueous, powder or oily bases, thickeners and the like may be necessary or desirable. Some of the disclosed compounds may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide; ammonium hydroxide, potassium hydroxide, and organic bases such as mono-, di-, trialkyl and aryl amines and substituted ethanolamines.

The pharmaceutical preparations of the disclosure are preferably in a unit dosage form, and may be suitably packaged, for example in a box, blister, vial, bottle, sachet, ampoule or in any other suitable single-dose or multi-dose holder or container (which may be properly labeled); optionally with one or more leaflets containing product information and/or instructions for use. Generally, such unit dosages will contain between 1 and 1000 mg, and usually between 5 and 500 mg, of the at least one compound of the disclosure, e.g., about 10, 50, 100, 200, 300 or 400 mg per unit dosage.

The disclosed compounds can also be used to supplement existing treatments. Therefore, the disclosed compositions can further include (or be administered in combination with) a second compound that can ameliorate, diminishing, reversing, treating or preventing cancer in a subject. For example, the disclosed compositions can further include (or be administered in combination with) one or more chemotherapeutic agents. In a specific embodiment, the disclosed compounds can be administered with (in combination in the same composition, in combination but in separate compositions, or sequentially) approved drugs for treating cancer.

The pharmaceutical compositions and formulations disclosed herein can be administered for prophylactic and/or therapeutic treatments. In therapeutic applications, compositions are administered to a subject already having a tumor.

The amount of pharmaceutical composition adequate to accomplish this is defined as a. “therapeutically effective dose.” The dosage schedule and amounts effective for this use, i.e., the “dosing regimen,” will depend upon a variety of factors, including the stage of the condition, the severity of the condition, the general state of the patient's health, the patient's physical status, age and the like. In calculating the dosage regimen for a patient, the mode of administration also is taken into consideration.

The dosage regimen also takes into consideration pharmacokinetics parameters well known in the art, i.e., the active agents' rate of absorption, bioavai lability, metabolism, clearance, and the like (see, e.g., Hidalgo-Aragones (1996) J. Steroid Biochem. Mol. Biol. 58:611-617; (ironing (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacal. 24:103-108; the latest Remington's, supra). The state of the art allows the clinician to determine the dosage regimen for each individual patient, active agent and disease or condition treated. Guidelines provided for similar compositions used as pharmaceuticals can be used as guidance to determine the dosage regiment, i.e., dose schedule and dosage levels, administered practicing the methods of the invention are correct and appropriate.

Methods of Use

Disclosed are methods for treating cancer in a subject. The method can include administering the compound or compositions disclosed herein in combination with an. immunotherapy. In some embodiments, the method for treating cancer in a subject comprises: administering to the subject a therapeutically effective amount of one or more compounds, or pharmaceutical composition disclosed herein to inhibit mitochondrial oxygen consumption in a cancer cell; and administering an immunotherapy to treat the cancer. The disclosure also provides methods for treating or ameliorating at least one symptom or indication, or inhibiting the growth of a locally advanced, surgically undesirable, or metastatic malignant melanoma in a subject. In certain embodiments, the disclosure provides methods for inhibiting mitochondrial oxygen consumption in a cancer cell. In specific embodiments, the disclosure provides methods for treating or ameliorating at least one symptom or indication, or inhibiting the growth of hypoxic tumors. Hypoxic tumors exists because the supply of oxygen is insufficient to meet the metabolic demand of the tumor. Disclosed are also methods of treating hypoxic tumors in a subject. The method can include administering the compound or compositions disclosed herein in combination with an immunotherapy.

The methods can comprise administering to a subject in need thereof an effective amount of one or more compounds or composition disclosed herein. The compound or composition can be in an effective amount to reduce oxygen consumption in the tumor cells. In some embodiments, the compounds and compositions can be in an effective amount to inhibit mitochondrial functions in the tumor cell. In some embodiments, the compounds or compositions disclosed herein can be in an effective amount to inhibit complex I of the mitochondrial respiratory chain. In some embodiments, the compounds or compositions are not PDE10A inhibitors. In some examples, the compounds and compositions for treating or reducing tumor hypoxia includes papaverine.

In certain embodiments, the compound or compositions disclosed herein can be administered to a subject in need thereof, each dose comprising 0.1-10 mg/kg (e.g., 0.3 mg/kg; 1 mg/kg, 3 mg/kg, or 10 mg/kg) of the subject's body weight. In certain other embodiments, each dose comprises 20-600 mg of the compound, e.g., 50 mg, 100 mg, 150 mg, 200 mg, 250 mg, 300 mg, 400 mg, or 500 mg of the compound.

As used herein ; the term “immunotherapy”, refers to an agent that modulates the immune system. In some embodiments, an immunotherapy can increase the expression and/or activity of a regulator of the immune system. In some embodiments, an immunotherapy can decrease the expression and/or activity of a regulator of the immune system. In some embodiments, an immunotherapy can recruit and/or enhance the activity of an immune cell. In some embodiments, an immunotherapy can be an anti-cancer immunotherapy.

In some embodiments, the immunotherapy is a cellular immunotherapy (e.g., adoptive T-cell therapy, dendritic cell therapy, natural killer cell therapy). In some embodiments, the cellular immunotherapy is sipuleucel-T (APCSOI5; Proyenge™; Plosker (2011) Drugs 71(1): 101-108). In some embodiments, the cellular immunotherapy includes cells that express a chimeric antigen receptor (CAR). In some embodiments, the cellular immunotherapy is a CAR-T cell therapy. In some embodiments, the CAR-T cell therapy is tisagenlecleucel (Kymriah™).

In some embodiments, the immunotherapy is an antibody therapy (e.g., a monoclonal antibody, a conjugated antibody). In some embodiments, the antibody therapy is bevacizumab (Mvasti™, AyastinO), trastuzumab (Herceptin®), avelumab (Bavencio®), rituximab (MabThera™, Rituxan®). edrecolomab (Panorex), daratumuab (Darzalexg), olaratumab (Lartruvo®), ofatumumab (Arzerra®), alemtuzumab (Campath®), cetuximab (Erbitux®), oregovornab, pembrolizutnab (Keytruda(®), dinutiximab (Unituxin®), obinutuzumab (Gazyva®), tremelimumab (CP-675,206), ramucirumab (Cyramza®), ublituximab (TG-1101), panitumumab (Vectibix®), elotuzumab (Empliciti™), avelumab (Bavencio®), necitumumab (Portrazza™), cirmtuzumab (UC-961), ibritumomab (Zevalinig), isatuximab (SAR650984), nitnotuzumab, fresolimumab (GC₁₀₀₈), lirilumab (INN), mogamulizumab (Poteligeo®), ficlatuzumab (AV-299), denosumab (Xgeva®), ganitumab, urelumab, pidilizumab or amatuximab.

In some embodiments, the immunotherapy is an antibody-drug conjugate. In some embodiments, the antibody-drug conjugate is g.emtuzumab ozogamicin (Mylotarg™), inotuzumab ozogamicin (Besponsa®), brentuximab vedotin (Adcetris®), ado-trastuzumab emtansine (TDM-1; Kadcylag), mirvetuximab soravtansine (IMGN853) or anetumab ravtansine

In some embodiments, the immunotherapy includes blinatumomab (AMG103; Blincyto®) or midostaurin (Rydapt).

In some embodiments, the immunotherapy includes a toxin. In some embodiments, the immunotherapy is denileukin diftitox (Ontak®).

In some embodiments, the immunotherapy is a cytokine therapy. In some embodiments, the cytokine therapy is an interleukin 2 (IL-2) therapy; an interferon alpha (ENO therapy, a granulocyte colony stimulating factor (G-CSF) therapy, an interleukin 12 (IL-12) therapy, an interleukin 15 (IL-15) therapy, an interleukin 7 (IL-7) therapy or an erythropoietin-alpha (EPO) therapy. In some embodiments, the IL-2 therapy is aidesleukin (Proleukin®). In some embodiments, the IFNα therapy is interferon alfa-2b (e.g., IntronA®) or interferon alfa-2a (e.g., Roferon-A®). In some embodiments, the (3-CSF therapy is filgrastim (Neupogen®).

In some embodiments, the immunotherapy is an immune checkpoint inhibitor. In some embodiments, the immunotherapy includes one or more immune checkpoint inhibitors. In some embodiments, the immune checkpoint inhibitor is a CTLA-4 inhibitor, a PD-1 inhibitor or a PD-Ll inhibitor. In some embodiments, the CTLA-4 inhibitor is ipilimumab (Yervoy®) iii or tremelimumab (CP-675,206). In some embodiments, the PD-1 inhibitor is pernbrolizurnab (Keytrudag) or nivolurnab (Opdivo®). In some embodiments, the PD-LI inhibitor is atezolizumab (Tecentrigg), avelumab (Bavencio®) or durvalumab (Imfinzim).

In some embodiments, the immunotherapy is mRNA-based immunotherapy. In some embodiments, the mRNA-based immunotherapy is CV9104 (see, e.g., Rausch et al. (2014) Human Vaccin Immunother 10(11): 3146-52; and Kubler et al. (2015) J. Immunother Cancer 3:26).

In some embodiments, the immunotherapy is bacillus Calmette-Guerin (B(G) therapy.

In some embodiments, the immunotherapy is an oncolytic virus therapy. In some embodiments, the oncolytic virus therapy is talimogene aiherparepvec (T-VEC; Imlygic®).

In some embodiments, the immunotherapy is a cancer vaccine. In some embodiments, the cancer vaccine is a human papillomavirus (HPV) vaccine. In some embodiments, the HPV vaccine is a recombinant human papillomavinis vaccine [types 6, 11, 16, and 18] (Gardasil®); a recombinant human papillomavirus vaccine [types 6, 11, 16, 18, 31, 33, 45, 52, and 58] (Gardasil9®); or a recombinant human papillomavirus vaccine [types 16 and 18] (Cervarix®). In some embodiments, the cancer vaccine is a hepatitis B virus (HBV) vaccine. In some embodiments, the HBV vaccine is Engerix-B®, Recombivax HB® or GS-4774 (GI-13020 or Tarmogen®). In some embodiments, the cancer vaccine is a combination Hepatitis A and Hepatitis B vaccine (e.g., Twinrix®) or a combination diphtheria, tetanus, pertussis, hepatitis B virus, and poliomyelitis vaccine (e.g., Pediarix®). In some embodiments, the cancer vaccine is dasiprotimut-T (BiovaxiD®), an HSPPC-96 vaccine (e.g., Oncophage®), GVAX, ADXS11-001, ALVAC-CEA, rilimogene gaivacirepvec/rilimogene glafolivec (PROSTVAC CDX-110 (Rindopepimut®), Cirna.Vax-EGF, lapuleucel-T (APC₈₀₂₄; Neuvenge™), GRNVAC1, GRNVAC₂, GRN-1201, hepcortespenlisimut-L (Repko-V5), a dendritic cell vaccine (e.g., DCVax-L®, ICT-107), SCIB1, BMT CTN 1401, PrCa VBIR, PANVAC, a prostate cancer vaccine (e.g., ProstAtak®), DPX-Survivac, or viagenpumatucel-L (HS-110).

In some embodiments, the immunotherapy is a peptide vaccine. In some embodiments, the peptide vaccine is nelipepiraut-S (E75) (NeuVax™), IMA901, or SurVaxM (SVN53-67). In some embodiments, the cancer vaccine is an immunogenic personal neoantigen vaccine (see, e.g., Ott et al. (2017) Nature 547: 217-221; Sahin et al. (2017) Nature 547: 222-226), In some embodiments, the cancer vaccine is RGSH4K, or NEO-PV-01.

In some embodiments, the cancer vaccine is a DNA-based vaccine. In some embodiments, the DNA-based vaccine is a mammaglobin-A DNA vaccine (see, e.g., Kim et al, (2016) Oncolinmunology 5(2): e1069940).

In certain embodiments, the cancer or tumor is a solid tumor or malignancy. The methods described herein can cause a therapeutic injury resulting in the reduction of at least one of surface area, the depth, and the amount of the tissue affected by the cancerous condition. In certain embodiments, the compounds and compositions can be used in the treatment of cancer of the bile duct, bone, bladder, head and neck, kidney, liver, gastrointestinal tissue, esophagus, ovary, endometrium, pancreas, skin, testes, thyroid, uterus, cervix and vulva, and or leukemias (including acute lymphoblastic leukemia (ALL), acute myeloid leukemia (AML), chordoma, chronic lymphocytic leukemia (CLL), and chronic myelogenous leukemia (CML)), multiple myeloma and lymphomas. In specific embodiments, the compounds and compositions can be used in the treatment of lung cancer, anal cancer, colorectal cancer, prostate cancer, melanoma, appendix cancer, renal cancer, skin cancer, testicular cancer, ovarian cancer, breast cancer, endometrial cancer, kidney cancer, gastric cancer, sarcomas, bladder cancer, brain cancer, cervical cancer, gastrointestinal cancer, genitourinary cancer, esophageal cancer, pancreatic cancer, bile duct cancer, colon cancer, cholangiocarcinoma, bronchial tumors, endometrial cancer, liver cancer, uterine cancer, bone cancer, stomach cancer, salivary gland cancer, head and neck cancers, tumors of the central nervous system and their metastases, and also for the treatment of glioblastomas and myeloma. In some specific embodiments, the cancer is lung cancer. In some specific embodiments, the cancer is breast cancer. In some specific embodiments, the cancer is colorectal cancer. In some specific embodiments, the cancer is pancreatic cancer. In some specific embodiments, the cancer is cervical cancer.

In some embodiments, compounds and compositions disclosed herein could be used in the clinic in combination with an immunotherapy, in combination with immunotherapy, and an additional chemotherapy agent, in combination with immunotherapy, an additional chemotherapy agent, and irradiation Such chemotherapy agent can include one or more of the following categories of anti-tumor agents:
(i) antiproliferative/antineoplastic drugs and combinations thereof, as used in medical oncology, such as alkylating agents (for example cis-platin, carboplatin, cyclophosphamide, nitrogen mustard, melphalan, chlorambucil, busulfan and nitrosoureas); antimetabolites (for example antifolates such as fluoropyrimidines like 5-fluorouracil and gemcitabine, tegafur, raltitrexed, methotrexate, cytosine arabinoside and hydroxyurea); antitumour antibiotics (for example anthracyclines like adriamycin, bleomycin, doxorubicin, daunomycin, epintbicin, idarubicin, mitomycin-C, dactinomycin and mithramycin); antimitotic agents (for example vinca alkaloids like vincristine, vinblastine, vindesine and vinorelbine and toxoids like taxol and taxotere); and topoisomerase inhibitors (for example epipodophyllotoxins like etoposide and teniposide, amsacrine, topotecan and camptothecin); and proteosome inhibitors (for example bortezomib [Velcade®]); and the agent anegrilide [Agiylin®]; and the agent alpha-interferon;
(ii) cytostatic agents such as anti-estrogens (for example tamoxifen, toremifene, raloxifene, droloxifene and iodoxyfene), oestrogen receptor down regulators (for example fUlvestrant), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;
(iii) agents that inhibit cancer cell invasion (for example metalloproteinase inhibitors like marimastat and inhibitors of urokinase plasminogen activator receptor function);
(iv) inhibitors of growth factor function; for example such inhibitors include growth factor antibodies, growth factor receptor antibodies (for example the anti-erbb2 antibody trastuzumab [Herceptin™] and the anti-erbbl antibody cetuximab), farnesyl transferase inhibitors, tyrosine kinase inhibitors and serine/threonine kinase inhibitors, for example inhibitors of the epidermal growth factor family (for example EGFR family tyrosine kinase inhibitors such as: N-(3-chloro-4-fluorophenyl)-7-methoxy-6-(3-morpholinopropoxy)quinazolin-4-amine (gefitinib), N-(3-ethynylphenyl)-6,7-bis(2-methoxyethoxy)quinazolin-4-amine (erlotinib), and 6-acrylamido-N-(3-chloro-4-fluorophenyl)-7-(3-morpholinopropoxy)quinazolin-4-amine (CI 1033), for example inhibitors of the platelet-derived growth factor family and for example inhibitors of the hepatocyte growth factor family, for example inhibitors or phosphotidylinositol 3-kinase (MK) and for example inhibitors of mitogen activated protein kinase kinase (MEK1/2) and for example inhibitors of protein kinase B (PKB/Akt), for example inhibitors of Src tyrosine kinase family and/or Abelson (Abi) tyrosine kinase family such as dasatinib (BMS-354825) and imatinib mesylate (Gleevec™); and any agents that modify STAT signalling;
(v) antiangiogenic agents such as those which inhibit the effects of vascular endothelial growth factor, (for example the anti-vascular endothelial cell growth factor antibody bevacizumab [Avastin™]) and compounds that work by other mechanisms (for example its linomide, inhibitors of integrin ocvβ3 function and angiostatin);
(vi) vascular damaging agents such as Combretastatin A4;
(vii) antisense therapies, for example those which are directed to the targets listed above, such as an anti-ras anti sense; and
(viii) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy.

Combination treatment with an additional chemotherapy agent can be achieved by way of the simultaneous, sequential or separate dosing of the individual components of the treatment. Such combination products employ the compounds or compositions of this disclosure, or pharmaceutically acceptable salts thereof, within the dosage range described hereinbefore and the other pharmaceutically-active agent within its approved dosage range.

Radiation therapy can be delivered by a machine placed outside the subject's body (external-beam radiation therapy), or by a source placed inside a subject's body (internal radiation therapy or brachytherapy), or through systemic radioisotopes delivered intravenously or orally (systemic radioisotope therapy). “XRT” means using ionizing radiation to kill cancer cells, generally as part of anti-cancer therapy. X-rays, gamma rays or charged particles (e.g., protons or electrons) are used to generate ionizing radiation. Radiation therapy can be planned and administered in conjunction with imaging-based techniques such a computed tomography (CT), magnetic resonance imaging (MRI) to accurately determine the dose and location of radiation to be administered. In various embodiments, radiation therapy can be selected from the group consisting of total all-body radiation therapy, conventional external beam radiation therapy, stereotactic radiosurgery, stereotactic body radiation therapy, 3-D conformal radiation therapy, intensity-modulated radiation therapy, image-guided radiation therapy, tomotherapy, brachytherapy, and systemic radiation therapy. In certain embodiments, the radiation therapy comprises hypofractionated radiation therapy.

In certain embodiments, the method can include administering one or more doses in a treatment cycle. For example, the method can comprise administering to a. subject in need thereof at least one treatment cycle, wherein the at least one treatment cycle comprises 1-10 doses of a compound or composition disclosed herein and optionally one or more doses of radiation therapy. In certain embodiments, 2-12 treatment cycles are administered to a subject in need thereof.

In some embodiments, the one or more compounds, or the composition comprising the one or more compounds can be administered prior to, during, or after the immunotherapy is administered. In some embodiments, the one or more compounds, or the composition comprising the one or more compounds can be administered prior to, during, and after the immunotherapy is administered. In some embodiments, the one or more compounds, or the composition comprising the one or more compounds can be administered prior to and during the immunotherapy is administered. In some embodiments, the one or more compounds, or the composition comprising the one or more compounds can be administered prior to and after the immunotherapy is administered. In some embodiments, the one or more compounds, or the composition comprising the one or more compounds can be administered prior to the immunotherapy is administered. In some embodiments, the one or more compounds, or the composition comprising the one or more compounds can be administered during the immunotherapy is administered. In some embodiments ; the one or more compounds, or the composition comprising the one or more compounds can be administered after the immunotherapy is administered.

In some embodiments, the one or more compounds, or the composition comprising the one or more compounds can be administered at least 5 minutes (e.g., at least 30 minutes, at least 1 hour, at least 2 hours, at least 4 hours, at least 6 hours, at least 8 hours, at least 10 hours, at least 12 hours, at least 16 hours, at least 18 hours, at least 20 hours, or at least 24 hours) prior to administering the immunotherapy.

In some embodiments, the one or more compounds, or the composition comprising the one or more compounds can be administered 2 days or less. ; (e.g., 24 hours or less, 20 hours or less, 18 hours or less, 16 hours or less, 12 hours or less, 10 hours or less, 8 hours or less, 6 hours or less, 4 hours or less, 2 hours or less, 1 hour or less, or 30 minutes or less) prior to administering the immunotherapy.

In some embodiments, the one or more compounds, or the composition comprising the one or more compounds can be administered within 5 minutes to 2 days (e.g., within 30 minutes to 24 hours, preferably within 30 minutes to 90 minutes, within 30 minutes to 2 hours, within 30 minutes to 4 hours, within 30 minutes to 6 hours, within 30 minutes to 8 hours, within 30 minutes to 10 hours, within 30 minutes to 12 hours, within 30 minutes to 16 hours, within 30 minutes to 20 hours, within 30 minutes to 22 hours, within 4 hours to 16 hours, within 6 hours to 12 hours, within 6 hours to 24 hours, or within 4 hours to 12 hours) prior to administering the immunotherapy.

In some embodiments, the one or more compounds, or the composition comprising the one or more compounds can be administered at least 5 minutes (e.g., at least 30 minutes, at least 1 hour, at least 2 hours, at least 4 hours, at least 6 hours, at least 8 hours, at least 10 hours, at least 12 hours, at least 16 hours, at least 18 hours, at least 20 hours, at least 1 day, at least 2 days, at least 3 days, at least 4 days, at least 5 days, at least 6 days, at least 7 days, at least 1 week, at least 2 weeks, at least 3 weeks, at least 1 month, at least 2 months, at least 4 months, or at least 5 months) after administering the immunotherapy.

In some embodiments, the one or more compounds, or the composition comprising the one or more compounds can be administered 6 months or less, (e.g., 5 months or less, 4 months of less, 3 months or less, 2 months or less, 1 month or less, 3 weeks or less, 2 weeks or less, 1 week or less, 7 days or less, 6 days or less, 5 days or less, 4 days or less, 3 days or less, 2 days or less, 24 hours or less, 20 hours or less, 18 hours or less, 16 hours or less, 12 hours or less, 10 hours or less, 8 hours or less, 6 hours or less, 4 hours or less, 2 hours or less, 1 hour or less, 30 minutes or less, 25 minutes or less, 20 minutes or less, 15 minutes or less, or 10 minutes or less) after the immunotherapy is administered.

In some embodiments, the one or more compounds, or the composition comprising the one or more compounds can be administered within 5 minutes to 6 months (e.g., within 5 minutes to 24 hours, within 15 minutes to 24 hours. within 30 minutes to 24 hours, within 30 minutes to 90 minutes, within 30 minutes to 2 hours, within 30 minutes to 4 hours, within 30 minutes to 6 hours, within 30 minutes to 8 hours, within 30 minutes to 10 hours, within 30 minutes to 12 hours, within 30 minutes to 16 hours, within 30 minutes to 20 hours, within 30 minutes to 22 hours, within 1 hour to 16 hours, within 4 hours to 16 hours, within 6 hours to 12 hours, within 6 hours to 24 hours, within 1 hour to 5 hours, within 1 hour to 12 hours, within 4 hours to 12 hours, within 1 day to 1 week, within 1 day to 2 weeks, within 1 day to 3 weeks, within 1 day to 1 month, within 1 day to 2 months, within 1 day to 3 months, within 1 day to 4 months, within 1 day to 5 months, within 1 day to 6 months, within 1 week to 6 months, within 1 week to 1 month, within 1 week to 2 months, within 1 week to 3 months, within I week to 4 months, or within 1 week to 5 months) after administering the immunotherapy.

The methods can comprise administering to a subject with a cancer a therapeutically effective amount of a compound or composition disclosed herein prior to administering an immunotherapy, wherein the compound or composition can be administered on the same day as the immunotherapy is administered. In some embodiments, the compound or composition can be administered 6 hours or less, 5 hours or less, 4 hours or less, 3 hours or less, 2 hours or less, 1 hour or less, or 0.5 hours or less, prior to the and immunotherapy.

The methods can comprise administering to a subject with a solid tumor a therapeutically effective amount of a compound or composition disclosed herein and the immunotherapy prior to administering a radiation dose, wherein the compound or composition and the immunotherapy can be administered on the same day as the radiation is administered.

In some embodiments, the compound or composition and an immunotherapy can be administered 6 hours or less, 5 hours or less, 4 hours or less, 3 hours or less, 2 hours or less, hour or less, or 0.5 hours or less, prior to the radiation therapy.

In some embodiments the immunotherapy can be administered with less frequency than the one or more compounds or composition comprising the one or more compounds. In some embodiments, the immunotherapy and the one or more compounds or the composition comprising the one or more compounds can be administered every day. In some embodiments, the immunotherapy can be administered every week, every two weeks, every three weeks, every month, every two months, every three months, every four months, every 5 months, or every 6 months; and the one or more compounds or the composition comprising the one or more compounds can be administered every day. For example, in some embodiments, the immunotherapy can be administered every two weeks, and the one or more compounds or the composition comprising the one or more compounds can be administered every day.

In specific embodiments, the present disclosure provides methods for increased anti-tumor efficacy or increased tumor inhibition. In certain embodiments, the methods provide for increased tumor inhibition, e.g., by about 20%, more than 20%, more than 30%, more than 40% more than 50%, more than 60%, more than 70% or more than 80% as compared to a subject administered an immunotherapy absent a compound or compositions disclosed herein. The methods described herein are provided for treating or ameliorating at least one symptom or indication, or inhibiting the growth of cancer in a subject. In certain embodiments, the methods described herein are provided for treating or ameliorating at least one symptom or indication, or inhibiting the growth of hypoxic cancer in a subject. In certain embodiments, methods are provided for increasing the overall or progression-free survival of a patient with cancer. In some embodiments, the compounds and compositions are effective immune-sensitizers.

By way of non-limiting illustration, examples of certain embodiments of the present disclosure are given below.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the scope of the disclosure. Unless indicated otherwise, parts are parts by weight, temperature is in c or is at ambient temperature, and pressure is at or near atmospheric.

### Example 1

Background

Many human tumors contain regions of low oxygen tension, or hypoxia. Hypoxia exists because the supply of oxygen to the tumor does not meet the metabolic demand of the tissue. Tumor hypoxia exists as a mismatch between oxygen supply and demand, so we have taken an approach to address tumor hypoxia by decreasing the demand side rather than increasing the supply side. Mitochondria are the major sink for molecular oxygen in the cell, so we therefore identified novel mitochondrial inhibitors that could reduce oxygen consumption in hypoxic tissue and bring oxygen supply and demand into balance. To clinically translate this strategy, we first screened FDA-approved drugs for “off target” effects on the mitochondria and found papaverine (PPV), an ergot alkaloid and vasorelaxant first isolated in 1848 as an effective agent to reduce oxygen consumption^{(1,2)}. We have since made and tested 47 derivatives of papaverine, some of which have superior characteristics as immune-sensitizers.

PPV's activity as a phosphodiesterase 10A (PDEIOA) inhibitor is responsible for its activity as a vasorelaxant, but not for its effect inhibiting mitochondrial complex 1. Systemic vasorelaxation induced by PDE inhibition can also drop blood pressure and decrease blood flow to the tumor (vascular steal)^{(3,4)} that may limit activity of vascular agents as anti-hypoxia agents^{(5,6)}. We have therefore begun generating derivatives of PPV with reduced PDE inhibitory activity, while retaining mitochondrial inhibitory activity. Derivatives SMV32 and SMV37 retain mitochondrial activity, reduce tumor hypoxia, and increase radiation response of murine tumors. Initial studies show that SMV32 does not wash out of cells and so it has pharmacokinetic parameters superior to PPV as an immune-sensitizer⁽⁷⁾.

Several groups used the Eppendorf needle oxygen electrode to show that in human tumors pretreatment hypoxia is an independent prognostic indicator of poor patient outcome⁽⁸⁻¹⁰⁾. These studies clearly showed that a small fraction of very hypoxic cells in tumors could decreaseJocal control and reduce overall survival in patients treated with conventionally fractionated radiotherapy or immunotherapy⁽¹¹⁻¹⁴⁾. Clinical approaches designed to overcome hypoxia have had disappointing results. Strategies designed to deliver more oxygen⁽¹⁵⁾, deliver oxygen-mimetics⁽¹⁶⁾ or deliver drugs with preferential toxicity towards hypoxic cells⁽¹⁷⁾ have all failed in human trials⁽¹⁸⁻²¹⁾. These strategies are limited by the poor perfusion of the tumor. Tumor blood vessels have blind ends, breaks, and tortuous paths, all of which limit blood delivery and laminar blood flow and prevent oxygen delivery, even with increased systemic oxygen delivery⁽²²⁾. Not surprisingly, there is currently no FDA-approved hypoxia-directed anti-cancer clinical intervention.

Results and Discussion

Disclosed here are methods of enhancing the effectiveness of immunotherapy in the treatment of cancerous tissue. In a preferred mode, the method consists of delivery of one of the SMV compounds disclosed below in a dose of 0.1 to 10 mg/Kg (patient mass) given once or multiple times, made up in appropriate solution or suspension or emulsion, by IV injection, subcutaneous injection, dermal application or inhalation route either prior to, during or after delivery of anti-cancer immunotherapy. We have found that delivery of one (or more) of these SMV compounds will inhibit mitochondrial oxygen consumption within the tumor (both in host cells and tumor cells) and decrease tumor hypoxia in the cancerous tissue. The reduction in hypoxia in turn decreases the immunosuppressive effects of hypoxia and enhances naturally occurring, or therapeutically-stimulated, anti-cancer immune function.

Reduction of tumor hypoxia with SMV compounds enhances anti-cancer T cell effector function. The stimulation of effector T cell function is anticipated to be particularity helpful to, but not limited to, the effectiveness of immune-checkpoint blockade (ICB) immunotherapy. Other adaptive immune therapies are also likely to benefit from reduction in tumor hypoxia such as chimeric antigen receptor (CAR) T cell therapy, cancer vaccines, and adoptive T cell transfer methodologies. This sensitization is not expected to have adverse normal tissue toxicity as normal tissue rarely has pathological hypoxia.

The SMV compounds listed herein are not likely, on their own, to have extensive anti-cancer activity. Instead, these agents potentiate the efficacy of other anticancer agents such as adoptive cell transfer therapy (with endogenous T cells, CAR-T, TCR-T, NK cells, CAR-NK cells etc.), cytokine therapy (IL-2, IL-7, IL-21, IL-15, IL-12 and others), oncolytic agents (e.g., T-Vec), immune checkpoint blockade immunotherapy, including anti- PD-1 or anti-CTLA4 agents or other agents, either alone or in combination (with targeted therapies, conventional therapies, bispecific antibodies, antibody drug conjugates etc.), designed to block other regulators of immune function for the purpose of achieving an anti-cancer effect, such as, but not limited to, PDL2, CD28, CD80, CD86, 138RP1, B7-H4, VISTA (PD-IB), MEM, CDI, CD37L, OX4OL, CD70, CD40, GAL9, CD28, ICOS, BTLA, MR, LAGS, CD137, OX40, CD27, CD40L, TIM3, A2aR, CSF-1R, CSF-1, TEAT, IL-33, IL-18BP, IL-10 and others. Based on our disclosure and data we find it likely that SMV compounds may also be used to benefit instances other than cancer where enhanced immune function is desired.

PPVs PDE and mitochondrial activities are chemically separable. Papaverine has been traditionally thought to be effective as a vasodilator for the treatment of conditions including erectile dysfunction because it can inhibit PDE10A (like PDE 5 inhibitors sildenafil and tadalafil). However, PDE inhibition is not responsible for OCR effects in vitro, so we postulated that there are two activities in PPV, one inhibiting ETC complex 1 (C₁) and one inhibiting PDE10A that may reside in different parts of the molecule. We therefore defined the structure activity relationship (SAR) of PPV relative to two assays: inhibition of cellular OCR by Seahorse XE assay, and inhibition of PDE10A by purified enzyme activity (PDE10A activity assay, BPS Bioscience). Because PPV can compete with rotenone at complex 1 (23), we reasoned that they may be binding the same site and therefore the structures would be similar relative to their target. Because the top portion of PPV was a planar structure like that of rotenone, we decided to focus our attention on the lower portion of the molecule. Following this strategy, analysis of the first 41 derivatives showed that we have identified “relatively pure” C1 inhibitors (SMV32 and SMV37) and “relatively pure” PDE10A inhibitor (SMV23) (FIG. 1 and Table 1).

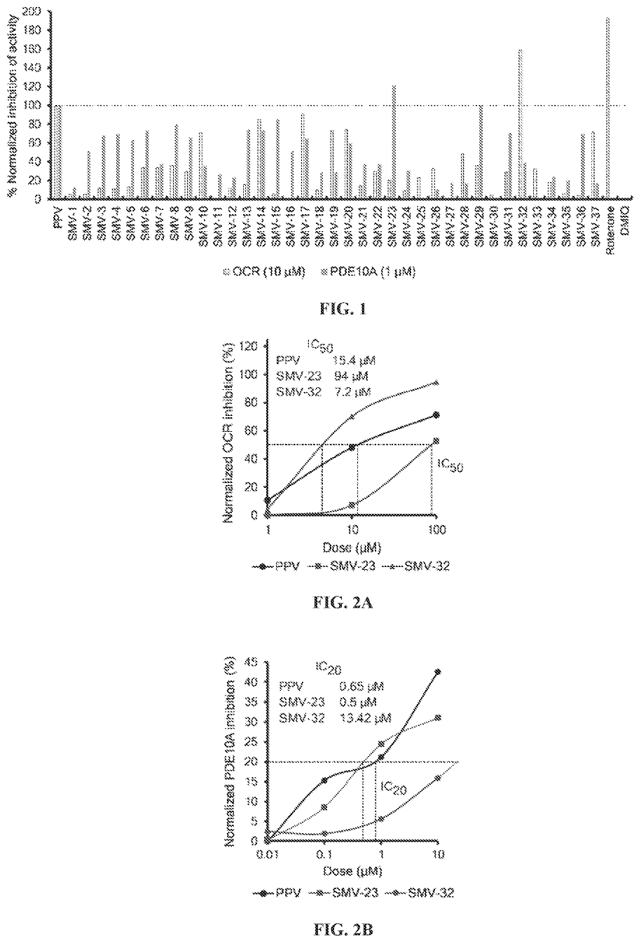

Careful dose response analysis shows that for OCR: PPV IC₅₀ is 15 μM, SMV23 94 μM, and SMV32 7.2 μM. When tested for PDE inhibition, PPV IC₂₅ is 0.65 μM, SMV23 0.5 μM and SMV32 13.4 μM, for over one log of separation in the two activities in these derivatives (FIG. 2). These lead compounds are synthesized from commercial precursors and purified on silica gel column. Structures were confirmed by ¹H NMR, found to be >95% pure by HPLC, and are soluble in aqueous solutions.

Papaverine and SMV32 enhance PD-1 checkpoint blockade immunotherapy for cancer treatment. Because of the literature describing the immunomodulatory effects of hypoxia⁽²⁴⁻²⁷⁾, we hypothesized that reducing hypoxia may also enhance tumor lymphocyte infiltration and function and improve immune-mediated tumor cell killing. As those of skill in the art recognize, there is a large body of literature and research on increase the supply side of the hypoxia equation, but it has been heretofore an unexpected approach to decrease the tri demand side of the hypoxia equation. We tested tumors grown from the relatively immune-sensitive syngeneic MC₃₈ colorectal tumor line in immuno-competent C₅₇₁₃₁₁₆ mice. We first determined that repeated dosing of either PPV or SMV32 alone had no effect on tumor growth. In a second cohort, we implanted 1×10⁶ cells/mouse and after 5 days, when tumors were 50-100 mm³, they were randomized into four groups. Therapy was started with either control rat IgG antibody, or a suboptimal dose of anti PD-1(CD279) blocking monoclonal antibody (BioXCell, clone 29F.1A12, 100 μg ip every 3 days). For the anti-PD-1 treated animals, two additional groups were treated 16 hours prior to antibody with 2 mg/kg PPV or SMV32 iv, FIG. 3 shows that addition of the hypoxia modifying agents significantly enhanced the effectiveness of the PD-1 blockade increasing the tumor growth delay (P<0.01 at days 8 and 11, variance large at day 13). Since PD-1 blockade is effective by enhancing the effector function of CD8⁺ cells, we interpret these results to indicate reducing hypoxia enhances cytotoxic anti-tumor lymphocyte activity by one or more mechanisms.

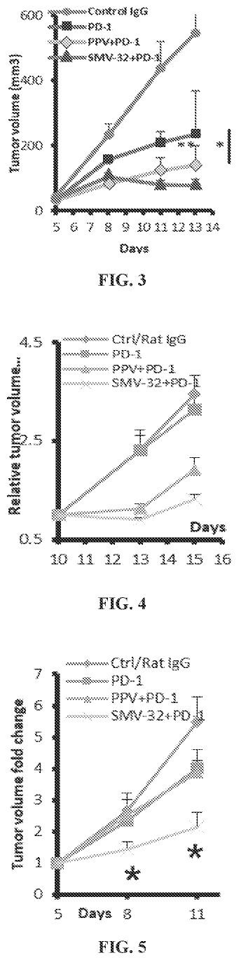

There are reports in the literature indicating ways that hypoxia affects immune functions of lymphocytes, MDSCs and T regulatory cells infiltrating in the tumor. While there may be ways to overcome each of these effects individually, it is more efficient to reverse them all simultaneously by reversing hypoxia. We first found that treatment of mice with PPV and SMV can enhance activity of PD-1 blockade in the sensitive MC₃₈ model in FIG. 2, so we next tested our therapy in the intermediately sensitive CMT167 lung cancer model. In heterotopic tumors grown in the flank, we find that SMV32 is superior to PPV at immune-sensitizing tumors when given 16 hours prior to a sub-optimal dose of anti-PD-1 antibody (FIG. 4). We see no activity from this dose of PD-1 blocking antibody alone, but significant tumor growth delay in the groups treated with either PPV or SMV32 prior to anti-PD1 antibody. SMV32 appears to be superior to PPV in the level of tumor growth delay when given at the same dose as PPV. We hypothesize that this is due to enhanced pharmacokinetics and enhanced duration of response that we have detected in in vitro washout experiments.

Finally, as a rigorous test of anti-hypoxia agents as immuno-sensitizers of model tumors grown in mice, we next tested the PD-1 blockade resistant tumor model Lewis Lung Carcinoma (LLC). These tumors have been reported to be refractory to even full dose (200 ug/mouse) PD-1 blocking antibody. FIG. 5 shows that in our experiment that these tumors are in fact resistant to treatment by PD-1 blocking antibody alone, and even treatment with papaverine first and PD-1 antibody second shows no therapeutic benefit. However, treatment of tumor bearing animals with SMV32 first and PD-1 antibody second showed significant growth inhibition. These results support the model that SMV32 is superior to PPV as an immuno-sensitizer for treatment with PD-1 immune checkpoint blockade therapy for highly resistant tumor models.

Taken together, these results indicate that treatment with anti-hypoxia PPV derivatives have no significant effect on their own, but effectively enhance sensitivity of tumors to immunotherapy by immune checkpoint blockade by PD-1 blocking antibody. However, these results indicate that novel PPV derivatives could also sensitize tumors to other anti-cancer immunotherapies where hypoxia is a limiting factor such as anti-CTLA4 antibody, adoptive immune cell transfer, oncolytic virotherapy, or chimeric antigen receptor (CAR) T cell therapy. In order to determine a mechanism by which reduction in hypoxia enhances immune function in the solid tumor, we undertook an investigation with multi spectral immunophenotyping. This technology uses 36 different markers simultaneously to characterize immune cell infiltrate in tumors. We performed this analysis on heterotopic NIC₃₈ murine colorectal carcinoma tumors grown in immune-competent C₅₇BI6 mouse strain. A cohort of tumor bearing mice were randomized to be treated with either IV delivery of vehicle control, or either PPV or SMV32. Animals were treated with 2 mg/kg of vehicle control or PPV or SMV32 on days 8, 10, and13 after tumor inoculation and tumors were harvested at day 15, We used unbiased cluster analysis to determine how reversing hypoxia shifts the lymphocyte population and function in the tumor using the following markers: Viability, CD45, CD3, CD8, CD4, PoxP3, CD11b, PD-1, Tim3, Slamf6, TOX, Tcf-1, CD44, CD62L, CTAL4, Lag-3, Kirg1, T-bet, Ki-67, Bcl2, FOMES, Vista, TIGIT, CX3CR1, ICOS, CD27, CD38, CD95, NK1.1, CD25, GITR, CD69, GZMB, pimonidazole.

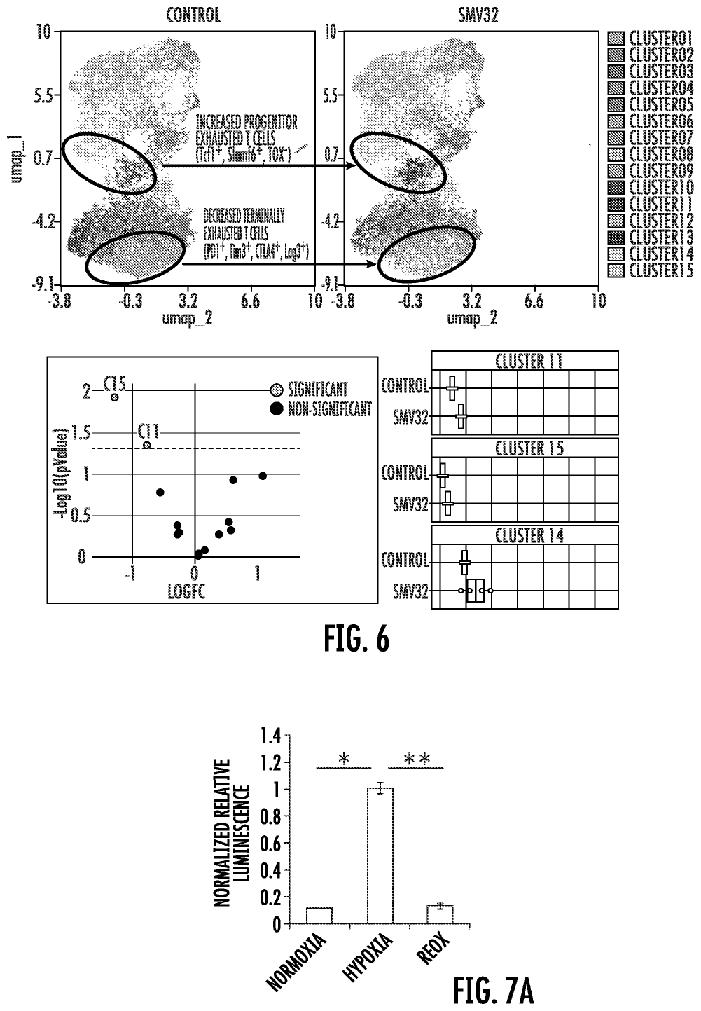

Analysis of immune cells in MC38 tumor bearing animals in this experiment revealed no changes in spleen or lymph node populations, but significant changes in tumor infiltrating lymphocytes (FIG. 6). Multispectral flow cytometry with a cocktail of 24 antibodies and unbiased statistical analysis using OMIQ, an online program using flowSOM clustering and Lit AP dimension reduction, found statistically significant changes (edge R and significance analysis of microarrays) in two groups of cytotoxic CDS lymphocytes. After SMV32 treatment, the progenitor exhausted (Tcf1^{High}, Slamf6^{High}, TOX^{Low}) and terminally exhausted (PD1^{High}, Tim3^{High}, Ctla4^{High}, Lag3^{High}), populations showed a shift from terminal (known to be non-functional) to progenitor cells (known to be able to convert to rejuvenated T cells after PD-1 blockade). This shift will likely increase the fraction of lymphocytes in the tumor that will respond to PD-1 blockade with increased antitumor function⁽⁹⁹⁾, consistent with functional ire results in FIG. 3.

TABLE 1
papaverine derivatives structure and activity.

| | | Inhibitory activity | |
|---|---|---|---|
| | | OCR | |
| | MW | % of | PDE10A |
| Compound No. | (g/mol) | PPV | % of PPV |
| 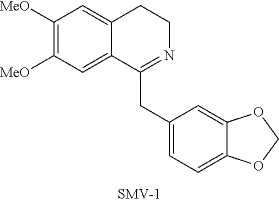 [SMILES: COc1cc2c(cc1OC)C(Cc1ccc3c(c1)OCO3)=NCC2] | 325.36 | 5.55 | 12.77 |
| 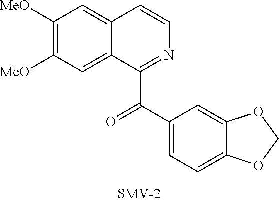 [SMILES: COc1cc2ccnc(C(=O)c3ccc4c(c3)OCO4)c2cc1OC] | 337.33 | 5.55 | 51.12 |
| 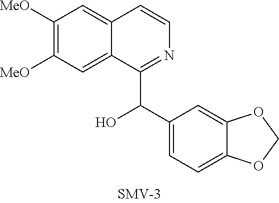 [SMILES: COc1cc2ccnc(C(O)c3ccc4c(c3)OCO4)c2cc1OC] | 339.35 | 12.96 | 67.90 |
| 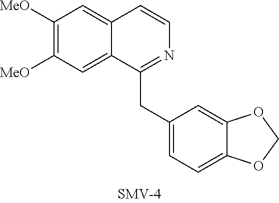 [SMILES: COc1cc2ccnc(Cc3ccc4c(c3)OCO4)c2cc1OC] | 323.35 | 11.11 | 69.54 |
| 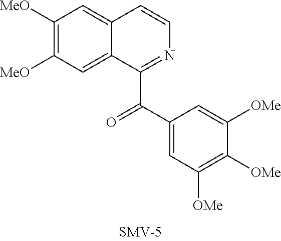 [SMILES: COc1cc2ccnc(C(=O)c3cc(OC)c(OC)c(OC)c3)c2cc1OC] | 383.40 | 13.63 | 63.39 |
| 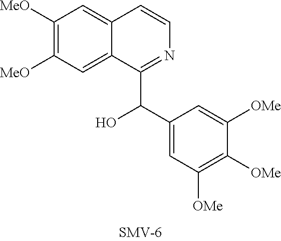 [SMILES: COc1cc2ccnc(C(O)c3cc(OC)c(OC)c(OC)c3)c2cc1OC] | 385.42 | 34.09 | 72.86 |
| 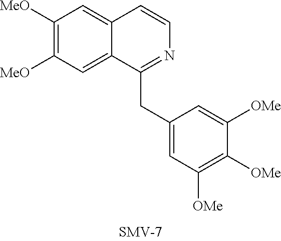 [SMILES: COc1cc2ccnc(Cc3cc(OC)c(OC)c(OC)c3)c2cc1OC] | 369.42 | 34.09 | 37.37 |
| 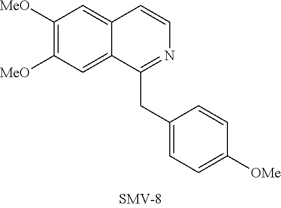 [SMILES: COc1ccc(Cc2nccc3cc(OC)c(OC)cc23)cc1] | 309.37 | 36.36 | 79.93 |
| 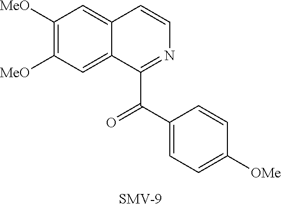 [SMILES: COc1ccc(C(=O)c2nccc3cc(OC)c(OC)cc23)cc1] | 323.35 | 29.55 | 65.88 |
| 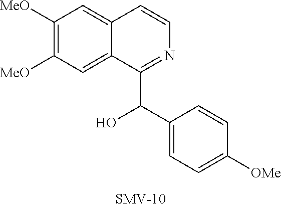 [SMILES: COc1ccc(C(O)c2nccc3cc(OC)c(OC)cc23)cc1] | 325.36 | 70.45 | 35.44 |
| 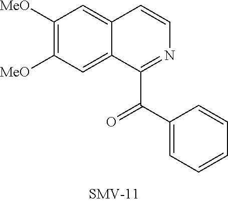 [SMILES: COc1cc2ccnc(C(=O)c3ccccc3)c2cc1OC] | 293.32 | 0 | 26.28 |
| 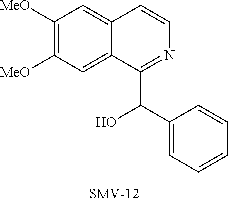 [SMILES: COc1cc2ccnc(C(O)c3ccccc3)c2cc1OC] | 295.34 | 12.07 | 23.60 |
| 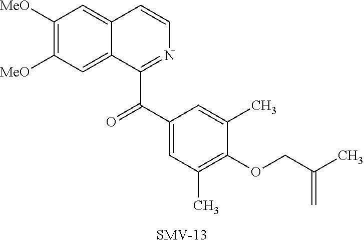 [SMILES: C=C(C)COc1c(C)cc(C(=O)c2nccc3cc(OC)c(OC)cc23)cc1C] | 391.47 | 15.51 | 74.38 |
| 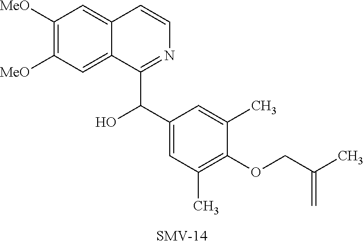 [SMILES: C=C(C)COc1c(C)cc(C(O)c2nccc3cc(OC)c(OC)cc23)cc1C] | 393.48 | 84.42 | 73.33 |
| 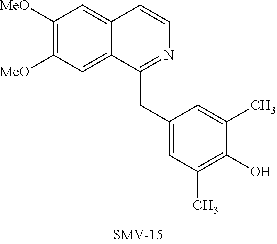 [SMILES: COc1cc2ccnc(Cc3cc(C)c(O)c(C)c3)c2cc1OC] | 323.39 | 5.86 | 85.02 |
| 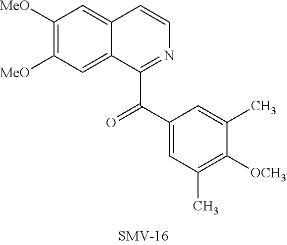 [SMILES: COc1cc2ccnc(C(=O)c3cc(C)c(OC)c(C)c3)c2cc1OC] | 351.40 | 0 | 50.92 |
| 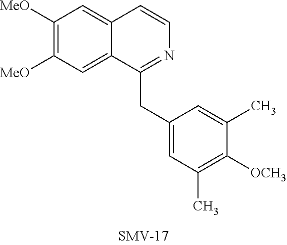 [SMILES: COc1cc2ccnc(Cc3cc(C)c(OC)c(C)c3)c2cc1OC] | 337.42 | 91.18 | 64.65 |
| 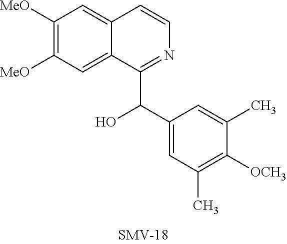 [SMILES: COc1cc2ccnc(C(O)c3cc(C)c(OC)c(C)c3)c2cc1OC] | 353.42 | 10.35 | 29.18 |
| 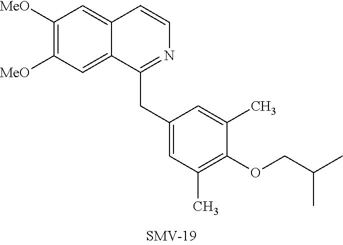 [SMILES: COc1cc2ccnc(Cc3cc(C)c(OCC(C)C)c(C)c3)c2cc1OC] | 379.50 | 72.88 | 29.13 |
| 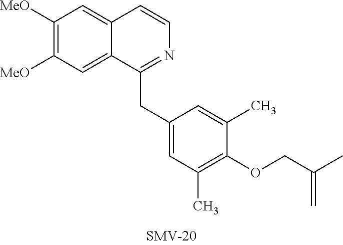 [SMILES: C=C(C)COc1c(C)cc(Cc2nccc3cc(OC)c(OC)cc23)cc1C] | 377.48 | 74.81 | 59.69 |
| 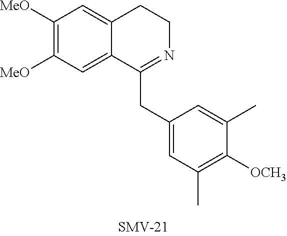 [SMILES: COc1cc2c(cc1OC)C(Cc1cc(C)c(OC)c(C)c1)=NCC2] | 339.44 | 14.81 | 37.27 |
| 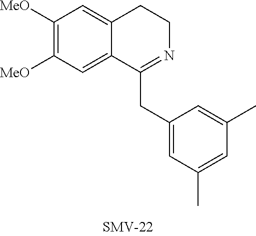 [SMILES: COc1cc2c(cc1OC)C(Cc1cc(C)cc(C)c1)=NCC2] | 309.41 | 30.09 | 40.00 |
| 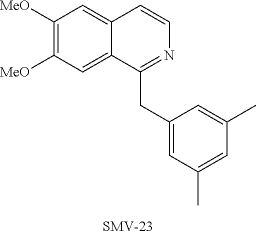 [SMILES: COc1cc2ccnc(Cc3cc(C)cc(C)c3)c2cc1OC] | 307.39 | 20.37 | 121.49 |
| 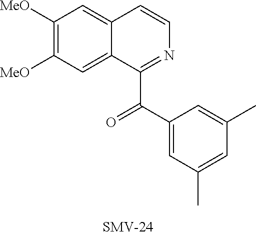 [SMILES: COc1cc2ccnc(C(=O)c3cc(C)cc(C)c3)c2cc1OC] | 321.38 | 9.26 | 30.52 |
| 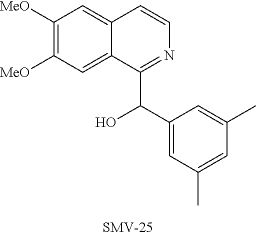 [SMILES: COc1cc2ccnc(C(O)c3cc(C)cc(C)c3)c2cc1OC] | 323.39 | 23.40 | 0 |
| 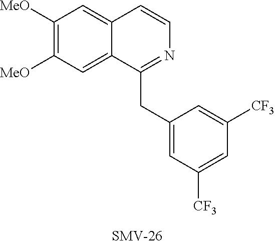 [SMILES: COc1cc2ccnc(Cc3cc(C(F)(F)F)cc(C(F)(F)F)c3)c2cc1OC] | 415.34 | 33.4 | 10.28 |
| 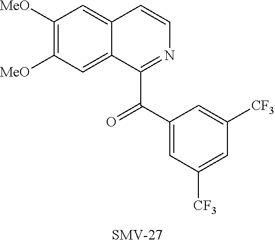 [SMILES: COc1cc2ccnc(C(=O)c3cc(C(F)(F)F)cc(C(F)(F)F)c3)c2cc1OC] | 429.32 | 0 | 17.69 |
| 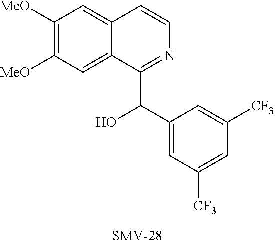 [SMILES: COc1cc2ccnc(C(O)c3cc(C(F)(F)F)cc(C(F)(F)F)c3)c2cc1OC] | 431.33 | 48.27 | 16.58 |
| 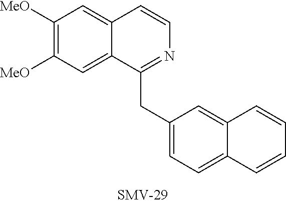 [SMILES: COc1cc2ccnc(Cc3ccc4ccccc4c3)c2cc1OC] | 329.40 | 35.99 | 100 |
| 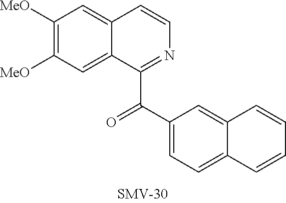 [SMILES: COc1cc2ccnc(C(=O)c3ccc4ccccc4c3)c2cc1OC] | 343.38 | 4.34 | 0 |
| 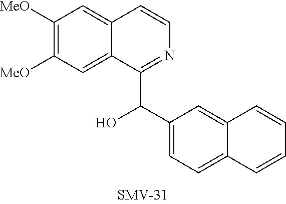 [SMILES: COc1cc2ccnc(C(O)c3ccc4ccccc4c3)c2cc1OC] | 345.40 | 29.26 | 70.84 |
| 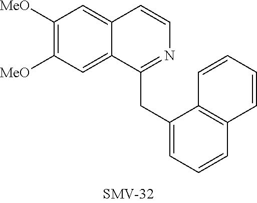 [SMILES: COc1cc2ccnc(Cc3cccc4ccccc34)c2cc1OC] | 329.40 | 159.3 | 38.66 |
| 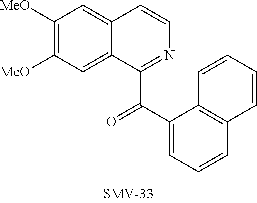 [SMILES: COc1cc2ccnc(C(=O)c3cccc4ccccc34)c2cc1OC] | 343.38 | 31.91 | 0 |
| 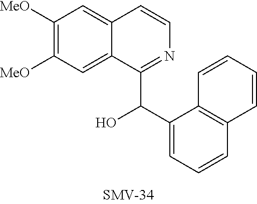 [SMILES: COc1cc2ccnc(C(O)c3cccc4ccccc34)c2cc1OC] | 345.40 | 18 | 24.24 |
| 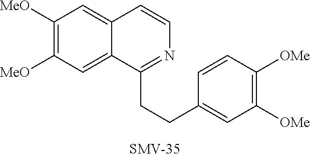 [SMILES: COc1ccc(CCc2nccc3cc(OC)c(OC)cc23)cc1OC] | 353.42 | 5.94 | 20.16 |
| 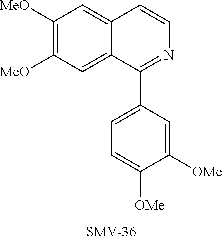 [SMILES: COc1ccc(-c2nccc3cc(OC)c(OC)cc23)cc1OC] | 325.36 | 3.64 | 69.55 |
| 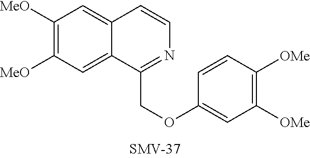 [SMILES: COc1ccc(OCc2nccc3cc(OC)c(OC)cc23)cc1OC] | 355.39 | 72.05 | 17.18 |
| 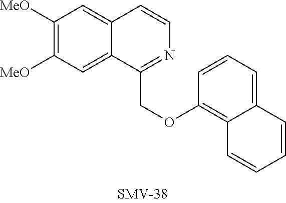 [SMILES: COc1cc2ccnc(COc3cccc4ccccc34)c2cc1OC] | 345.40 | 14.19 | N/A |
| 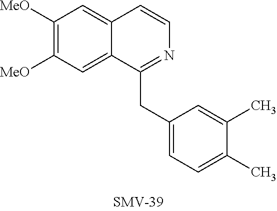 [SMILES: COc1cc2ccnc(Cc3ccc(C)c(C)c3)c2cc1OC] | 307.49 | 17.43 | N/A |
| 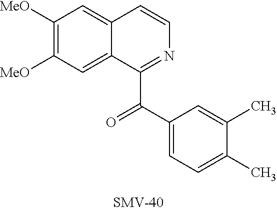 [SMILES: COc1cc2ccnc(C(=O)c3ccc(C)c(C)c3)c2cc1OC] | 321.38 | 9.64 | N/A |
| 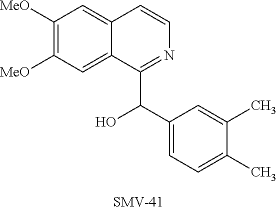 [SMILES: COc1cc2ccnc(C(O)c3ccc(C)c(C)c3)c2cc1OC] | 323.39 | 22.69 | N/A |
| 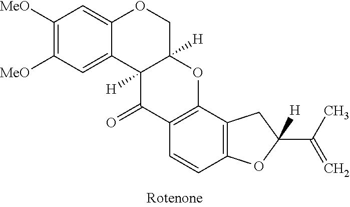 [SMILES: C=C(C)[C@H]1Cc2c(ccc3c2O[C@@H]2COc4cc(OC)c(OC)cc4[C@@H]2C3=O)O1] | 394.41 | 193 | 0 |
| 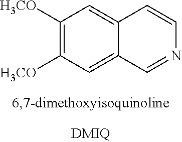 [SMILES: COc1cc2ccncc2cc1OC] | 189.21 | 0 | 0 |

### LITERATURE CITED

1. De Takats G. Papaverine: use and toxicity. N Engl J Med. 1970; 282(4225. doi: PubMed PMID: 5409818.
2. Merk G. “Vorlaufige Notiz über eine neue organische Base inn Opium” [Preliminary notice of a new organic base in opium]. Annalen der Chemie Lind Pharmacie. 1848; 66:125-8. doi: 10.1002/jlac.18480660121.
3. Dewhirst M W, Vinuya R Z, Ong E T, Klitzman B, Rosner G, Secomb T W, Gross N. Effects of bradykinin on the hemodynamics of tumor and granulating normal tissue microvasculature. Radiat Res. 1992,130(3):345-54. PubMed PMID: 1594761.
4. Shan S Q, Rosner G L, Braun R D, Hahn J, Pearce C, Dewhirst M W. Effects of diethylamine/nitric oxide on blood perfusion and oxygenation in the R3230Ac mammary carcinoma. Br J Cancer. 1997; 76(4):429-37. PubMed PMID: 9275018; PMCID: 2227992.
5. Pinter M, Jain R K. Targeting the renin-angiotensin system to improve cancer treatment: Implications for immunotherapy. Sci Transl Med. 2017; 9(410). doi: PubMed PMID: 28978752; PMCID: PMC_{5928511.}
6. Liu H, Naxerova K, Pinter M, Incio J, Lee H, Shigeta K, Ho W W, Crain J A, Jacobson A, Michelakos T, Dias-Santos D, Zanconato A, Hong T S, Clark J W, Murphy J E, Ryan D P, Deshpande V, Lillemoe K D, Fernandez-Del Castillo C, Downes M, Evans R M, Michaelson J, Ferrone C R, Boucher Y, Jain R K. Use of Angiotensin System Inhibitors Is Associated with Immune Activation and Longer Survival in Nonmetastatic Pancreatic Ductal Adenocarcinoma. Clin Cancer Res. 2017; 23(19):5959-69. doi: 10.1158/1078-0432.CCR-17-0256. PubMed PMID: 28600474; PMCID: PMC_{5856249.}
7. Benej M, Hong X, Vibhute S, Scott S, Wu J, Graves E, Le Q T, Koong A C, Giaccia A J, Yu B Chen S C, Papandreou I, Denko N C. Papaverine and its derivatives radiosensitize solid tumors by inhibiting mitochondrial metabolism. Proc Nati Acad Sci U S A. 2018; 115(42):10756-61, doi: 10.1073/pnas.1808945115. PubMed PMID: 30201710; PMCID: PMC_{6196495.}
8. Hockel M, Schlenger K, Mitze M, Schaffer U, Vaupel P. Hypoxia and Radiation Response in Human Tumors, Semin Radiat Oncol. 1996; 6(1):3-9. doi: PubMed. 10717157.
9. Nordsmark M. Overgaard J. A confirmatory prognostic study on oxygenation status and loco-regional control in advanced head and neck squamous cell carcinoma treated by radiation therapy. Radiother Oncol, 2000; 57(1)39-43. PubMed PMID: 11033187.
10. Brizel D M, Scully S P, Harrelson J M, Layfield L J, Bean J M, Prosnitz L R, Dewhirst MW. Tumor oxygenation predicts for the likelihood of distant metastases in human soft tissue sarcoma. Cancer Res. 1996; 56(5):941-3. PubMed PMID: 8640781.
11. Stadler P, Becker A. Feldmann H J, Hansgen G, Dunst J, Wurschmidt F, Molls M. Influence of the hypoxic subvolume on the survival of patients with head and neck cancer. Int J Radiat Oncol Biol Phys. 1999; 44(4):749-54. PubMed PMID: 10386631.
12. Fyles A W, Milosevic M, Wong R, Kavanagh M C, Pintilie M, Sun A, Chapman W, Levin W, Manchul L, Keane T J, Hill RP. Oxygenation predicts radiation response and survival in patients with cervix cancer. Radiother Oncol, 1998; 48(2):149-56. PubMed PMID: 9783886.
13. Vanselow B, Eble Rudat V, Wollensack P, Conradt C, Dietz A. Oxygenation of advanced head and neck cancer: prognostic marker for the response to primary radiochemotherapy. Otolaryngol Head Neck Surg. 2000; 122(6):856-62. doi:10.1016/S0194-59980070014-3, PubMed PMID: 10828799.
14. Knocke T H, Weitmann H D, Feldmann H J, Selzer E, Potter R. Intratumoral pO2-measurements as predictive assay in the treatment of carcinoma of the uterine cervix. Radiother Oncol. 1999; 53(2):99-104. PubMed PMID: 10665785.
15. Griffin R J, Okajima K, Song C W. The optimal combination of hyperthermia and carbogen breathing to increase tumor oxygenation and radiation response. Int J Radiat Oncol its Biol Phys. 1998; 42(4865-9. PubMed PMID: 9845112.
16. Siemann D W, Kochanski K. Combinations of radiation and misonidazole in a murine lung tumor model. Radiat Res. 1981; 86(4387-97. PubMed PMID: 7232698.
17. Brown J M, Lemmon M J. Tumor hypoxia can be exploited to preferentially sensitize tumors to fractionated irradiation. Int J Radiat Oncol Biol Phys. 1991; 20(4457-61. PubMed PMID: 1995531.
18. Mendenhall W M, Morris C G, Amdur R J, Mendenhall N P, Siemann D W. Radiotherapy alone or combined with carbogen breathing for squamous cell carcinoma of the head and neck: a prospective, randomized trial. Cancer. 2005; 104(2):332-7, doi: 10.1002/cncr.21146. PubMed PMID: 15937906.
19. Busk M, Elorsman M R. Relevance of hypoxia in radiation oncology: pathophysiology, tumor biology and implications for treatment. Q J Nucl Med. Mol Imaging. 2013; 57(3):219-34. PubMed PMID: 24045619.
20. Rischin D, Peters L J, O'Sullivan B, Giralt J, Fisher R, Yuen K, Trotti A, Bernier Bourhis J, Ringash J Henke M, Kenny L. Tirapazamine, cisplatin, and radiation versus cisplatin and radiation for advanced squamous cell carcinoma of the head and neck (TROG 02.02, fIeadSTART): a phase III trial of the Trans-Tasman Radiation Oncology Group, J Clin Oncol. 2010; 28(18):2989-95. doi: 10.1200/JCO.2009.27.4449. PubMed PMID: 20479425.
21. Lee D J, Cosmatos D. Marcial V A, Fit K K, Rotman M, Cooper J S, Ortiz H G, Beitler J J, Abrams R A, Curran W J, et al. Results of an RTOG phase III trial (RTOG 85-27) comparing radiotherapy plus etanidazole with radiotherapy alone for locally advanced head and neck carcinomas. Int J Radiat Oncol Biol Phys. 1995; 34:9:567-76, doi: 10.1016/0360-3016(95)00150-W. PubMed PMID: 7790241.
22. Vaupel P. Tumor microenvironmental physiology and its implications for radiation oncology. Semin Radiat Oncol. 2004; 14(3):198-206. doi: 10.1016/j.semradonc.2004.04.008. PubMed PMID: 15254862.
23. Benej M, Hong X, Vibhute S. Scott S. J, Graves F, Le Q T, Koong A C, Giaccia A J, Yu B, Chen C S, Papandreou I, Denko N C. Papaverine and its derivatives radiosensitize solid tumors by inhibiting mitochondrial metabolism. Proceedings of the National Academy of Sciences of the United States of America. 2018; 115(42):10756-61. doi: 10.1073/pnas.1808945115. PubMed PMID: 30201710; PMCID: PMC_{6196495.}
24. Noman M Z, Janji B, Abdou A, Hasmim M, Terry S, Tan T Z, Mami-Chouaib F, Thiery J P, Chouaib S. The immune checkpoint ligand PD-L1 is upregulated in EMT-activated human breast cancer cells by a mechanism involving ZEB-i and miR-200. Oncoimmunology. 2017; 6(1):e1263412. doi: 10.1080/2162402X.2016.1263412. PubMed PMID: 28197390; PMCID: 5283623.
25. Jayaprakash P, Ai M, Liu A, Budhani P, Bartkowiak T, Sheng J, Ager C, Nicholas C, Jaiswal A R, Sun Y, Shah K, Balasubramanyam S, Li N, Wang G, Ning J. Zal A, Zal T, Curran M A. Targeted hypoxia reduction restores T cell infiltration and sensitizes prostate cancer to immunotherapy. J Clin Invest. 2018; 128(11):5137-49. doi.: 10,11.72/J096268. PubMed PMID: 30188869; PMCID: PMC6205399.
26. Dewhirst M W, Mowery Y M, Mitchell J B, Cherukuri M K, Secomb T W. Rationale for hypoxia assessment and amelioration for precision therapy and immunotherapy studies. J Clin Invest. 2019; 129(2):489-91. doi: 10.1172/JCI126044. PubMed. PMID: 30614815; PMCID: 6355230.
27. Riera-Domingo C, Audige A, Granja S, Cheng WC, Ho PC, Baltazar F, Stockinann C, Mazzone M. Immunity, Hypoxia, and Metabolism-The Menage a Trois of Cancer: Implications for Immunotherapy. Physiological reviews. 2020; 100(1):1-102. doi:10.1152/physrev.00018.2019. PubMed PMED: 31414610.

### Example 2

### Pharmacological Regulation of Tumor Hypoxia in Model Murine Tumors and Spontaneous Canine Tumors

Simple Summary: Tumor hypoxia is a state of low oxygen tension typically occurring in most solid tumors because the oxygen supply does not meet the metabolic demand of the tissue. Hypoxia has been associated with increased resistance to anti-cancer therapy for decades. Reducing oxygen demand with therapeutic targeting of mitochondrial oxidative metabolism can mitigate tumor hypoxia. We show that pharmacological regulation of mitochondrial metabolism has a direct impact on the levels of tumor hypoxia in murine tumor models and spontaneous canine soft tissue sarcomas.

Background: Hypoxia is found in many solid tumors and is associated with increased disease aggressiveness and resistance to therapy. Reducing oxygen demand by targeting mitochondrial oxidative metabolism is an emerging concept in translational cancer research aimed to reduce hypoxia. We have shown that the FDA approved drug papaverine and its novel derivative SMV-32 are potent mitochondrial complex 1 inhibitors.

Methods: We use a dynamic in vivo luciferase reporter system, pODD-Luc, to evaluate the impact of pharmacological manipulation of mitochondrial metabolism on the levels of tumor hypoxia in transplanted mouse tumors. We also imaged canine patients with blood oxygen level determination (BOLD) MRI at baseline and one hour after a dose of 1 or 2 mg/kg papaverine.

Results: We show that pharmacological suppression of mitochondrial oxygen consumption (OCR) in tumor bearing mice increases tumor oxygenation while stimulation of mitochondrial OCR decreases tumor oxygenation. In parallel experiments in a small series of spontaneous canine sarcomas treated at OSLO Veterinary Medical Center, we observed a significant increase in BOLD signal indicative of an increase in tumor oxygenation of up to 10-50 mm Hg O₂.

Conclusion: In both transplanted murine tumors and spontaneous canine tumors we found that decreasing mitochondrial metabolism can decrease tumor hypoxia, potentially offering a therapeutic advantage.

Introduction

Tumor hypoxia is a highly heterogenous and a function of local oxygen supply and demand within the solid tumor. Hypoxia typically arises by one of two mechanisms. First is diffusion limited (or chronic) hypoxia where oxygen diffuses from a blood vessel and is consumed as it passes through cells dropping in amount the farther it goes, typically reaching stressful levels at 100-150 μm, originally suggested in [1]. Second is perfusion limited (or acute) hypoxia and is caused by inhomogeneity in red blood cell flux through chaotic tumor blood vessels [2,3]. Cellular adaptive response to hypoxia is orchestrated by multiple mechanisms with transcription factor HIF-1 playing an important role [4]. The HIF1α subunit is regulated at the protein level by oxygen tension [5]. In well oxygenated conditions HIF1α is hydroxylated by prolyl hydroxylases on proline resides 402 and 564. These modified residues are recognized by the E3 ubiquitin ligase pVHL and HIF1α is marked with polyubiquitin for proteasomal degradation [6]. Because the prolyl hydroxylases use molecular oxygen as a substrate, when oxygen is low, HIF1α is not modified and the protein becomes stabilized [7]. Several downstream HIFI target genes function to bring the oxygen supply and demand back in balance in part by suppressing mitochondrial oxidative metabolism and upregulating the glycolytic flux [8, 9]. These complex changes in cancer cell biology promote tumor heterogeneity and are associated with poor clinical prognosis, more aggressive tumor phenotype and increased resistance to anti-cancer therapy [10-13].

Pharmacological manipulation of oxygen demand is an emerging concept in the decades-long (and so far unfruitful) quest to reduce tumor hypoxia and sensitize tumors to anti-cancer therapy. Mitochondrial oxygen consumption (OCR) constitutes approximately 90% of cellular oxygen demand in oxidative phosphorylation (OXPHOS). This process couples carbon source oxidation with ATP generation in a series of redox reactions transferring electrons from reduced cofactors to molecular oxygen [14]. The electrons are passed through four mitochondii al electron transport chain complexes (I-IV) to oxygen while generating an electrochemical gradient that fuels ATP generation via F0F1 ATP synthase [15]. Mechanistically, a decrease in mitochondrial oxygen consumption would increase the distance of oxygen diffusion within the tumor, allowing chronically hypoxic areas to become re-oxygenated. This principle has been explored in several preclinical strategies supporting the model that mitochondria(OCR has a causal relationship to the levels of tumor hypoxia [16-20].

In this study, we present data showing that biochemical manipulation of mitochondrial OCR modulates the expression of the ODI)-luc transgene in tumors grown in mice. In vitro, cells expressing ODD-luc showed increased luciferase signal when cultured in hypoxia or with hypoxia mimetics. We also show that mice bearing tumors with ODD-luc expression had significantly lower level of hypoxic luciferase activity when breathing carbogen (95% O₂, 5% CO₂) instead of room air. We further found that strategies that either stabilize HIFla or stimulate mitochondrial OCR increase ODD-luc signal while suppression of OCR decreases ODD-laic; signal. We have previously shown that papaverine, an FDA-approved vasorela.xa.nt with mitochondrial complex 1 inhibitor activity effectively reduces hypoxic tumor fractions in vivo [19]. Here we show that its novel papaverine derivative SMV-32 that acts as complex I inhibitor with reduced PDE10A effect mediates superior OCR inhibition in both orthotopic and heterotopic mouse tumor models. We extended these murine findings in an initial cohort of spontaneous canine soft tissue sarcoma (STS). Patients being treated for spontaneously arising STS at the OSU Veterinary Medical Center were recruited to participate in an NCI funded test of papaverine as an effective anti-hypoxia agent. We determined changes in blood oxygen level determination (BOLD) _MRI imaging parameters in these tumors after a single dose of 1 or 2 mg/kg papaverine IV. BOLD MRI detects the paramagnetic differences in oxy versus deoxyhemoglobin [22]. Initial analysis indicated a fraction of tumors showed significant increase in tumor oxygenation. These findings further establish the direct impact of OCR manipulation on biologically significant tumor hypoxia and support the model that mitochondrial metabolism can drive tumor oxygenation in a potentially therapeutic setting.

Materials and Methods

Construction, tumor growth, and visualization of pODDLuciferase expressing cells. HeLa (cervical cancer, MUD: CVCL_0030) and MiaPaca-2 (MP2) (pancreatic cancer, RRID: CVCL_0428) cancer cell lines were obtained from American Type Culture Collection (ATCC). Stable HeLa pODD and MP2 pODD cell lines were generated by direct transfection with pLenti-pODD-Pura vector generated by subcloning the ODD-Luc cassette from ODD-Luciferase-pcDNA3 vector (Addgene #18965) into bicistronic pLenti-C-Myc-DDKIRES-Puro Lentiviral Gene Expression Vector (Origene #PS100069). Briefly, pODD Luc cassette was amplified using following primers adding AscI and PmeI restriction sites:

ODD-luciferase-Ascl-F ATATAggcgcgccgaattcgccaccatggaatt (SEQ ID NO: 1)

ODD-luciferase-PmeI-R ATATAgMaaacattacacggcgatctttccg (SEQ ID NO: 2)

PCR-amplified cassette was then digested with Asci and Pmel restriction enzymes and ligated into pLenti-C-Myc-DDK-IRES-Puro. Correct orientation and sequence was confirmed by Sanger DNA sequencing. HeLa pODD and MIP2 pODD transfectants were selected with 2 μg/ml Puromycin for two weeks. Stable MP2 CMV Cells were generated by lentiviral transduction of MP2 cells with pLenti-CMV-Puro-Luc lentiviral vector (Addgene#17477) with 2 Puromycin selection for two weeks. Cells were grown in Dulbecco's Modified Eagle's Medium (DMEM) containing 25 mM D-glucose, 4 mM glutamine and 44 mM sodium bicarbonate in 5% CO₂, if not stated otherwise. The cells were exposed to hypoxia (1% O₂) in H35 Hypoxic Workstation (Hypoxygen) with 5% CO₂ at 37° C. In some experiments, we used a hypoxia mimetic dimethyloxalyglycine (DMOG) as described. Cell counts and cellular proliferation were established using hemocytometer to determine the cell number and trypan blue exclusion assay to establish the fraction of viable/dead cells. All human cell lines have been authenticated using STR profiling within the past 3 years. All experiments were performed with mycoplasma-free cells.

Orthotopic Panreatic Xenografts. MP2 PD AC cells (1×10⁶) or the indicated derivates were implanted in immune-deficient athymic nu/nu mice following fACLIC approved protocols. Briefly, an incision was placed in the right flank of anesthetized animals the spleen and pancreas liberated. Cells were mixed 1:1 with Matfigel and implanted in the tail of the pancreas in 10 μl. Incision was sutured in 2 layers, animals given long lasting analgesics, and allowed to recover from anesthesia. Mice were returned to housing for post-op care and monitored daily for 4 days. Animals were checked weekly after that by palpation to determine tumor growth.

Western Blotting Proteins were extracted with 1% v/v Triton X-100, 0.5% w/v Nonidet its NP-40, 150 mM NaCl, 50 mM Tris, pH 7.5, quantified by BCA Kit (Thermo Scientific) and. separated on 10% SDS-PAGE under reducing conditions. Proteins of interest were detected using antibodies against firefly luciferase (Abcam) and β-actin (Santa Cruz Biotech). All replicates of the individual experiments were analyzed from the respective run with appropriate loading controls.

In vitro luciferase Assay. Proteins were extracted using Luciferase Assay Lysis Buffer (125 mM Tris-HCl, pH 7.5, 11.0 mM DTT, 10 mM EGTA, 50% Glycerol, 5% Triton X-100) and 200 μl/sample were transferred to 96-well plate and D-luciferin was added to the final concentration of 150 μg/ml immediately before the assay. Bioluminescence was measured on a Synergy H1 Hybrid Plate Reader (Biotek) after a 10-minute incubation period (10 slsample, Gain=150) in triplicates.

In vivo bioluminescent imaging. All animal experiments were performed according to protocols approved by the OSU institutional IAGLIC review (protocol #201200000124-R2), with daily veterinarian observation. For heterotopic flank tumor experiments, 1×10⁶ HeLa pODD or 5×10⁶ MP2 cells were injected subcutaneously (s.c.) into the flanks of 6-wk-old female (HeLa pODD) or male (N1P2 pODD or MP2 CMV) immunocompromised athymic nu/nu mice. Replicate experiments with groups of 4-6 mice were used for statistical reproducibility. Caliper measurements of opposing diameters were used to calculate the tumor volumes using the modified ellipsoidal formula: tumor volume=(length×width²)[23]. Upon reaching 100 mm³, the animals were given 100 mg/kg D-luciferin intraperitoneally and bioluminescence images were captured using IVIS In Vivo Imaging System Lumina II (Perkin Elmer Inc). Mice were anesthetized with 1.5% isoflurane and bioluminescence was measured every two minutes for the period of 25 minutes to identify peak response of each mouse to D-luciferin. Bioluminescent data were analyzed using living Image software (Perkin Elmer Inc) and calculated as photon flux/sec. Obtained individual photon flux peak values were then normalized to baseline for each mouse and averaged per group. Gas inhalation experiments were performed by switching medical air and carbogen gas sources for the IVIS anesthesia vaporizer. Initial baselines were established in medical air under anesthesia, then the mice were subjected to 30 minutes of either medical air or carbogen rebreathing in an air-tight chamber while awake. After 30 minutes, photon flux was established in respective gas sources as a carrier for isoflurane anesthesia.

BOLD MRI analysis of canine tumors. Canine patients with primary sarcomas were enrolled in a trial to evaluate the effects of papaverine delivered at a dose of 1 or 2 mg/kg using ire blood oxygen level dependent (BOLD) magnetic resonance imaging (MRI). Dogs were anesthetized with isoflurane in medical air to effect on the bed of a Philips ingenia 3.071 UR scanner. They were first imaged with T1- (TR: 700ms, TE: 9ms, resolution 0.3×0.3×3 mm) and T2-weighted (TR: 3000 ms, TE: 80ms, resolution 0.4×0.4×3 mm) scans in transaxial, coronal, and sagittal orientations to acquire data covering the extent of the tumor. BOLD data were acquired as a series of 60 repeats of a volumetric echo-planar T2-weighted sequence (TR: 3000 ms, TE: 35 ms, resolution 1.6×1.6×4 mm) collected over a period of 3 minutes. In addition, a single slice fast field echo (FIFE) sequence (resolution 0.5×0.5×4 mm) was used to acquire 8 images at echo times from 5 to 75 ms, allowing fitting of pixel T2* values. After these acquisitions, papaverine was delivered intravenously and the BOLD and FEE data were acquired again one hour post-administration. All data were analyzed using the RT Image software package. BOLD data were registered using an affine transformation and percent signal change was calculated on a per-pixel basis: T2* values were fit on a pixel-by-pixel basis using a Levenberg-Marquardt least squares fit to a monoexponential decay function. A three-dimensional region-of-interest spanning the tumor was defined on the basis of the T2-weighted images and was used to calculate the median change in BOLD signal intensity and T2* over the tumor, as well as the fraction of the tumor that exhibited BOLD signal changes greater than 1, 2, 5, and 10% and that exhibited T2* increases of 1, 2, 5, and 10 MS.

Statistics. Data are presented as mean values standard deviation. Student's t-test was used for calculation of significance in differences for pairwise comparisons. In the figures shown, a significance level of p≤0.05 is marked with *, p≤0.01 with **, and p≤0.001 with ***.

Results

3.1. PODD-Luc signal increases under hypoxia in vitro and in vivo. The pODD-Luc reporter system has been previously used to monitor levels of hypoxia in vitro and in vivo, as

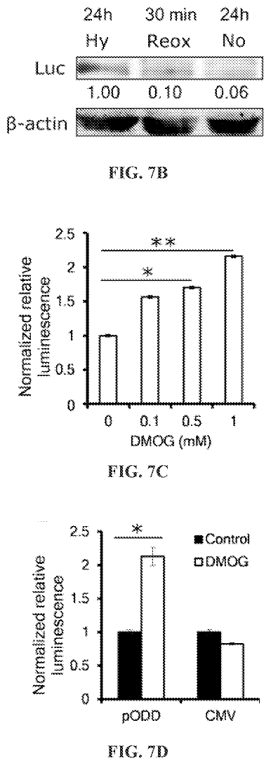

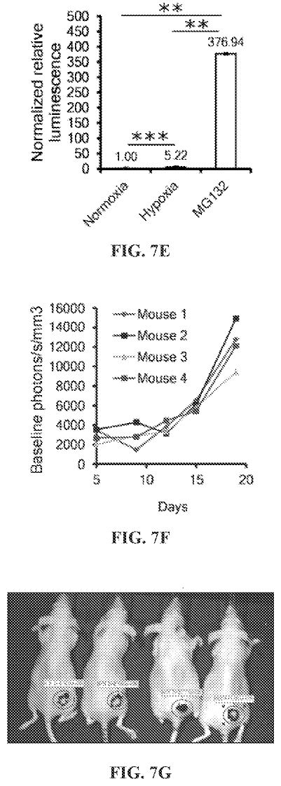

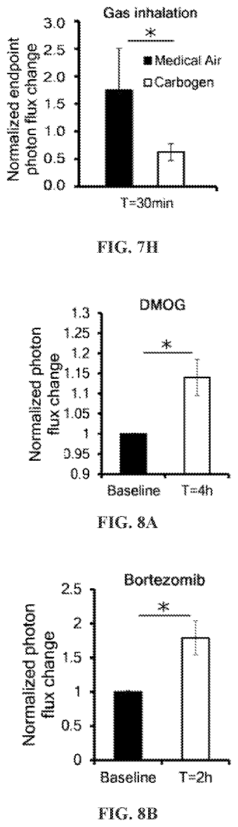

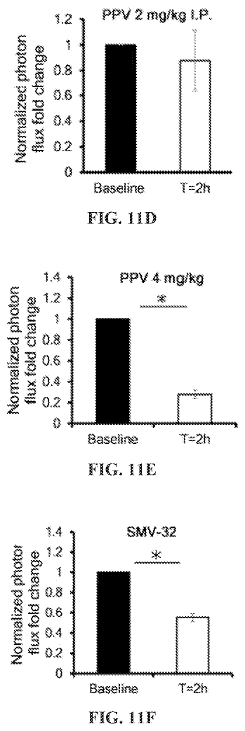

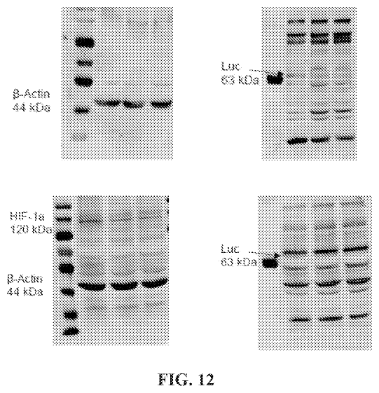

was first reported by the Kaeiin group [24]. This reporter is a fusion protein connecting the oxygen dependent degradation domain (ODD) of the HIF1α protein fused to the firefly luciferase reporter. The ODD confers oxygen lability on the luciferase enzyme which can be stabilized in hypoxia, similar to the HIF1α protein. We have subcloned the original chimeric gene into a bicistronic vector with puromycin resistance cassette to generate pODD-Luc-Puro. This transgene was introduced into HeLa cervical (HeLa pODD) and MiaPaca-2 pancreatic ire (MP2 pODD) cancer cell lines and puromycin resistant pools selected. We first confirmed the function of the reporter in vitro. We found that the activity of the pODD-Luc reporter cells increased luciferase activity after 24 h in 1% oxygen. We observed more than 9-fold increase of luminescence and 10-fold increase in reporter protein when compared to either 21% oxygen conditions or after 10 minutes of reoxygenation from hypoxic treatment (FIG. 7A-B, FIGS. 11A-11B). Likewise, we tested luciferase response in cells treated with dimethyloxalyglycine (DMOG) that inhibits the prolyl hydroxylases (PHDs) that target HIF1α for degradation. We found that 16 hours of increasing concentrations of DMOG increased pODD-Luc signal in a dose dependent manner ranging up to 2.2× at 1 mM (FIG. 7C).

We confirmed that the increase in pODD-Luc signal by DMOG was because of reporter stabilization due to PHD inhibition by generating control MiaPaca-2 cells expressing constitutive luciferase reporter gene expression under CMV promoter (MP2 CMV), which were unresponsive to DMOG treatment (FIG. 7D). Because HIF1α is destroyed by proteasomal degradation, we next treated MP2 pODD cells with proteasome inhibitor MG132 and found a dramatic 70-fold increase in pODD-Luc activity when compared to 24 h hypoxia and 377-fold induction when compared to normoxia suggesting that hypoxia stabilizes only a fraction of the expressed reporter protein in vitro (FIG. 7E). Finally, to evaluate pODD-Luc reporter activity in vivo, we implanted HeLa pODD into the flanks of immunosuppressed athymic nu/nu mice. We found that the luciferase signal increased as the tumors grew, however, when we normalized for tumor volume, we found that the signal per mm³ of tumor also increased, suggesting that as the tumor grew, the amount of hypoxia with it also increased (FIG. 7F-G). To confirm that the luciferase signal was responsive to tumor oxygenation, we measured the luciferase signal in MP2 ODD tumor bearing animals after 30 minutes of breathing either medical air, or carbogen (95% 02, 5% CO₂). As expected, the animals breathing carbogen had approximately ⅓ the luciferase signal of the animals breathing medical air (FIG. 714).

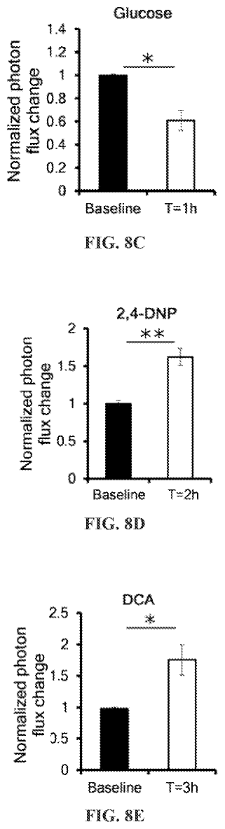

3.2. OCR manipulation modulates levels of luclerase activity in vivo. Based on our previous findings, our model predicts that biochemical modification of oxygen consumption will affect the overall oxygenation of the tumor [19]. We therefore grew orthotopic pancreatic and heterotopic flank MP2 pODD tumors in immunosuppressed nu/nu mice and monitored relative levels of luciferase activity in real time using IVIS imaging system. To further confirm that the luciferase signal is a reflection of HIFla protein stability in vivo, we evaluated the its impact of inhibiting either the PHDs using DMOG or the proteasome using bortezomib on tumor luciferase activity. As we reported for these agents in vitro, in heterotopic MP2 pODD tumor-bearing mice we found that treatment with either 4 mg dose of DMOG or 1 mg/kg bortezomib delivered intraperitoneally resulted in a significant increase of photon flux at 4 and 2 hours post injection, respectively (FIGS. 8A-B).

Also, as we had seen in vitro, the effect of proteasomal inhibition caused a greater increase in signal than P1-ID inhibition. The control treatment with vehicle showed no effect on photon flux (FIG. 11C). Next, we evaluated the effect of manipulating mitochondrial oxidative metabolism on the dynamic changes of tumor hypoxia. One way to suppress mitochondrial function is to provide cells with a bolus of glucose to produce the Crabtree effect [17] where metabolism shifts from oxidative to glycolytic. Indeed, in mice harboring heterotopic MP2 pODD flank tumors, a 2 glikg intraperitoneal glucose injection (following an overnight fast) showed a 40% reduction of photon flux at 1 hour (FIG. 8C). We then investigated whether OCR stimulating drugs would in turn cause an elevation of hypoxic luciferase activity. Mitochondrial uncouplers such as 2,4-dinitrophenol (2,4-DNP) are protonophoric compounds that dissipate the mitochondrial proton gradient and stimulate a rapid increase of OCR. A single intraperitoneal dose of 20 mg/kg 2,4-DNP produced a significant 1.6× increase of photon flux compared to baseline (FIG. 8D). Finally Dichloroacetate (DCA) is an inhibitor of pyruvate dehydrogenase kinases1-4 (PDHKs). Under hypoxic conditions, PDHKs add inhibitory phosphorylations to pyruvate dehydrogenase PDHAI subunit to prevent pyruvate oxidation in the TCA cycle. Inhibition of PDHKs by DCA has been shown to temporarily increase mitochondrial OCR [8,25]. Similar to 2,4-DNP, intraperitoneal treatment with 75 mg/kg DCA caused a 1.8× increase of photon flux, further supporting the model that pharmacological alteration in OMCR impacts tumor oxygenation (FIG. 8E).

Alitochoncfrial complex 1 inhibitors papaverine and SMV-32 effectively reduce hypoxia in orthotopic pancreas cancers. We have previously shown that papaverine (PPV), an FDA-approved vasorelaxant effectively reduce hypoxia in model breast and lung tumors by a previously unrecognized activity inhibiting mitochondrial complex I[19].

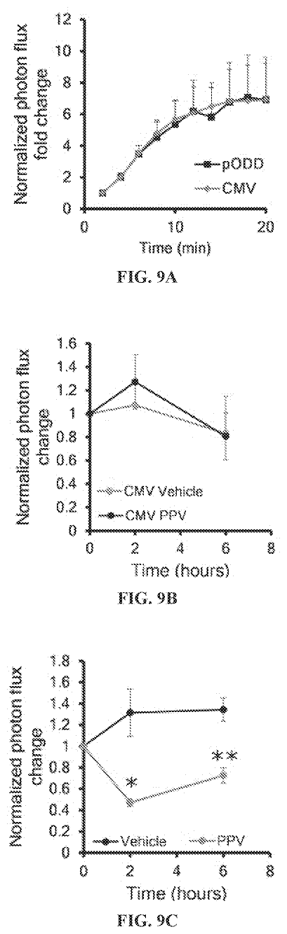

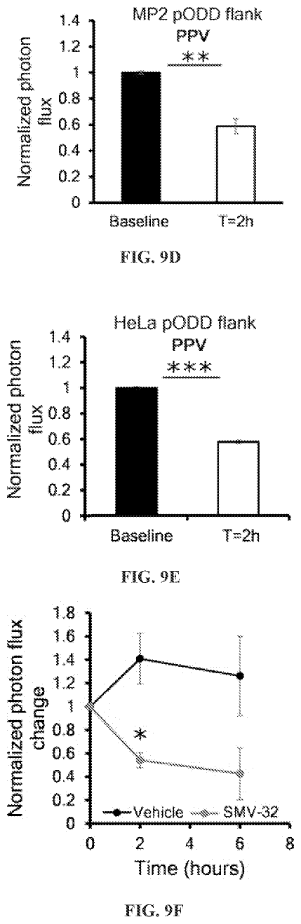

Furthermore, we have designed novel derivative of PPV, SMV-32, that has reduced the vasoactive PDE 10A inhibitor activity and enhanced pharmacokinetic parameters. To evaluate the pharmacodynamics properties of PPV and SMV-32 in pancreatic tumors, we grew both orthotopic MP2 pODD and MP2 CMV tumors in immunocompromised nu/nu mice. Both tumor models showed comparable baseline reporter signal in response to D-luciferin (FIG. 9A), To rule out non-specific activity of PPV to increase tumor luciferase activity, we tested the oxygen-independent Ni1P2 CMV tumor-beating mice with a single dose of 2 mg/kg PPV or vehicle by tail vein injection and found no difference in CMV luciferase activity up to 6 hours post injection (FIG. 9B). When we treated MP2 pODD mice with PPV we observed 45% reduction in photon flux compared to vehicle (FIG. 9C). A similar response was observed in nulnu mice with flank MP2 pODD and HeLa pODD tumors treated with PPV or SMV-32 (FIG. 9D-E, FIG. 11F). We also determined dose and route of delivery effects of PPV on ODI)-1uc activity. Intraperitoneal delivery of 2 mg/kg PPV did not reduce the photon flux suggesting reduced pharmacokinetics properties of intraperitoneal administration. However, a 4 mg/kg dose by tail vein showed greater photon flux reduction than the 2 mg/kg dose, suggesting a dose-dependent response (FIGS. 11 D-11E). When we compared 2 mg/kg of each SMV-32 to PPV in orthotopic pODD tumors, we found that the photon flux of SMV-32 treated mice trended to longer duration of action and lower luciferase activity at 6 hours when compared to PPV treated mice (FIG. 9C and 9F).

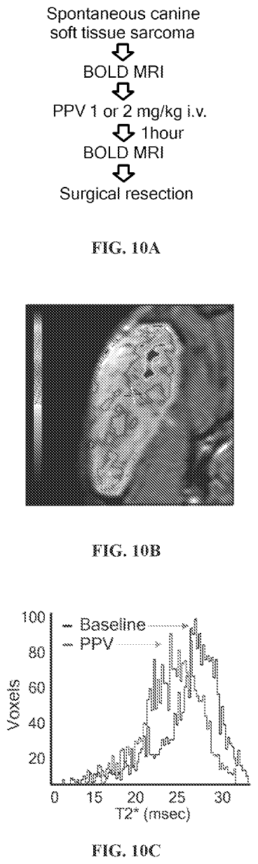

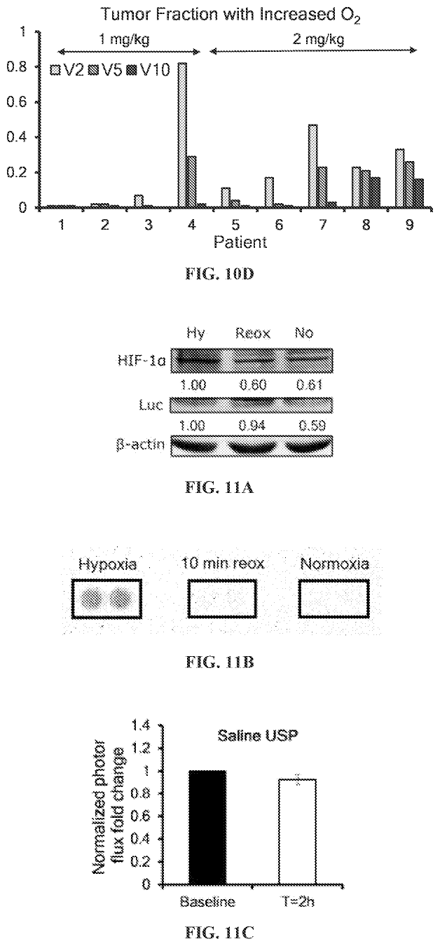

3.4. Mitochondrial complex 1 inhibitor papaverine increases oxygenation in spontaneous canine sarcomas. The murine models of transplanted tumors are genetically simple for both host and tumor genomes. To better model complex heterogeneous human cancers, we have begun an NCI funded animal study in client dogs being treated for cancer at the OSU Veterinary Medical Center integrative cancer clinic. This pharmacodynamics study uses blood oxygen level determination (BOLD) NMI to determine tumor oxygenation at baseline and one hour after a clinically acceptable dose of 1 or 2 mg/kg PPV delivered intravenously (FIG. 10A). When we overlay these images, we can detect the differences in BOLD T2* relaxation signal changes in a voxel-by-voxel analysis. The image in FIG. 10B is pseudocolored to show changes from zero (transparent) to 10 ms (red) increase in T2* signal. The pattern of changes is reminiscent of the heterogenous pattern of hypoxia that has been reported to exist within a tumor. Histogram analysis of the patient in 4B revealed a clear separation between pre- and post-treatment T2* values (FIG. 10C). Panel 4D is a summary of the first 9 evaluatable patient images (four treated with 1 mg/kg and five with 2 mg/kg). Because of the heterogeneity seen in these images, we decided to report fractional tumor volumes that increased T2* by either 2 ms, 5 ms or 10 ms. This level of change is consistent with what has been reported for T2* increases in tumor bearing mice before and after carbogen breathing, with median changes of only 1-2 ins, but heterogeneous regions of the tumor increasing up to 10 ms [26].

This relative change is consistent with the murine ODD-iuc data presented above showing a change in signal that is relatively similar between breathing carbogen (FIG. 7) and after delivery of 2 mg/kg PPV (FIG. 9). Within this heterogeneous response, we find a greater fraction of tumors responding well to the higher dose of 2 mg/kg (3/5) than those responding well to 1 mg/kg (1/4). Signal noise is approximately 1-2 ms, so signal theory would indicate that signals >2 noise would indicate non-random increases in T2*.

4. Discussion

Non-invasive analysis of biologically significant hypoxia with ODDluciferase offers a powerful technology to investigate oxygenation in model tumors. In this study, we show that ODDluciferase signal is responsive to chemical modification of the HIF1α degradation pathway in vitro and in vivo. These results give confidence to use it in the analysis of strategies that modify mitochondrial function to change hypoxia. Agents that inhibit mitochondrial function decrease hypoxia and luciferase signals, while agents that stimulate mitochondria increase hypoxia and luciferase signal. This paradigm appears to hold for both homogeneous murine models and heterogenous spontaneous canine tumors where hypoxia has been reported [27,28].

One limitation to these imaging modalities to measure hypoxia is the heterogeneity existing on the cellular level. While it is true that as tumors get larger they show increased hypoxia in the core [29] (FIG. 7F), but this is an average of many smaller volumes that have a range of oxygenations. The use of hypoxia marker drugs such as pimonidazole or EFS [31] have shown that hypoxia can be spread through the tumor in patterns indicative of both diffusion and perfusion limited oxygen delivery. These regions can be on the level of tens or hundreds of cells, far too small to detect from an imaging technology, but potentially important clinically as these resistant cells can participate in tumor regrowth after treatment [32].

Tumor hypoxia has remained a barrier to effective anti-cancer therapy for decades after it was recognized as causing radiation resistance [33]. No clinically strategy has been approved by the FDA to reduce hypoxia in order to augment standard therapies. Many attempts have been made to increase oxygen delivery to tumors without clinical success [34]. These failures are partly due to limitations in tumor vessel function. Tumor vessels typically are tortuous, contain breaks and blind ends, and lack a muscular wall. For this reason, even if more oxygen is delivered systemically, the amount that gets to the tumor core is limited. Our alternative approach has been to reduce oxygen demand within the tumor cells. Inhibitors of mitochondrial function appear to be a promising strategy to increase oxygenation in model murine tumors and spontaneous canine soft tissue sarcoma.

Our findings support the model that drugs that modify mitochondrial metabolism can regulate tumor hypoxia, potentially generating therapeutic gain,

### REFERENCES

1. Thomlinson, R. H.; Gray, L. H. The histological structure of some human lung cancers and the possible implications for radiotherapy. British journal of cancer 1955, 9, 539-549.
2. Kimura, H.; Braun, R. D.; Ong, E. T.; Hsu, R.; Secomb, T. W.; Papahadjopoulos, D.; Hong, Dewhirst, M. W. Fluctuations in red cell flux in tumor microvessels can lead to transient hypoxia and reoxygenation in tumor parenchyma. Cancer research 1996, 56, 5522-5528.
3. Bader, S. B.; Dewhirst, M. W.; Hammond, E. M. Cyclic Hypoxia: An Update on Its Characteristics, Methods to Measure It and Biological Implications in Cancer, Cancers 2021, 13, 23.
4, Wykoff, C. C.; Beasley, N. J.; Watson, P. H.; Turner, K. J.; Pastorek, J.; Sibtain, A.; Wilson, G. D.; Turley, H.; Talks, K. L.; Maxwell, P. H.; et al. Hypoxia-inducible expression of tumor-associated carbonic anhydrases. Cancer research 2000, 60, 7075-7083.
5. Wang, G. L.; Semenza, G. L. General involvement of hypoxia-inducible factor 1 in transcriptional response to hypoxia. Proc. Natl. Acad. Sci. U. S. A. 1993, 90, 4304-4308, doi:10.1073/pnas.90.9.4304.

6. Aprelikova, O.; Chandramouli, G. V.; Wood, M.; Vasselli, J. R.; Diss, J.; Maranchie, J. K.; Linehan, W. M.; Barrett, J. C. Regulation of HIF prolyl hydroxylases by hypoxia-inducible factors. Journal of cellular biochemistry 2004, 92, 491-501, doi:10.11.002/jcb. 20067.
7. Wang, G. L,; Semenza, G. L. Characterization of hypoxia-inducible factor 1 and regulation of DNA binding activity by hypoxia. The Journal of Biological Chemistry 1993, 268. 21513-21518.
8. Papandreou, I.; Cairns, R. A.; Fontana, L. Lim, A. L.; Denko, N. C. HIF-1 mediates adaptation to hypoxia by actively downregulating mitochondrial oxygen consumption. Cell metabolism 2006. 3, 187-197, doi:10.1016/j.cmet.2006.01.012.
9. Kim, J. W.; Tchernyshyov, I.; Semenza, G. L.; Dang, C. V. HIF-1-mediated expression of pyruvate dehydrogenase kinase: a metabolic switch required for cellular adaptation to hypoxia. Cell metabolism 2006, 3, 177-185, doi:10.1016/j.cmet.2006.02.002.
10. Vaupel, P. Hypoxia and aggressive tumor phenotype: implications for therapy and prognosis. Oncologist 2008, 13 Suppl 3, 21-26, doi:10.1634/theoncologist.13-S3-21.
11. Gomez, C. R. Editorial: Tumor Hypoxia: Impact in Tumorigenesis, Diagnosis, Prognosis, and Therapeutics. Frontiers in oncology 2016, 6, doi:10.3389/fonc.2016.00229.
12. Overgaard, J, Hypoxic radiosensitization: adored and ignored. Journal of clinical oncology: official journal of the American Society of Clinical Oncology 2007, 25, 4066-4074, doi:10.1200/jco.2007.12.7878.
13. Sorensen, B. S.; Horsman, M. R. , Tumor Hypoxia: Impact on Radiation Therapy d Molecular Pathways. Frontiers in oncology 2020, 10, doi:10.3389/fonc.2020.00562.
14. Snyder, C. M. , Chandel, N. S. Mitochondrial regulation of cell survival and death during low-oxygen conditions. Antioxidant & redox signaling 2009, 11, 2673-2683, doi:10.1089/ars.2009.2730.
15. Mitchell, P. Coupling of Phosphorylation to Electron and Hydrogen Transfer by a Chemi-Osmotic type of Mechanism. Nature 1961, 19/, 144-148, doi:10.1038/191144a0.
16. Zannella, V. E.; Dal Pra, A.; Muaddi, H.; MCKee, T. D.; Stapleton, S.; Sykes, J.; Glicksman, R.; Chaib, S.; Za. miara, P.; Milosevic, M.; et al. Reprogramming metabolism with metformin improves tumor oxygenation and radiotherapy response. Clinical cancer research: an official journal of the American Association for Cancer Research 2013, 19, 6741-6750, doi:10.1158/1078-0432.ccr-13-1787.
17. Snyder, S. A.; Lanzen, J. L.; Braun, R. D.; Rosner, G.; Secomb, T. W. , Biaglow, J.; Brizel :1/1\1; Dewhirst, M. W. Simultaneous administration of glucose and hyperoxic gas achieves greater improvement in tumor oxygenation than hyperoxic gas alone. International journal of radiation oncology, biology, physics 2001, 51, 494-506.
18. Molina, J. R.; Sun, Y.; Protopopova, M.; Gera, S.; Bandi, M.; Bristow, C.; McAfoos, T.; Morlacchi, P.; Ackroyd, J.; Agip, A. A,, et al. An inhibitor of oxidative phosphorylation exploits cancer vulnerability. Nature medicine 2018, 24, 1036-1046, doi:10.1038/s41591-018-0052-4.
19. Benej, M.; Hong, X.; Vibhute, S.; Scott, S.; Wu, J.; Graves, E.; Le, Q. T.; Koong, A. C.; Giaccia, A. J. , Yu, B.; et al. Papaverine and its derivatives radiosensitize solid tumors by inhibiting mitochondrial metabolism. Proc Nati Acad SCi U Sal 2018, 115, 10756-10761, doi:10.1073/pnas.1808945115.
20. Ashton, T. M.; Fokas, F.; Kunz-Schughart, L. A.; Folkes, L. K. , Anbalagan, S.; Huether, M.; Kelly, C. J.; Pirovano, G.; Buffa, F. M.; Hammond, E. M. , et al. The anti-malarial atovaquone increases radiosensitivity by alleviating tumour hypoxia. Nature communications 2016, 7, 12308, doi:10.1038/ncomms12308.
21. O'Connor, J. P. B.; Robinson, S. P.; Waterton, J. C. Imaging tumour hypoxia with oxygen-enhanced -Mill and BOLD MRI. The British journal of radiology 2019, 92, 20180642, doi:10.1259/bjr.20180642.
22. Padhani, A. R.; Krohn, K. A.; Lewis, J. S., Alber, M. Imaging oxygenation of human tumours. European radiology 2007, 17, 861-872. doi:10.1007/s00330-006-0431-y.
23. Euhus, D. M.; Hudd, C.; LaRegina, M. C.; Johnson, F. E. Tumor measurement in the nude mouse. Journal of surgical oncology 1986, 31, 229-234, doi:10.1002/jso.2930310402.
24. Safran, M.; Kim, W.Y.; O'Connell, F.; Flippin, L.; Giinzler, V.; Horner, J. W.; Depinho, R. 4.; Kaelin, W. G., Jr. Mouse model for noninvasive imaging of HIF prolyl hydroxylase activity: assessment of an oral agent that stimulates erythropoietin production. Proc Natl Acad Sci U S A 2006, 103, 105-110, doi:10.1073/pnas. 0509459103.
25. Papandreou, I.; Goliasova, T.; Denko, N,C. Anticancer drugs that target metabolism: Is dichloroacetate the new paradigm? International journal of cancer 2011, 128, 1001-1008, doi:10.1002/ijc.25728,
26. Baudelet, C.; Gallez, B. How does blood oxygen level-dependent (BOLD) contrast correlate with oxygen partial pressure (pO2) inside tumors? Magnetic resonance in medicine 2002, 48, 980-986, doi:10.1002/mrm.10318,
27. Bruehlmeier, M.; Kaser-Hotz, B.; Achermann, R.; Bley, C. R.; Wergin, M.; Schubiger, P. A.; Ametamey, S. M. Measurement of tumor hypoxia in spontaneous canine sarcomas. Veterinary radiology & ultrasound: the official journal of the American College of Veterinary Radiology and the International Veterinary Radiology Association 2005, 46, 348-354, doi:10.1111/j.1740-8261.2005.00065.x.
28. Thrall, D. E.; Rosner, G. L,, Azuma, C.; McEntee, M. C.; Raleigh, J. A. Hypoxia marker labeling in tumor biopsies: quantification of labeling variation and criteria for biopsy sectioning. Radiotherapy and oncology journal of the European Society for Therapeutic Radiology and Oncology 1997, 44, 171-176, doi:10.1011.6/s0167-8140(97)01931-2.
29. Klibanov, A. L.; Hu, S. Monitoring Oxygenation Levels Deep in the Tumor Core: Noninvasive Imaging of Hypoxia, Now in Real-Time 3D. Cancer research 2019, 79, 4577-4579, doi:10.1158/0008-5472. can-19-2151.
30. Varia, M. A.; Calkins-Adams, D. P.; Rinker, L. H.; Kennedy, A. S.; Novotny, D. B.; Fowler, W. C. , Jr.; Raleigh, J. A. Pimonidazole: a novel hypoxia marker for complementary study of tumor hypoxia and cell proliferation in cervical carcinoma. Gynecologic oncology 1998, 71, 270-277, doi:10.1006/gyno.1998.5163.
31. Ziemer, L. S.; Evans, S. M.; Kachur, A. V.; Shuman, A. L.; Cardi. , C. A.; Jenkins, W. T.; Karp, J. S.; Alavi, A.; Dolbier, Jr.; Koch, C. J. Noninvasive imaging of tumor hypoxia in rats using the 2-nitroimidazole 18F-EF5. European journal of nuclear medicine and molecular imaging 2003, 30, 259-266, doi:10.1007/s00259-002-1037-5.
32. Brown, J. M. Evidence for acutely hypoxic cells in mouse tumours, and a possible mechanism of reoxygenation. The British journal of radiology 1979, 52, 650-656, doi:10.1259/0007-1285-52-620-650.
33. Gray, L. H.; Conger, A. D.; Ebert, M.; Hornsey, S.; Scott, O. C. The concentration of oxygen dissolved in tissues at the time of irradiation as a factor in radiotherapy. The British journal of radiology 1953, 26, 638-648, doi:10,1259/0007-1285-26-312-638,
34. Torrisi, F.; \iicario, N.; Spitale, F. M.; Cammarata, F. P.; Minafra, L.; Salvatorelli, L.; Russo, G.; Cuttone, G.; Valable, S.: Gulino, R.; et al. The Role of Hypoxia and SRC Tyrosine Kinase in Glioblastoma invasiveness and Radioresistance. Cancers 2020, 12, doi:10,3390/cancers12102860.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

1. A method for treating cancer in a subject comprising:
(a) administering to the subject a therapeutically effective amount of one or more compounds to inhibit mitochondrial oxygen consumption in a cancer cell, the one or more compounds having a structure according to Formula I 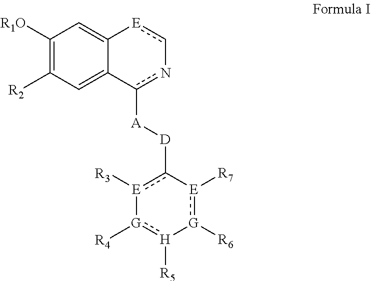
wherein
A and D can be independently present or absent and are independently selected from CR′R″, NR′, and O, wherein R′ and R″ are independently for each occurrence selected from hydrogen, hydroxyl, halogen, amine, alkylamine, C₁-C₆ alkyl, C₁-C₆ alkyl halide, C₁-C₆ alkoxy, or R′ and R″ combine together with the atom to which they are attached form a carbonyl group;
E, G, and H can be independently selected from C, N′, O, and S;
R¹ and R² can be independently selected from hydrogen, C₁-C₆ alkyl, C₁-C₆ alkyl halide, C₁-C₆ alkoxy, and C₁-C₆ alkylamine;
R³ to R⁷ can be independently selected from hydrogen, hydroxyl, C₁-C₆ alkyl, C₁-C₆ alkyl halide, C₁-C₆ alkoxy, and C₁-C₆ alkylamine or R³ and R⁴ or R⁴ and R⁵ or R⁵ and R⁶ or R⁶ and R⁷ combine together with the atoms to which they are attached form a C₅-C₈ aryl or heteroaryl, or C₅-C₈ cycloalkenyl or heterocycloalkenyl; wherein R³ to R⁷ are independently unsubstituted or substituted with hydroxyl, halogen, C₁-C₃ alkyl, C₁-C₃ alkenyl, or C₁-C₃ alkyl halide; and [symbol]
represents a bond and is independently for each occurrence absent or present; and
(b) administering an immunotherapy to treat the cancer.

2. A method for treating cancer in a subject comprising:
(a) administering to the subject a pharmaceutical composition to inhibit mitochondrial oxygen consumption in a cancer cell, the pharmaceutical composition comprising one or more compounds having a structure according to Formula I 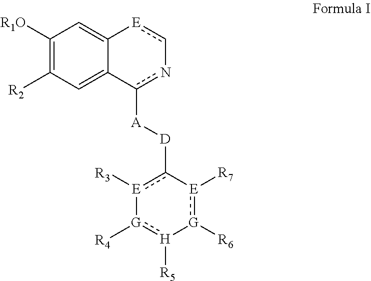
wherein
A and D can be independently present or absent and are independently selected from CR′R″, NR′, and O, wherein R′ and R″ are independently for each occurrence selected from hydrogen, hydroxyl, halogen, amine, alkylamine, C₁-C₆ alkyl, C₁-C₆ alkyl halide, C₁-C₆ alkoxy, or R′ and R″ combine together with the atom to which they are attached form a carbonyl group;
E, G, and H can be independently selected from C, N′, O, and S;
R¹ and R² can be independently selected from hydrogen, C₁-C₆ alkyl, C₁-C₆ alkyl halide, C₁-C₆ alkoxy, and C₁-C₆ alkylamine;
R³ to R⁷ can be independently selected from hydrogen, hydroxyl, C₁-C₆ alkyl, C₁-C₆ alkyl halide, C₁-C₆ alkoxy, and C₁-C₆ alkylamine or R³ and R⁴ or R⁴ and R⁵ or R⁵ and R⁶ or R⁶ and R⁷ combine together with the atoms to which they are attached form a C₅-C₈ aryl or heteroaryl, or C₅-C₈ cycloalkenyl or heterocycloalkenyl; wherein R³ to R⁷ are independently unsubstituted or substituted with hydroxyl, halogen, C₁-C₃ alkyl, C₁-C₃ alkenyl, or C₁-C₃ alkyl halide; and [symbol]
represents a bond and is independently for each occurrence absent or present; and
(b) administering an immunotherapy to treat the cancer.

3. The method of claim 1, wherein the one or more compound(s) is selected from the group consisting of papaverine, 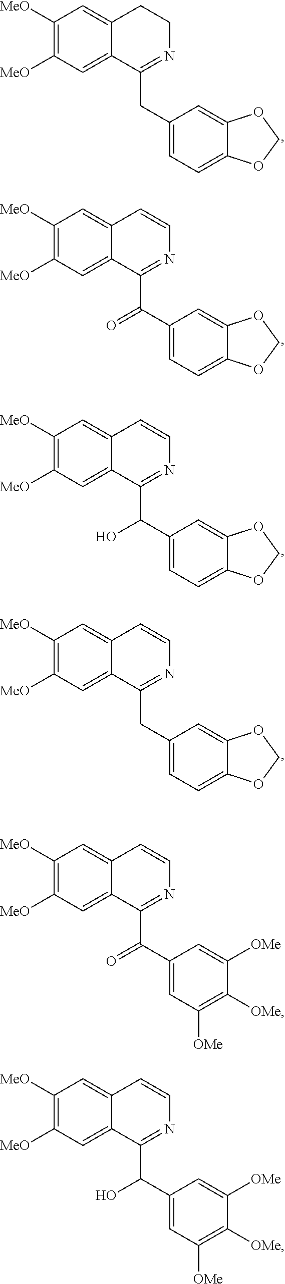 [SMILES: COc1cc2c(cc1OC)C(Cc1ccc3c(c1)OCO3)=NCC2 ; COc1cc2ccnc(C(=O)c3ccc4c(c3)OCO4)c2cc1OC ; COc1cc2ccnc(C(O)c3ccc4c(c3)OCO4)c2cc1OC ; COc1cc2ccnc(Cc3ccc4c(c3)OCO4)c2cc1OC ; COc1cc2ccnc(C(=O)c3cc(OC)c(OC)c(OC)c3)c2cc1OC ; COc1cc2ccnc(C(O)c3cc(OC)c(OC)c(OC)c3)c2cc1OC] 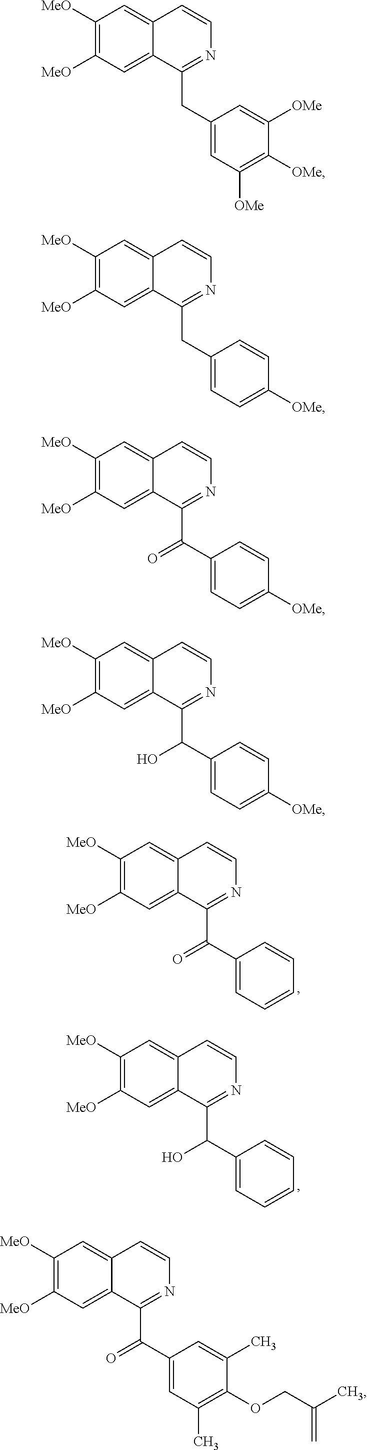 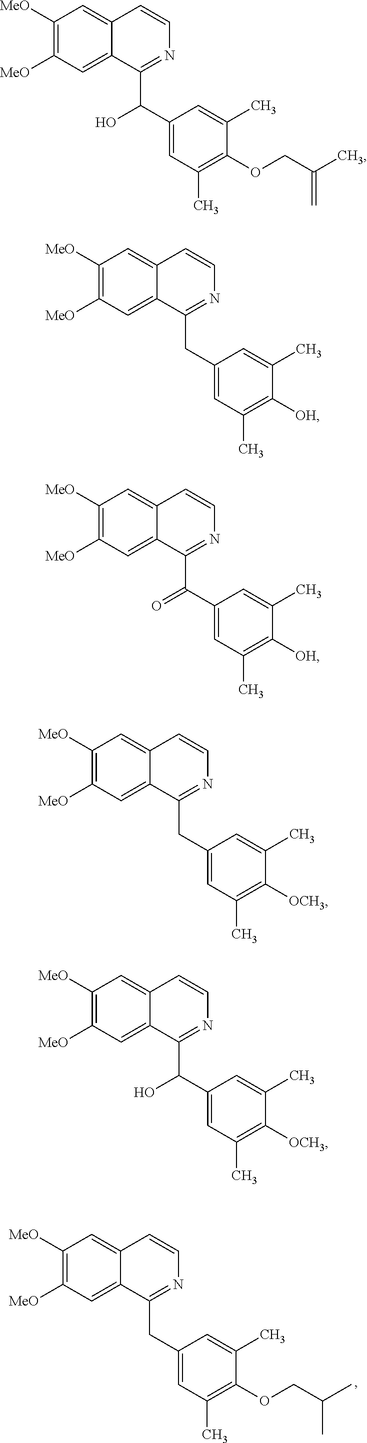 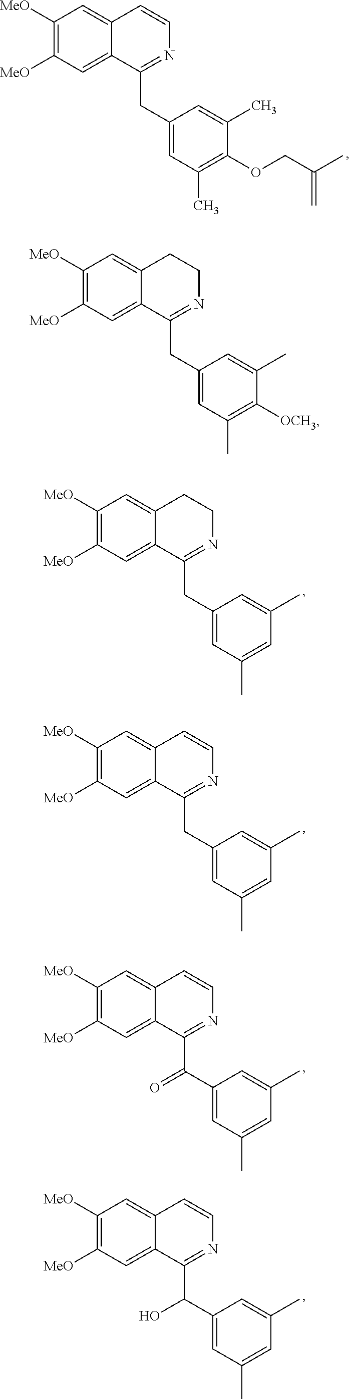 [SMILES: C=C(C)COc1c(C)cc(Cc2nccc3cc(OC)c(OC)cc23)cc1C ; COc1cc2c(cc1OC)C(Cc1cc(C)c(OC)c(C)c1)=NCC2 ; COc1cc2c(cc1OC)C(Cc1cc(C)cc(C)c1)=NCC2 ; COc1cc2ccnc(Cc3cc(C)cc(C)c3)c2cc1OC ; COc1cc2ccnc(C(=O)c3cc(C)cc(C)c3)c2cc1OC ; COc1cc2ccnc(C(O)c3cc(C)cc(C)c3)c2cc1OC] 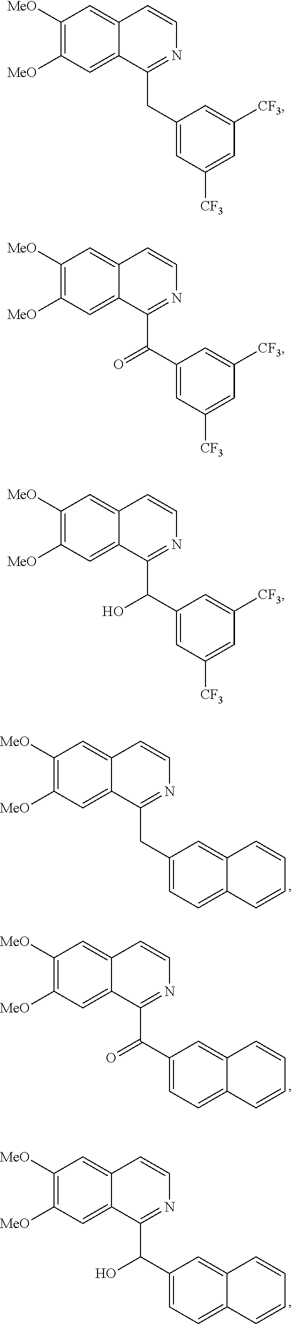 [SMILES: COc1cc2ccnc(Cc3cc(C(F)(F)F)cc(C(F)(F)F)c3)c2cc1OC ; COc1cc2ccnc(C(=O)c3cc(C(F)(F)F)cc(C(F)(F)F)c3)c2cc1OC ; COc1cc2ccnc(C(O)c3cc(C(F)(F)F)cc(C(F)(F)F)c3)c2cc1OC ; COc1cc2ccnc(Cc3ccc4ccccc4c3)c2cc1OC ; COc1cc2ccnc(C(=O)c3ccc4ccccc4c3)c2cc1OC ; COc1cc2ccnc(C(O)c3ccc4ccccc4c3)c2cc1OC] 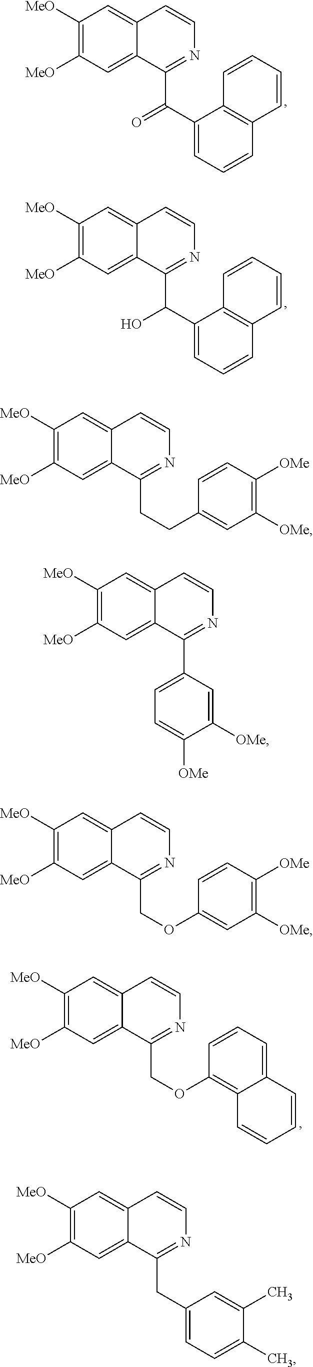 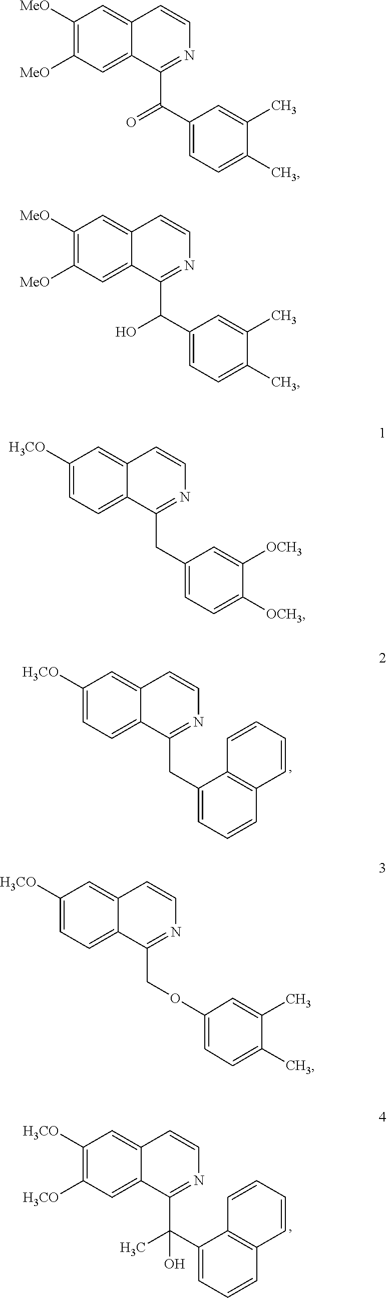 [SMILES: COc1cc2ccnc(C(=O)c3ccc(C)c(C)c3)c2cc1OC ; COc1cc2ccnc(C(O)c3ccc(C)c(C)c3)c2cc1OC ; COc1ccc2c(Cc3ccc(OC)c(OC)c3)nccc2c1 ; COc1ccc2c(Cc3cccc4ccccc34)nccc2c1 ; COc1ccc2c(COc3ccc(C)c(C)c3)nccc2c1 ; COc1cc2ccnc(C(C)(O)c3cccc4ccccc34)c2cc1OC] 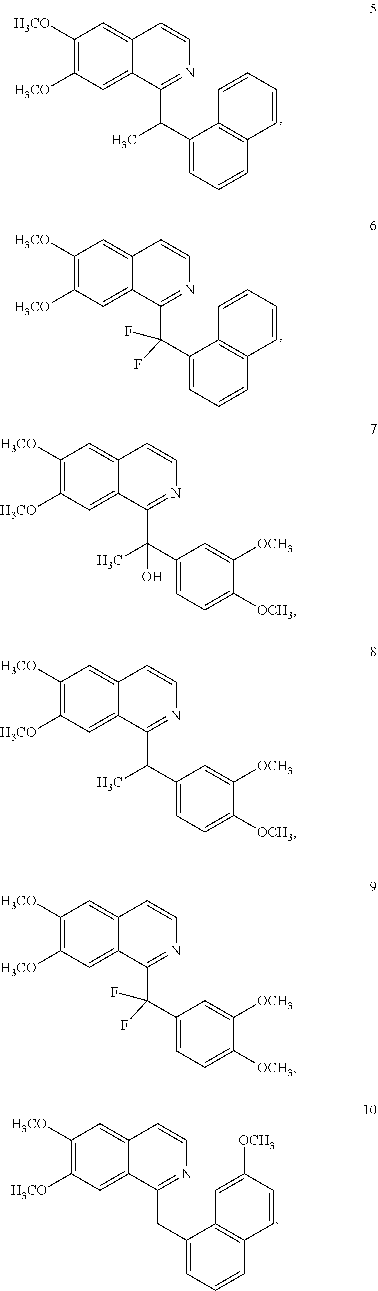 [SMILES: COc1cc2ccnc(C(C)c3cccc4ccccc34)c2cc1OC ; COc1cc2ccnc(C(F)(F)c3cccc4ccccc34)c2cc1OC ; COc1ccc(C(C)(O)c2nccc3cc(OC)c(OC)cc23)cc1OC ; COc1ccc(C(C)c2nccc3cc(OC)c(OC)cc23)cc1OC ; COc1ccc(C(F)(F)c2nccc3cc(OC)c(OC)cc23)cc1OC ; COc1ccc2cccc(Cc3nccc4cc(OC)c(OC)cc34)c2c1] 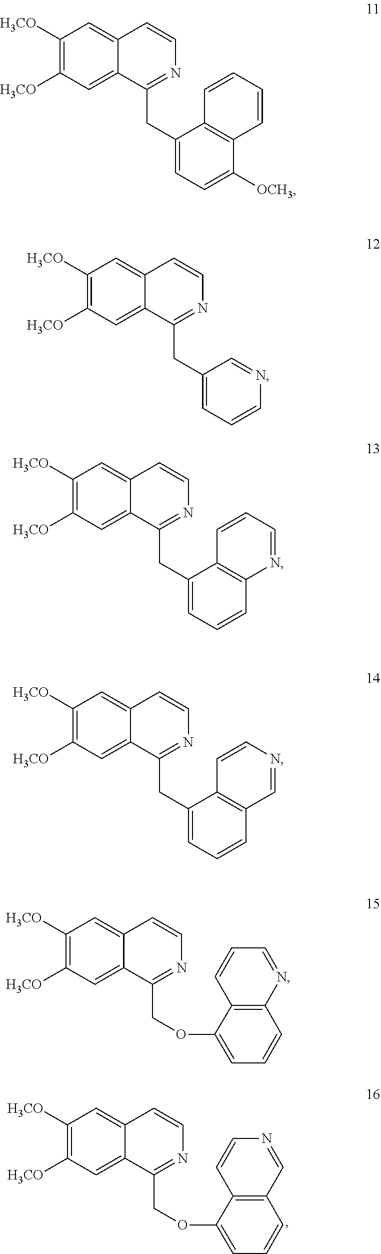 [SMILES: COc1cc2ccnc(Cc3ccc(OC)c4ccccc34)c2cc1OC ; COc1cc2ccnc(Cc3cccnc3)c2cc1OC ; COc1cc2ccnc(Cc3cccc4ncccc34)c2cc1OC ; COc1cc2ccnc(Cc3cccc4cnccc34)c2cc1OC ; COc1cc2ccnc(COc3cccc4ncccc34)c2cc1OC ; COc1cc2ccnc(COc3cccc4cnccc34)c2cc1OC] 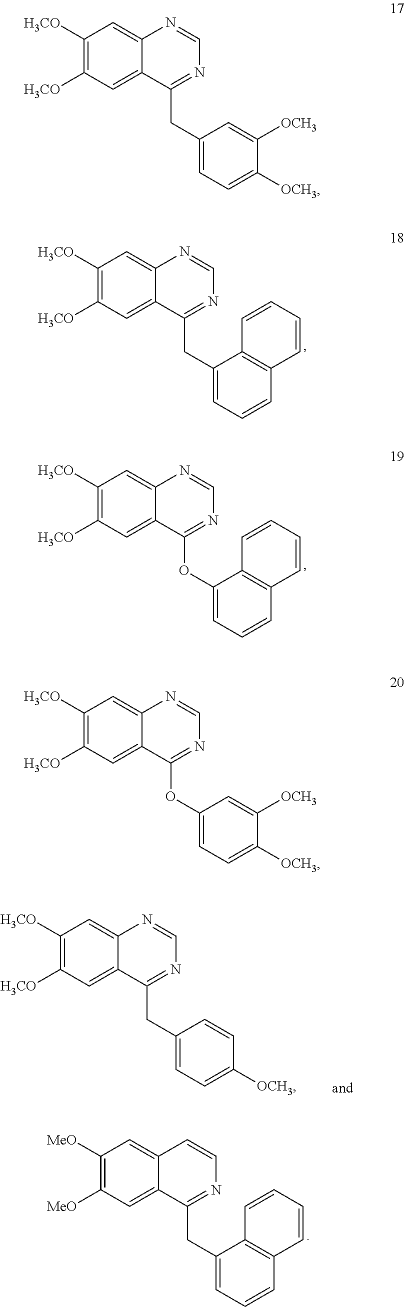 [SMILES: COc1ccc(Cc2ncnc3cc(OC)c(OC)cc23)cc1OC ; COc1cc2ncnc(Cc3cccc4ccccc34)c2cc1OC ; COc1cc2ncnc(Oc3cccc4ccccc34)c2cc1OC ; COc1ccc(Oc2ncnc3cc(OC)c(OC)cc23)cc1OC ; COc1ccc(Cc2ncnc3cc(OC)c(OC)cc23)cc1 ; COc1cc2ccnc(Cc3cccc4ccccc34)c2cc1OC]
or any combination thereof.

4. The method of claim 1, wherein the one or more compound(s) comprise:

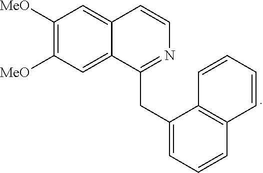 [SMILES: COc1cc2ccnc(Cc3cccc4ccccc34)c2cc1OC]

5. The method of claim 1, wherein the one or more compound(s) comprise:

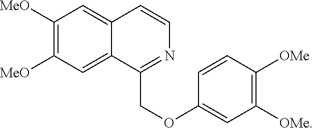 [SMILES: COc1ccc(OCc2nccc3cc(OC)c(OC)cc23)cc1OC]

6. The method of claim 1, wherein the cancer comprises a solid tumor.

7. The method of claim 1, wherein the cancer is selected from the group consisting of colorectal cancer, breast cancer, bladder cancer, gastrointestinal cancer, brain cancer, cervical cancer, head and neck cancer, lung cancer, prostate cancer, skin cancer, esophageal cancer, thyroid cancer, adrenal gland cancer, bone cancer, testicular cancer, and melanoma.

8. The method of claim 1, wherein the cancer comprises a hematological cancer.

9. The method of claim 1, wherein the cancer is selected from leukemia, myeloma, sarcoma and lymphoma.

10. The method of claim 1, wherein the immunotherapy comprises an anti-cancer immunotherapy.

11. The method of claim 1, wherein the immunotherapy comprises one or more immune checkpoint inhibitors.

12. The method of claim 1, wherein the one or more immune checkpoint inhibitors comprises a CTLA-4 inhibitor, a PD-1 inhibitor, a PD-L1 inhibitor, or any combination thereof.

13. The method of claim 1, wherein the one or more compounds, or the pharmaceutical composition is administered prior to, during, or after administering the immunotherapy.

14. The method of claim 1, wherein the one or more compounds, or the pharmaceutical composition is administered 2 days or less, 24 hours or less, 20 hours or less, 18 hours or less, 16 hours or less, 12 hours or less, 10 hours or less, 8 hours or less, 6 hours or less, 4 hours or less, 2 hours or less, 1 hour or less, or 30 minutes or less prior to administering the immunotherapy.

15. The method of claim 1, wherein the one or more compounds, or the pharmaceutical composition is administered within 5 minutes to 2 days, prior to administering the immunotherapy.

16. The method of claim 1, wherein the one or more compounds, or the pharmaceutical composition is administered at least 5 minutes, least 30 minutes, at least 2 hours, at least 6 hours, at least 14 hours, at least 1 day, at least 5 days, at least 1 week, at least 2 weeks, at least 1 month, or at least 5 months after administering the immunotherapy.

17. The method of claim 1, wherein the one or more compounds, or the pharmaceutical composition is administered within 5 minutes to 6 months.

18. The method of claim 1, wherein the method further comprises administering a chemotherapeutic drug.

19. The method of claim 1, wherein the one or more compounds or the pharmaceutical composition is administered by one or more routes selected from the group consisting of buccal, sublingual, intravenous, subcutaneous, intradermal, transdermal, intraperitoneal, oral, eye drops, parenteral, and topical administration.
